(19) United States
(12) Patent Application Publication
(10) Pub. No.: US 2022/0194945 A1
(43) Pub. Date: Jun. 23, 2022

(54) Imidazolopyridine Compounds For IRE1 Inhibition

(71) Applicant: Optikira, LLC, Cleveland, OH (US)

(72) Inventors: Richard Keenan, Cleveland, OH (US); Jon Sutton, Cleveland, OH (US); George Hynd, Cleveland, OH (US)

(21) Appl. No.: 17/433,273

(22) PCT Filed: Feb. 27, 2020
(86) PCT No.: PCT/US20/20157

§ 371 Date: Aug. 24, 2021

Related U.S. Application Data
(60) Provisional application No. 62/811,243, filed on Feb. 27, 2019.
(60) Provisional application No. 62/813,966, filed on Mar. 5, 2019.

(51) Int. Cl. C07D 487/04

(52) U.S. Cl. CPC C07D 487/04

(57) ABSTRACT

The present invention provides novel imidazolopyrazine compounds, compositions and methods for treating or preventing an IRE1α-related disease or disorder. In certain embodiments, the disease or disorder is selected from the group consisting of a neurodegenerative disease, a demyelinating disease, cancer, an eye disease, a fibrotic disease, and diabetes.

### CROSS-REFERENCE TO RELATED APPLICATIONS

The present application claims priority under 35 U.S.C. § 119(e) to U.S. Provisional Application No. 62/811,243, filed Feb. 27, 2019 and U.S. Provisional Patent Appl. No. 62/813,966, filed Mar. 5, 2019, all of which applications are incorporated herein by reference in their entireties.

### BACKGROUND OF THE INVENTION

Cells often experience conditions during which the workload on the endoplasmic reticulum (“ER”) protein folding machinery exceeds its capability, causing ER stress. ER stress can result from secretory work overload, expression of folding-defective secretory proteins, deprivation of nutrients or oxygen, changes in luminal calcium concentration, and deviation from resting redox state. Under ER stress, secretory proteins accumulate in unfolded forms within the organelle to trigger a set of intracellular signaling pathways called the Unfolded Protein Response (UPR). UPR signaling increases transcription of genes encoding chaperones, oxidoreductases, lipid-biosynthetic enzymes, and ER-associated degradation (ERAD) components.

In some instances, the ER stressed state remains too great, and cannot be remedied through the UPR's homeostatic outputs. In these situations, the UPR switches strategies and actively triggers apoptosis. Apoptosis of irremediably stressed cells is a quality control strategy that protects multicellular organisms from exposure to immature and damaged secretory proteins. Many deadly human diseases occur if too many cells die through this process. Conversely, many human diseases such as diabetes mellitus and retinopathies proceed from unchecked cell degeneration under ER stress.

IRE1α and IRE1β are ER-transmembrane proteins that become activated when unfolded proteins accumulate within the organelle. IRE1α is the more widely expressed family member. The bifunctional kinase/endoribonuclease IRE1α controls entry into the terminal UPR. IRE1α senses unfolded proteins through an ER luminal domain that becomes oligomerized during stress.

Under irremediable ER stress, positive feedback signals emanate from the UPR and become integrated and amplified at key nodes to trigger apoptosis. IRE1α is a key initiator of these pro-apoptotic signals. IRE1α employs auto-phosphorylation as a timer. Remediable ER stress causes low-level, transient auto-phosphorylation that confines RNase activity to XBP1 mRNA splicing. However, sustained kinase autophosphorylation causes IRE1α's RNase to acquire relaxed specificity, causing it to endonucleolytically degrade thousands of ER-localized mRNAs in close proximity to IRE1α. These mRNAs encode secretory proteins being co-translationally translocated (e.g., insulin in (3 cells). As mRNA degradation continues, transcripts encoding ER-resident enzymes also become depleted, thus destabilizing the entire ER protein-folding machinery. Once IRE1α's RNase becomes hyperactive, adaptive signaling through XBP1 splicing becomes eclipsed by ER mRNA destruction, which pushes cells into apoptosis.

A terminal UPR signature tightly controlled by IRE1α's hyperactive RNase activity causes (1) widespread mRNA degradation at the ER membrane that leads to mitochondrial apoptosis, (2) induction of the pro-oxidant thioredoxin-interacting protein (TXNIP), which activates the NLRP3 inflammasome to produce maturation and secretion of interleukin-1β, and consequent sterile inflammation in pancreatic islets leading to diabetes, and (3) degradation of pre-miRNA 17, leading to translational upregulation and cleavage of pre-mitochondrial caspase 2 and stabilization of the mRNA encoding TXNIP.

There is a need in the art for novel small molecule compounds that are capable of treating ER stress without resorting to UPR based apoptosis, thereby treating a wide range of disorders and diseases tied to ER stress. Such diseases include, for example, neurodegenerative diseases, demyelinating diseases, cancers, eye diseases, fibrotic diseases, and/or diabetes. The present invention meets these needs.

### BRIEF SUMMARY OF THE INVENTION

The present invention provides in one aspect compounds of formula (I):

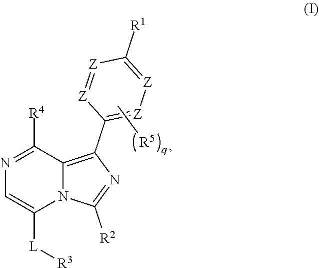

or a salt, solvate, enantiomer, diastereoisomer, isotopologue, or tautomer thereof, wherein the variables R¹-R⁵, L, Z, and q are defined elsewhere herein.

The present invention further provides methods of treating, ameliorating, or preventing diseases or disorders associated with ER stress, such as those selected from the group consisting of a neurodegenerative disease, demyelinating disease, cancer, eye disease, fibrotic disease, and diabetes. In certain embodiments, the disease or disorder is a neurodegenerative disease. In other embodiments, the disease or disorder is a demyelinating disease. In yet other embodiments, the disease or disorder is cancer. In yet other embodiments, the disease or disorder is eye disease. In yet other embodiments, the disease or disorder is a fibrotic disease. In yet other embodiments, the disease or disorder is diabetes.

### DETAILED DESCRIPTION OF THE INVENTION

The present invention relates in part to the unexpected discovery that novel inhibitors of IRE1α prevent oligomerization and/or allosterically inhibit its RNase activity.

### Definitions

As used herein, each of the following terms has the meaning associated with it in this section. Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “cancer” is defined as disease characterized by the rapid and uncontrolled growth of aberrant cells. Cancer cells can spread locally or through the bloodstream and lymphatic system to other parts of the body. Examples of cancers include but are not limited to, bone cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “ED₅₀” or “ED50” refers to the effective dose of a formulation that produces about 50% of the maximal effect in subjects that are administered that formulation.

As used herein, an “effective amount,” “therapeutically effective amount” or “pharmaceutically effective amount” of a compound is that amount of compound that is sufficient to provide a beneficial effect to the subject to which the compound is administered.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, a “patient” or “subject” may be a human or non-human mammal or a bird. Non-human mammals include, for example, livestock and pets, such as ovine, bovine, porcine, canine, feline and murine mammals. In certain other embodiments, the subject is human.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof. As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound include, but are not limited to, intravenous, oral, aerosol, parenteral, ophthalmic, pulmonary and topical administration.

The term “prevent,” “preventing,” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

The term “solvate,” as used herein, refers to a compound formed by solvation, which is a process of attraction and association of molecules of a solvent with molecules or ions of a solute. As molecules or ions of a solute dissolve in a solvent, they spread out and become surrounded by solvent molecules.

The term “treat,” “treating,” or “treatment,” as used herein, means reducing the frequency or severity with which symptoms of a disease or condition are experienced by a subject by virtue of administering an agent or compound to the subject.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “alkylene” by itself or as part of another substituent means, unless otherwise stated, a straight or branched hydrocarbon group having the number of carbon atoms designated (i.e., C₁-C₁₀ means one to ten carbon atoms) and includes straight, branched chain, or cyclic substituent groups, wherein the group has two open valencies. Examples include methylene, 1,2-ethylene, 1,1-ethylene, 1,1-propylene, 1,2-propylene and 1,3-propylene.

As used herein, the term “cycloalkyl,” by itself or as part of another substituent means, unless otherwise stated, a cyclic chain hydrocarbon having the number of carbon atoms designated (i.e., C3-C6 means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “alkenylene”, employed alone or in combination with other terms, means, unless otherwise stated, a stable mono-unsaturated or di-unsaturated straight chain or branched chain hydrocarbon group having the stated number of carbon atoms wherein the group has two open valencies.

As used herein, the term “alkynylene”, employed alone or in combination with other terms, means, unless otherwise stated, a stable straight chain or branched chain hydrocarbon group with a triple carbon-carbon bond, having the stated number of carbon atoms wherein the group has two open valencies.

As used herein, the term “substituted alkyl”, “substituted cycloalkyl”, “substituted alkenyl”, “substituted alkynyl”, “substituted alkylene”, “substituted alkenylene”, “substituted alkynylene”, “substituted heteroalkyl”, “substituted heteroalkenyl”, “substituted heteroalkynyl”, “substituted aryl”, “substituted heteroaryl” or “substituted heterocycloalkyl” means alkyl, cycloalkyl, alkenyl, alkynyl, alkylene, alkenylene, alkynylene, heteroalkyl, heteroalkenyl, heteroalkynyl, aryl, heteroaryl, or heterocycloalkyl as defined above, substituted by one, two or three substituents selected from the group consisting of C₁-C₁₀ alkyl, halogen, perhaloakyl, ═O, —OH, alkoxy, —NH₂, —N(CH₃)₂, —NH(CH₃)₂, phenyl, benzyl, (1-methyl-imidazol-2-yl), pyridin-2-yl, pyridin-3-yl, pyridin-4-yl, —C(═O)OH, —OC(═O) (C₁-C₄)alkyl, —C(═O)(C₁-C₄)alkyl, —C≡O, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heteroalkenyl” by itself or in combination with another term means, unless otherwise stated, a stable straight or branched chain monounsaturated or di unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “aromatic” refers to a carbocycle or heterocycle with one or more polyunsaturated rings and having aromatic character, i.e. having (4n+2) delocalized 7E (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain other embodiments, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3 dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group. Non-limiting examples of “substituted” groups include C₁-C₁₀ alkyl, halogen, perhaloakyl, ═O, —OH, alkoxy, —NH₂, —N(CH₃)₂, —NH(CH₃)₂, phenyl, benzyl, (1-methyl-imidazol-2-yl), pyridin-2-yl, pyridin-3-yl, pyridin-4-yl, —C(═O)OH, —OC(═O) (C₁-C₄)alkyl, —C(═O)(C₁-C₄)alkyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain other embodiments, the substituents vary in number between one and four. In other embodiments, the substituents vary in number between one and three. In yet other embodiments, the substituents vary in number between one and two. In yet other embodiments, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic, with straight being preferred. The term “substituted heterocycle” and “substituted heteroaryl” as used herein refers to a heterocycle or heteroaryl group having one or more substituents including halogen, CN, OH, NO₂, amino, alkyl, cycloalkyl, carboxyalkyl (C(O)Oalkyl), trifluoroalkyl such as CF₃, aryloxy, alkoxy, aryl, or heteroaryl. A substituted heterocycle or heteroaryl group may have 1, 2, 3, or 4 substituents.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following abbreviations are used herein: Boc, tert-Butyloxycarbonyl; (Bpin)₂, Bis(pinacolato)diboron; Cs₂CO₃, Cesium carbonate; DCM, Dichloromethane; DEA, Diethylamine; DIPEA, N,N-Diisopropylethylamine; DMF, Dimethylformamide; DMSO, Dimethyl sulfoxide; ER, endoplasmic reticulum; ERAD, endoplasmic reticulum-associated degradation; EtOAc, Ethyl acetate; EtOH, Ethanol; Et₂O, Diethyl ether; HPLC, High-performance liquid chromatography; IPA, 2-Propanol; KOAc, Potassium acetate; LC-MS, Liquid chromatography-mass spectrometry; MDAP, Mass-directed automated purification; MeCN, Acetonitrile; MeOH, Methanol; MgSO₄, Magnesium sulfate; Na₂SO₄, Sodium sulfate; NBS, N-bromosuccinimide; NIS, N-iodosuccinimide; Pd(dppf)Cl₂.DCM, [1,1′-Bis(diphenylphosphino)ferrocenel-dichloropalladium(II) DCM complex; Ph, phenyl; Ph₃P, triphenylphosphine; RP, Retinitis pigmentosa; RT or rt, Room temperature; Rₜ, Retention time; SCX-2, Biotage Isolute—strong cationic ion-exchange resin; TEA, trimethylamine; TFA, trifluoroacetic acid; THF, tetrahydrofuran; TLC, thin layer chromatography; UPLC, Ultra-high performance liquid chromatography; UPR, unfolded protein response.

### Compounds and Compositions

The invention includes a compound of formula (I), or a salt, solvate, enantiomer, diastereoisomer, isotopologue, or tautomer thereof:

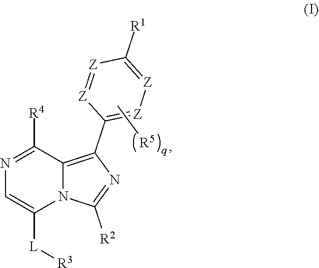

wherein:

R¹ is

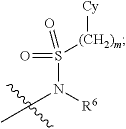

R² is selected from the group consisting of H, methyl, ethyl, propyl, CF₃, CHF₂, cyclopropyl, 1-methylcyclopropyl, isopropyl, tert-butyl, and C₃-C₈ cycloalkyl;

L is selected from the group consisting of a bond, —CH₂—, —C(═O)—, —C(═O)NH, and —C(═O)N(C₁-C₆ alkyl);

R³ is selected from the group consisting of optionally substituted C₁-C₈ alkyl, optionally substituted C₃-C₈ cycloalkyl, optionally substituted C₂-C₈ alkenyl, optionally substituted C₂-C₈ alkynyl, optionally substituted C₁-C₈ heteroalkyl (such as, but not limited to, N-linked C₁-C₈ aminoalkyl), optionally substituted C₃-C₈ heterocycloalkyl, optionally substituted C₂-C₈ heteroalkenyl, optionally substituted benzyl, optionally substituted C₂-C₈ cycloheteroalkenyl, optionally substituted heterocyclyl, optionally substituted aryl, and optionally substituted heteroaryl (such as, but not limited to, imidazolyl or pyrazolyl);

R⁴ is selected from the group consisting of —NH₂ and —NHR⁸;

each instance of R⁵ is independently selected from the group consisting of halide, —OH, C₁-C₆ alkoxy, optionally substituted phenyl, optionally substituted C₁-C₆ alkyl, optionally substituted C₁-C₆ alkoxy, and optionally substituted heterocycloalkyl;

R⁶ is selected from the group consisting of H and optionally substituted C₁-C₆ alkyl;

R⁸ is optionally substituted C₁-C₃ alkyl;

Cy is selected from the group consisting of aryl, heteroaryl, C₃-C₁₀ cycloalkyl, C₃-C₁₀ cycloalkenyl, C₃-C₁₀ heterocycloalkyl, C₃-C₁₀ heterocycloalkenyl, polycyclic aryl, polycyclic heteroaryl, polycyclic C₃-C₁₀ cycloalkyl, polycyclic C₃-C₁₀ cycloalkenyl, polycyclic C₃-C₁₀ heterocycloalkyl, and polycyclic C₃-C₁₀ heterocycloalkenyl;
wherein Cy is substituted with 0 to ‘n’ instances of X, each instance of X being independently selected from the group consisting of H, OH, halide, nitrile, optionally substituted C₁-C₆ alkyl, C₁-C₆ haloalkyl, optionally substituted C₁-C₆ alkoxy, optionally substituted aryl (such as, but not limited, phenyl), optionally substituted heteroaryl, and

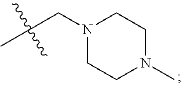 [SMILES: *CN1CCN(C)CC1]

each instance of Z, if present, is independently selected from the group consisting of CH and N, with the proviso that there are 0-3 N ring atoms per ring;

m is an integer selected from the group consisting of 0, 1, 2, 3, and 4;

n is an integer selected from the group consisting of 0, 1, 2, 3, 4, and 5; and

q is an integer selected from the group consisting of 0, 1, 2, 3, and 4.

In certain embodiments, an optionally substituted group is unsubstituted. In other embodiments, an optionally substituted group is substituted with at least substituent contemplated herein.

In certain embodiments, each occurrence of optionally substituted alkyl, alkenyl, alkynyl, heteroalkyl, heterocycloalkyl, heteroalkenyl, benzyl, heterocyclyl, or cycloalkyl is independently optionally substituted with at least one substituent selected from the group consisting of C₁-C₆ alkyl, halo, —OR^{a}, optionally substituted phenyl, optionally substituted heteroaryl, optionally substituted heterocyclyl, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

In certain embodiments, each occurrence of optionally substituted aryl or heteroaryl is independently optionally substituted with at least one substituent selected from the group consisting of C₁-C₆ alkyl, C₁-C₆ haloalkyl, C₁-C₆ haloalkoxy, halo, —CN, —OR^{b}, —N(R^{b})(R^{b}), —NO₂, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl.

In certain embodiments, each occurrence of optionally substituted aryl or heteroaryl is independently optionally substituted with at least one substituent selected from the group consisting of C₁-C₆ alkyl, C₁-C₆ haloalkyl, C₁-C₆ haloalkoxy, halo, —CN, —N(R^{c})(R^{c}), and C₁-C₆ alkoxycarbonyl, wherein each occurrence of R^{c} is independently H, C₁-C₆ alkyl, or C₃-C₈ cycloalkyl.

In certain embodiments, R¹ is

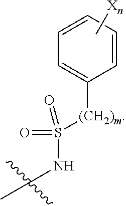

In certain embodiments, R¹ is

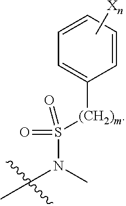

In certain embodiments, R¹ is

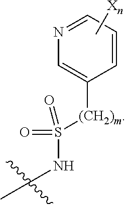

In certain embodiments, R¹ is

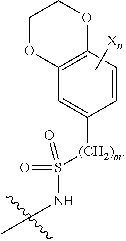

In certain embodiments, R¹ is

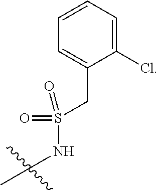 [SMILES: *NS(=O)(=O)Cc1ccccc1Cl]

In certain embodiments, R¹ is

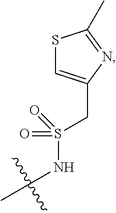 [SMILES: *NS(=O)(=O)Cc1csc(C)n1]

In certain embodiments, R¹ is

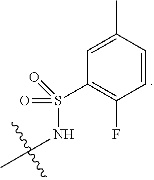 [SMILES: *NS(=O)(=O)c1cc(C)ccc1F]

In certain embodiments, R¹ is

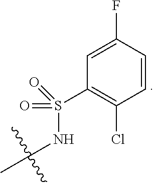 [SMILES: *NS(=O)(=O)c1cc(F)ccc1Cl]

In certain embodiments, R¹ is

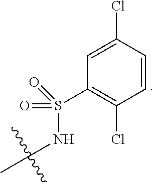 [SMILES: *NS(=O)(=O)c1cc(Cl)ccc1Cl]

In certain embodiments, R¹ is

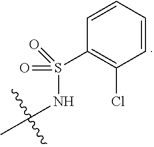 [SMILES: *NS(=O)(=O)c1ccccc1Cl]

In certain embodiments, R¹ is

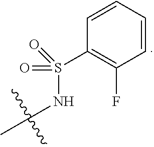 [SMILES: *NS(=O)(=O)c1ccccc1F]

In certain embodiments, R¹ is

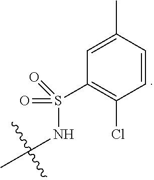 [SMILES: *NS(=O)(=O)c1cc(C)ccc1Cl]

In certain embodiments, R¹ is

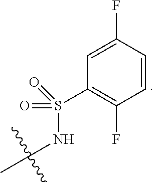 [SMILES: *NS(=O)(=O)c1cc(F)ccc1F]

In certain embodiments, R¹ is

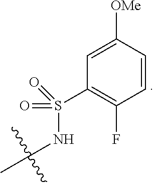 [SMILES: *NS(=O)(=O)c1cc(OC)ccc1F]

In certain embodiments, R¹ is

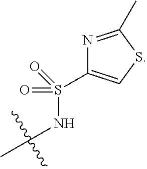 [SMILES: *NS(=O)(=O)c1csc(C)n1]

In certain embodiments, R¹ is

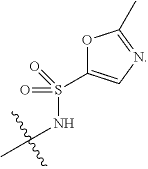 [SMILES: *NS(=O)(=O)c1cnc(C)o1]

In certain embodiments, R¹ is

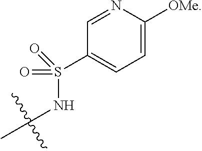 [SMILES: *NS(=O)(=O)c1ccc(OC)nc1]

In certain embodiments, R¹ is

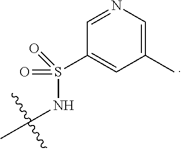 [SMILES: *NS(=O)(=O)c1cncc(C)c1]

In certain embodiments, R¹ is

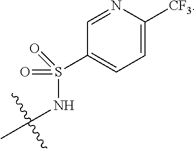 [SMILES: *NS(=O)(=O)c1ccc(C(F)(F)F)nc1]

In certain embodiments, R¹ is

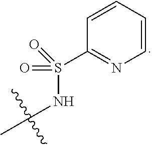 [SMILES: *NS(=O)(=O)c1ccccn1]

In certain embodiments, R¹ is

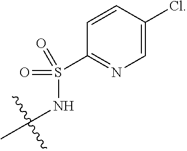 [SMILES: *NS(=O)(=O)c1ccc(Cl)cn1]

In certain embodiments, R² is methyl. In certain embodiments, R² is ethyl. In certain embodiments, R² is isopropyl. In other embodiments, R² is cyclopropyl.

In certain embodiments, R³ is

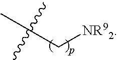

In certain embodiments, R³ is

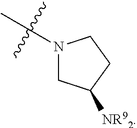

In certain embodiments, R³ is

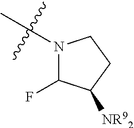

In certain embodiments, R³ is

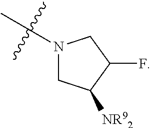

In certain embodiments, R³ is

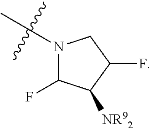

In certain embodiments, R³ is

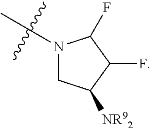

In certain embodiments, R³ is

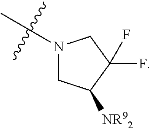

In certain embodiments, R³ is

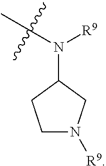

In certain embodiments, R³ is

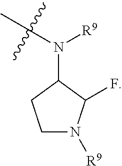

In certain embodiments, R³ is

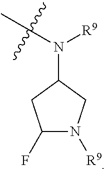

In certain embodiments, R³ is

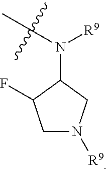

In certain embodiments, R³ is

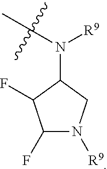

In certain embodiments, R³ is

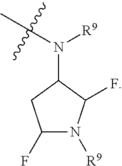

In certain embodiments, R³ is

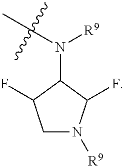

In certain embodiments, R³ is

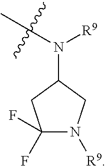

In certain embodiments, R³ is

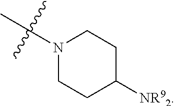

In certain embodiments, R³ is

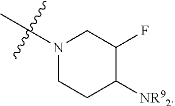

In certain embodiments, R³ is

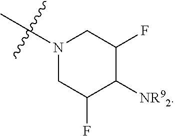

In certain embodiments, R³ is

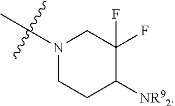

In certain embodiments, R³ is

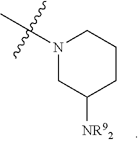

In certain embodiments, R³ is

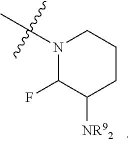

In certain embodiments, R³ is

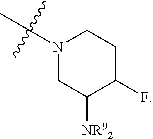

In certain embodiments, R³ is

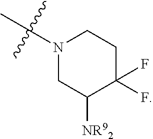

In certain embodiments, R³ is

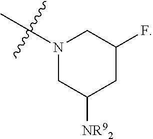

In certain embodiments, R³ is

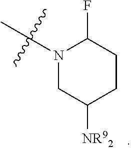

In certain embodiments, R³ is

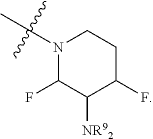

In certain embodiments, R³ is

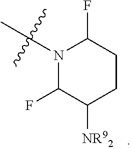

In certain embodiments, R³ is

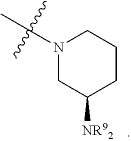

In certain embodiments, R³ is

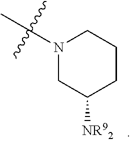

In certain embodiments, R³ is

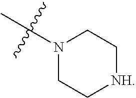 [SMILES: *N1CCNCC1]

In certain embodiments, R³ is

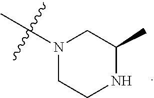 [SMILES: *N1CCN[C@H](C)C1]

In certain embodiments, R³ is

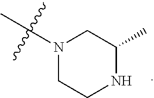 [SMILES: *N1CCN[C@@H](C)C1]

In certain embodiments, R³ is

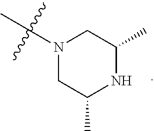 [SMILES: *N1C[C@@H](C)N[C@@H](C)C1]

In certain embodiments, R³ is

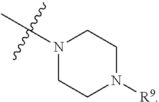

In certain embodiments, R³ is

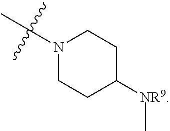

In certain embodiments, R³ is

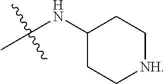 [SMILES: *NC1CCNCC1]

In certain embodiments, R³ is

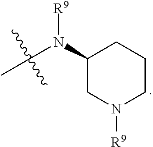

In certain embodiments, R³ is

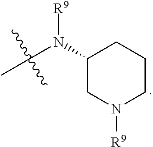

In certain embodiments, R³ is

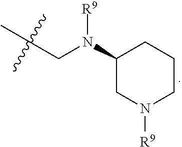

In certain embodiments, R³ is

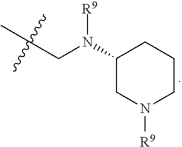

In certain embodiments, R³ is

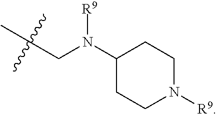

In certain embodiments, R³ is

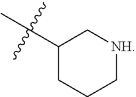 [SMILES: *C1CCCNC1]

In certain embodiments, R³ is

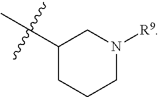

In certain embodiments, R³ is

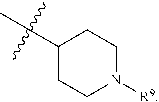

In certain embodiments, R³ is

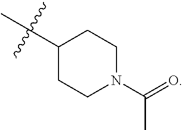 [SMILES: *C1CCN(C(C)=O)CC1]

In certain embodiments, R³ is

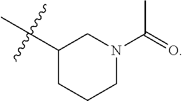 [SMILES: *C1CCCN(C(C)=O)C1]

In certain embodiments, R³ is

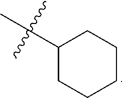 [SMILES: *C1CCCCC1]

In certain embodiments, R³ is

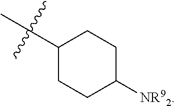

In certain embodiments, R³ is

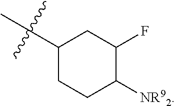

In certain embodiments, R³ is

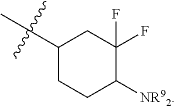

In certain embodiments, R³ is

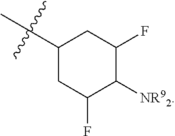

In certain embodiments, R³ is

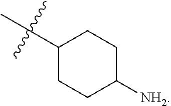 [SMILES: *C1CCC(N)CC1]

In certain embodiments, R³ is

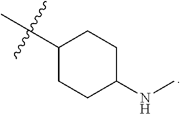 [SMILES: *C1CCC(NC)CC1]

In certain embodiments, R³ is

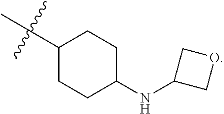 [SMILES: *C1CCC(NC2COC2)CC1]

In certain embodiments, R³ is

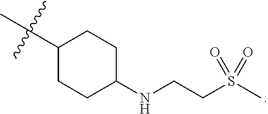 [SMILES: *C1CCC(NCCS(C)(=O)=O)CC1]

In certain embodiments, R³ is

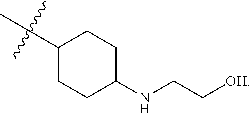 [SMILES: *C1CCC(NCCO)CC1]

certain embodiments, R³ is

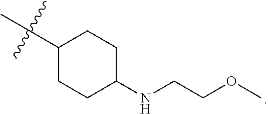 [SMILES: *C1CCC(NCCOC)CC1]

In certain embodiments, R³ is

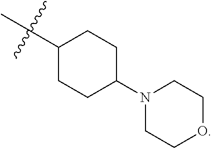 [SMILES: *C1CCC(N2CCOCC2)CC1]

In certain embodiments, R³ is

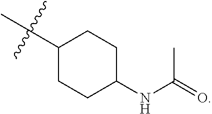 [SMILES: *C1CCC(NC(C)=O)CC1]

In certain embodiments, R³ is

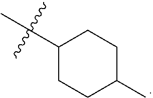 [SMILES: *C1CCC(C)CC1]

In certain embodiments, R³ is

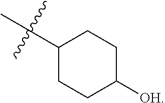 [SMILES: *C1CCC(O)CC1]

In certain embodiments, R³ is

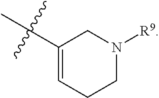

In certain embodiments, R³ is

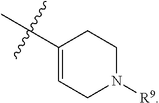

In certain embodiments, R³ is

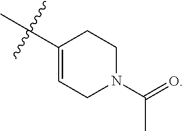 [SMILES: *C1=CCN(C(C)=O)CC1]

In certain embodiments, R³ is

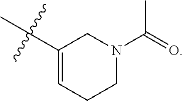 [SMILES: *C1=CCCN(C(C)=O)C1]

In certain embodiments, R³ is

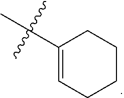 [SMILES: *C1=CCCCC1]

In certain embodiments, R³ is

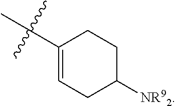

In certain embodiments, R³ is

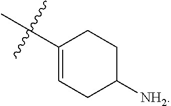 [SMILES: *C1=CCC(N)CC1]

In certain embodiments, R³ is

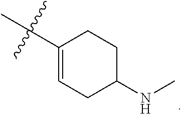 [SMILES: *C1=CCC(NC)CC1]

In certain embodiments, R³ is

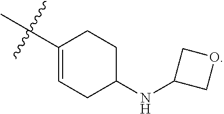 [SMILES: *C1=CCC(NC2COC2)CC1]

In certain embodiments, R³ is

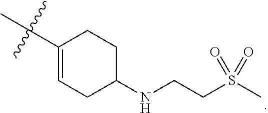 [SMILES: *C1=CCC(NCCS(C)(=O)=O)CC1]

In certain embodiments, R³ is

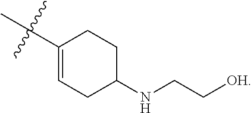 [SMILES: *C1=CCC(NCCO)CC1]

certain embodiments, R³ is

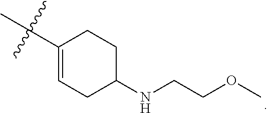 [SMILES: *C1=CCC(NCCOC)CC1]

In certain embodiments, R³ is

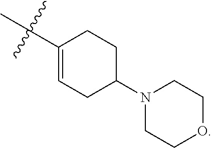 [SMILES: *C1=CCC(N2CCOCC2)CC1]

In certain embodiments, R³ is

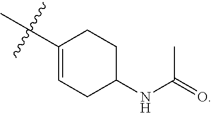 [SMILES: *C1=CCC(NC(C)=O)CC1]

In certain embodiments, R³ is

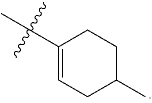 [SMILES: *C1=CCC(C)CC1]

In certain embodiments, R³ is

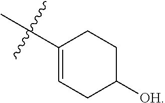 [SMILES: *C1=CCC(O)CC1]

In certain embodiments, R³ is

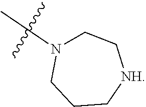 [SMILES: *N1CCCNCC1]

In certain embodiments, R³ is

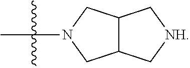 [SMILES: *N1CC2CNCC2C1]

In certain embodiments, R³ is

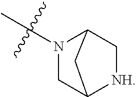 [SMILES: *N1CC2CC1CN2]

In certain embodiments, R³ is

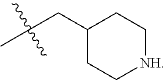 [SMILES: *CC1CCNCC1]

In certain embodiments, R³ is

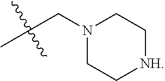 [SMILES: *CN1CCNCC1]

In certain embodiments, R³ is

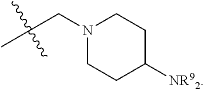

In certain embodiments, R³ is

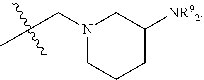

In certain embodiments, R³ is

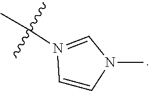

In certain embodiments, R³ is

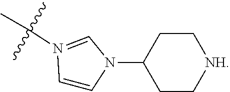

In certain embodiments, R³ is

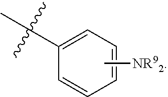

In certain embodiments, R³ is

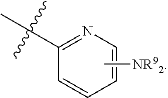

In certain embodiments, R³ is

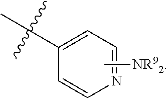

In certain embodiments, R³ is

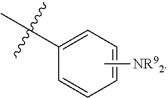

In certain embodiments, p is 0. In certain embodiments, p is 1. In certain embodiments, p is 2. In certain embodiments, p is 3. In certain embodiments, p is 4. In certain embodiments, p is 5.

In certain embodiments, each occurrence of R⁹ is independently selected from the group consisting of H, oxetanyl, C₁-C₆ hydroxyalkyl, C₁-C₆ haloalkyl, C₁-C₆ carboxamido alkyl, C₁-C₆ carboxy alkyl, C₁-C₆ carboxy(C₁-C₆)alkyl alkyl, C₁-C₆ cyano alkyl, and C₁-C₆ sulfonylalkyl.

In certain embodiments, L=bond, and R³ is

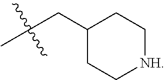 [SMILES: *CC1CCNCC1]

In certain embodiments, L=bond, and R³ is

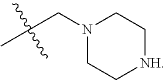 [SMILES: *CN1CCNCC1]

In certain embodiments, L=bond, and R³ is

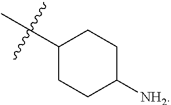 [SMILES: *C1CCC(N)CC1]

In certain embodiments, L=bond, and R³ is

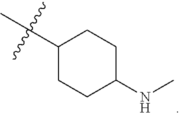 [SMILES: *C1CCC(NC)CC1]

In certain embodiments, L=bond, and R³ is

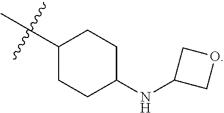 [SMILES: *C1CCC(NC2COC2)CC1]

In certain embodiments, L=bond, and R³ is

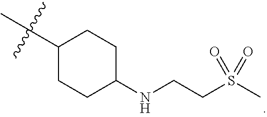 [SMILES: *C1CCC(NCCS(C)(=O)=O)CC1]

In certain embodiments, L=bond, and R³ is

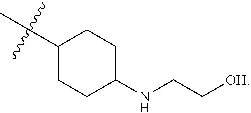 [SMILES: *C1CCC(NCCO)CC1]

In certain embodiments, L=bond, and R³ is

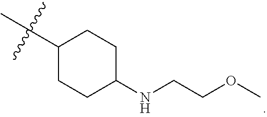 [SMILES: *C1CCC(NCCOC)CC1]

In certain embodiments, L=bond, and R³ is

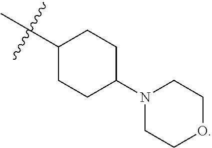 [SMILES: *C1CCC(N2CCOCC2)CC1]

In certain embodiments, L=bond, and R³ is

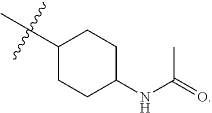 [SMILES: *C1CCC(NC(C)=O)CC1]

In certain embodiments, L=bond, and R³ is

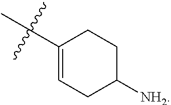 [SMILES: *C1=CCC(N)CC1]

In certain embodiments, L=bond, and R³ is

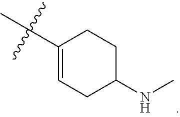 [SMILES: *C1=CCC(NC)CC1]

In certain embodiments, L=bond, and R³ is

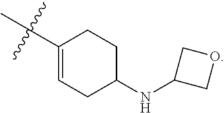 [SMILES: *C1=CCC(NC2COC2)CC1]

In certain embodiments, L=bond, and R³ is

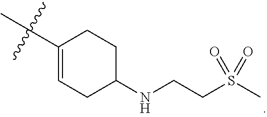 [SMILES: *C1=CCC(NCCS(C)(=O)=O)CC1]

In certain embodiments, L=bond, and R³ is

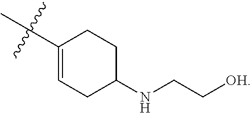 [SMILES: *C1=CCC(NCCO)CC1]

In certain embodiments, L=bond, and R³ is

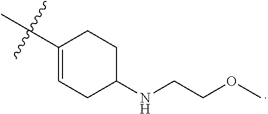 [SMILES: *C1=CCC(NCCOC)CC1]

In certain embodiments, L=bond, and R³ is

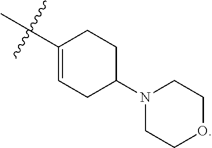 [SMILES: *C1=CCC(N2CCOCC2)CC1]

In certain embodiments, L=bond, and R³ is

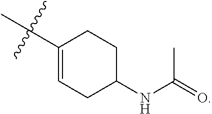 [SMILES: *C1=CCC(NC(C)=O)CC1]

In certain embodiments, R⁴ is —NH₂.

In certain embodiments, R⁵, if present, is a halogen. In other embodiments, q=1 and R⁵ is F.

In certain embodiments, each occurrence of R⁹ is independently selected from the group consisting of H, oxetanyl, C₁-C₆ hydroxyalkyl, C₁-C₆ haloalkyl, C₁-C₆ carboxamido alkyl, C₁-C₆ carboxy alkyl, C₁-C₆ carboxy(C₁-C₆)alkyl alkyl, C₁-C₆ cyano alkyl, and C₁-C₆ sulfonylalkyl.

In certain embodiments, each occurrence of R⁹ is independently selected from the group consisting of: H, oxetanyl, C₁-C₈ alkyl,

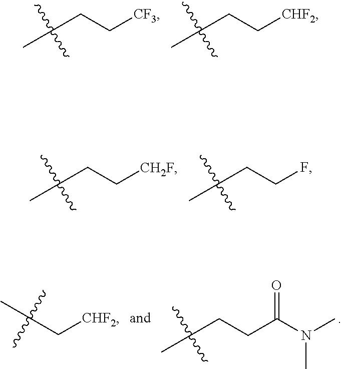

In certain embodiments, the compound is

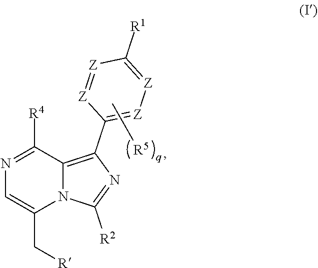

wherein R′ is R³ as defined elsewhere herein.

In certain embodiments, R′ is optionally substituted heterocyclyl. In certain embodiments, R′ is optionally substituted —NH-(optionally substituted heterocyclyl). In certain embodiments, R′ is optionally substituted —N(C₁-C₆ alkyl)-(optionally substituted heterocyclyl). In certain embodiments, R′ is

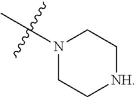 [SMILES: *N1CCNCC1]

In certain embodiments, R′ is

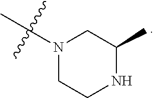 [SMILES: *N1CCN[C@H](C)C1]

In certain embodiments, R′ is

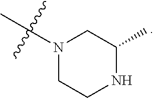 [SMILES: *N1CCN[C@@H](C)C1]

In certain embodiments, R′ is

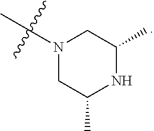 [SMILES: *N1C[C@@H](C)N[C@@H](C)C1]

In certain embodiments, R′ is

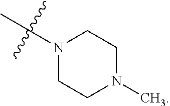 [SMILES: *N1CCN(C)CC1]

In certain embodiments, R′ is

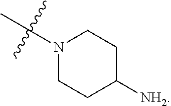 [SMILES: *N1CCC(N)CC1]

In certain embodiments, R′ is

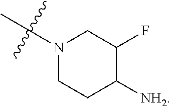 [SMILES: *N1CCC(N)C(F)C1]

In certain embodiments, R′ is

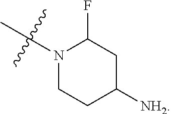 [SMILES: *N1CCC(N)CC1F]

In certain embodiments, R′ is

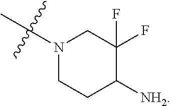 [SMILES: *N1CCC(N)C(F)(F)C1]

In certain embodiments, R′ is

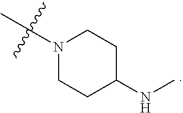 [SMILES: *N1CCC(NC)CC1]

In certain embodiments, R′ is

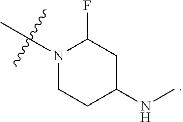 [SMILES: *N1CCC(NC)CC1F]

In certain embodiments, R′ is

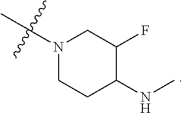 [SMILES: *N1CCC(NC)C(F)C1]

In certain embodiments, R′ is

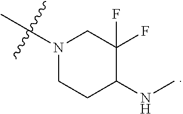 [SMILES: *N1CCC(NC)C(F)(F)C1]

In certain embodiments, R′ is

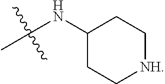 [SMILES: *NC1CCNCC1]

In certain embodiments, R′ is

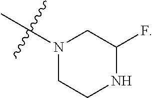 [SMILES: *N1CCNC(F)C1]

In certain embodiments, R′ is

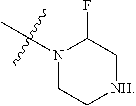 [SMILES: *N1CCNCC1F]

In certain embodiments, R′ is

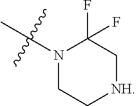 [SMILES: *N1CCNCC1(F)F]

In certain embodiments, R′ is

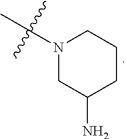 [SMILES: *N1CCCC(N)C1]

In certain embodiments, R′ is

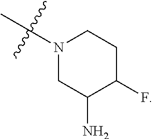 [SMILES: *N1CCC(F)C(N)C1]

In certain embodiments, R′ is

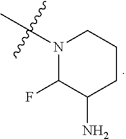 [SMILES: *N1CCCC(N)C1F]

In certain embodiments, R′ is

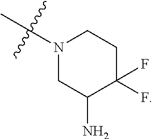 [SMILES: *N1CCC(F)(F)C(N)C1]

In certain embodiments, R′ is

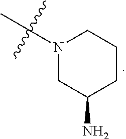 [SMILES: *N1CCC[C@@H](N)C1]

In certain embodiments, R′ is

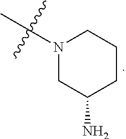 [SMILES: *N1CCC[C@H](N)C1]

In certain embodiments, R′ is

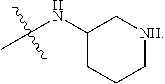 [SMILES: *NC1CCCNC1]

In certain embodiments, R′ is

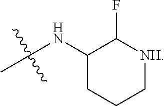 [SMILES: *NC1CCCNC1F]

In certain embodiments, R′ is

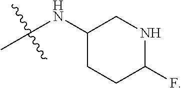 [SMILES: *NC1CCC(F)NC1]

In certain embodiments, R′ is

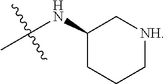 [SMILES: *N[C@@H]1CCCNC1]

In certain embodiments, R′ is

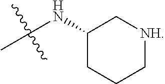 [SMILES: *N[C@H]1CCCNC1]

In certain embodiments, R′ is

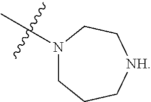 [SMILES: *N1CCCNCC1]

In certain embodiments, R′ is

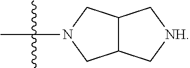 [SMILES: *N1CC2CNCC2C1]

In certain embodiments, the compound is

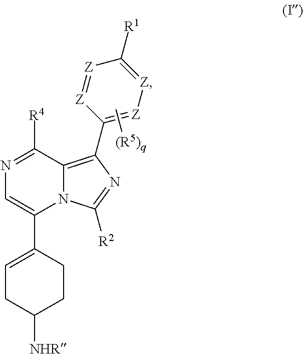

wherein R″ is H or optionally substituted C₁-C₆ alkyl.

In certain embodiments, R″ is H. In certain embodiments, R″ is optionally substituted C₁-C₆ alkyl.

In certain embodiments, the compound is

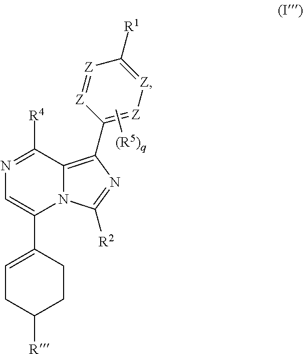

wherein R′″ in (I′″) is selected from the group consisting of —OH, C₁-C₆ alkoxy, —NH₂, —NH(C₁-C₆ alkyl), —N(C₁-C₆ alkyl)(C₁-C₆ alkyl), and —NH(oxetanyl), wherein each C₁-C₆ alkyl is optionally substituted with at least one independently selected from the group consisting of halogen, —C(═O)NH₂, —C(═O)N(C₁-C₆ alkyl), —C(═O)N(C₁-C₆ alkyl)(C₁-C₆ alkyl), —OH, C₁-C₆ alkoxy, and C₁-C₆ sulfonylalkyl.

In certain embodiments, R′″ is H. In certain embodiments, R′″ is —OH. In certain embodiments, R′″ is —NH₂. In certain embodiments, R′″ is —NHCH₃. In certain embodiments, R′″ is —N(CH₃)₂. In certain embodiments, R′″ is —NHCH₂CH₂F. In certain embodiments, R′″ is —N(Me)CH₂CH₂F. In certain embodiments, R′″ is —NHCH₂CHF₂. In certain embodiments, R′″ is —N(Me)CH₂CHF₂. In certain embodiments, R′″ is —NHCH₂CF₃. In certain embodiments, R′″ is —N(Me)CH₂CF₃. In certain embodiments, R′″ is —NHCH₂CH₂CF₃. In certain embodiments, R′″ is —N(Me)CH₂CH₂CF₃. In certain embodiments, R′″ is —NHCH₂CH₂C(═O)NMe₂. In certain embodiments, R′″ is —N(Me)CH₂CH₂C(═O)NMe₂. In certain embodiments, R′″ is —NHCH₂CH₂C(═O)NH₂. In certain embodiments, R′″ is —N(Me)CH₂CH₂C(═O)NH₂. In certain embodiments, R′″ is —NHCH₂CH₂C(═O)NHMe. In certain embodiments, R′″ is —SO₂(C₁-C₆ alkyl). In certain embodiments, R′″ is —N(Me)CH₂CH₂C(═O)NHMe₂. In certain embodiments, R′″ is

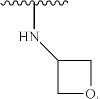 [SMILES: *NC1COC1]

In certain embodiments, the compound is

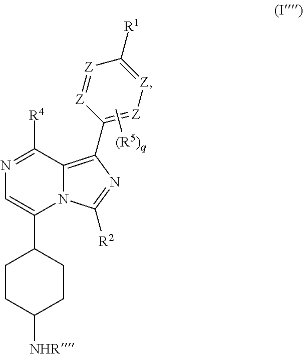

wherein R″″ is H or optionally substituted C₁-C₆ alkyl.

In certain embodiments, R″″ is H. In certain embodiments, R″″ is optionally substituted C₁-C₆ alkyl.

In certain embodiments, the compound is

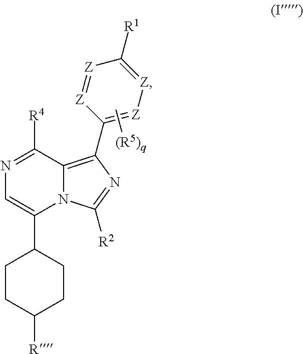

wherein R″″ in (I″″) is selected from the group consisting of —OH, C₁-C₆ alkoxy, —NH₂, —NH(C₁-C₆ alkyl), —N(C₁-C₆ alkyl)(C₁-C₆ alkyl), and —NH(oxetanyl), wherein each C₁-C₆ alkyl is optionally substituted with at least one independently selected from the group consisting of halogen, —C(═O)NH₂, —C(═O)N(C₁-C₆ alkyl), —C(═O)N(C₁-C₆ alkyl)(C₁-C₆ alkyl), —OH, C₁-C₆ alkoxy, and C₁-C₆ sulfonylalkyl.

In certain embodiments, R″″ is H. In certain embodiments, R″″ is —OH. In certain embodiments, R″″ is —NH₂. In certain embodiments, R″″ is —NHCH₃. In certain embodiments, R″″ is —N(CH₃)₂. In certain embodiments, R″″ is —NHCH₂CH₂F. In certain embodiments, R″″ is —N(Me)CH₂CH₂F. In certain embodiments, R″″ is —NHCH₂CHF₂. In certain embodiments, R″″ is —N(Me)CH₂CHF₂. In certain embodiments, R″″ is —NHCH₂CF₃. In certain embodiments, R″″ is —N(Me)CH₂CF₃. In certain embodiments, R″″ is —NHCH₂CH₂CF₃. In certain embodiments, R″″ is —N(Me)CH₂CH₂CF₃. In certain embodiments, R″″ is —NHCH₂CH₂C(═O)NMe₂. In certain embodiments, R″″ is —N(Me)CH₂CH₂C(═O)NMe₂. In certain embodiments, R″″ is —NHCH₂CH₂C(═O)NH₂. In certain embodiments, R″″ is —N(Me)CH₂CH₂C(═O)NH₂. In certain embodiments, R″″ is —NHCH₂CH₂C(═O)NHMe. In certain embodiments, R″″ is —SO₂(C₁-C₆ alkyl). In certain embodiments, R″″ is —N(Me)CH₂CH₂C(═O)NHMe₂. In certain embodiments, R″″ is

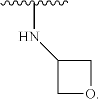 [SMILES: *NC1COC1]

In certain embodiments, the compound is

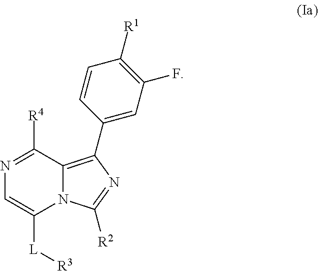

In certain embodiments, the compound is

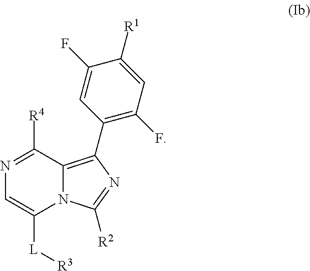

In certain embodiments, the compound is

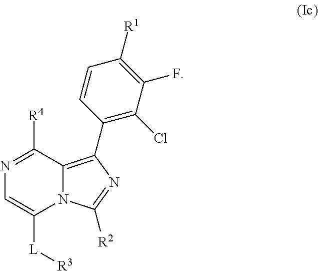

In certain embodiments, the compound is

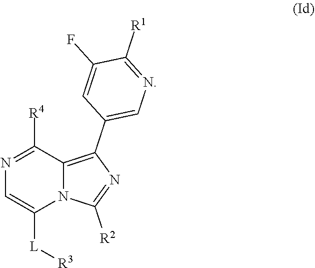

In certain embodiments, the compound is

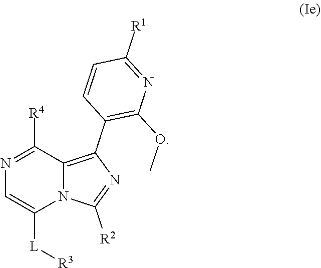

In certain embodiments, the compound is

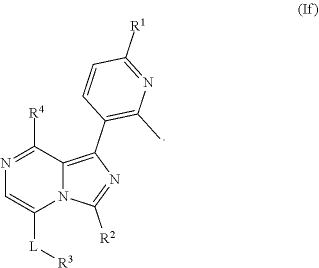

In certain embodiments, the compound is

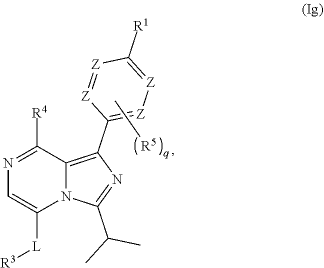

wherein R² is isopropyl.

In certain embodiments, the compound is

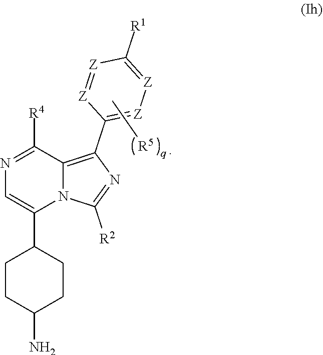

In certain embodiments, the compound is

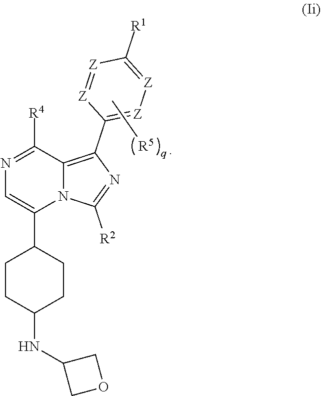

In certain embodiments, the compound is

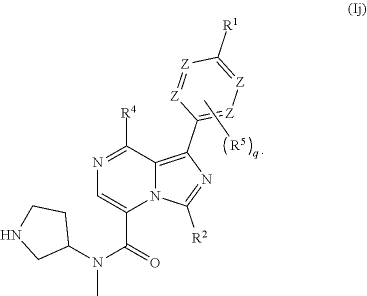

In certain embodiments, the compound is

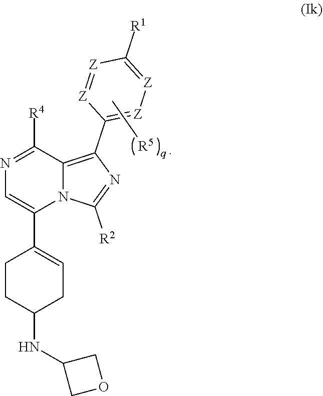

In certain embodiments, the compound is

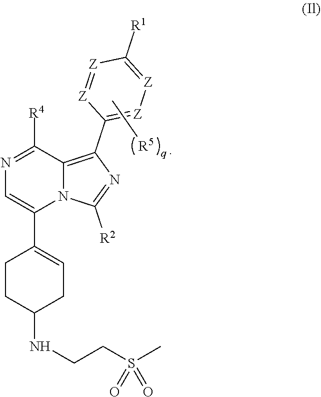

In certain embodiments, the compound is

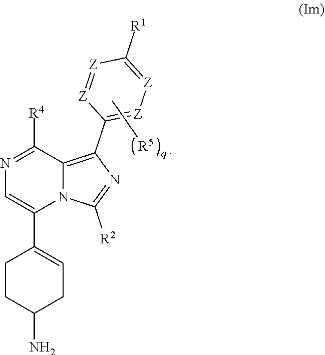

In certain embodiments, the compound is

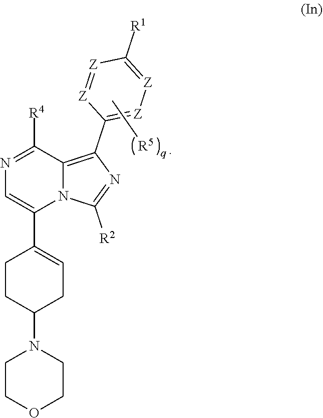

In certain embodiments, the compound is selected from the group consisting of:

### Example 1

### N-(4-(8-Amino-5-((1s,4s)-4-aminocyclohexyl)-3-isopropylimidazo[1,5-a]pyrazin-1-yl)-2-fluorophenyl)-2-chlorobenzenesulfonamide (Cis Isomer)

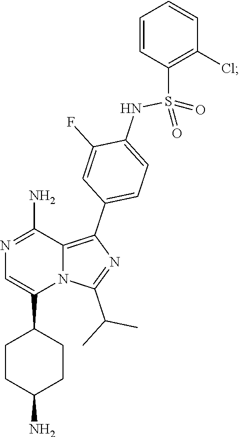 [SMILES: CC(C)c1nc(-c2ccc(NS(=O)(=O)c3ccccc3Cl)c(F)c2)c2c(N)ncc([C@H]3CC[C@@H](N)CC3)n12]

### Example 2

### N-(4-(8-Amino-5-((1r,4r)-4-aminocyclohexyl)-3-isopropylimidazo[1,5-a]pyrazin-1-yl)-2-fluorophenyl)-2-chlorobenzenesulfonamide (Trans Isomer)

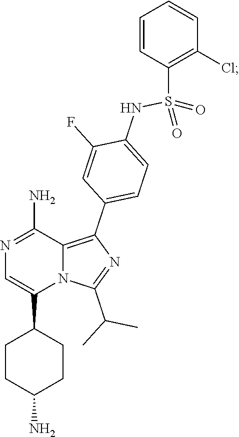 [SMILES: CC(C)c1nc(-c2ccc(NS(=O)(=O)c3ccccc3Cl)c(F)c2)c2c(N)ncc([C@H]3CC[C@H](N)CC3)n12]

### Example 3

### N-(4-(8-Amino-3-isopropyl-5-((1s,4s)-4-(oxetan-3-ylamino) cyclohexyl)imidazo[1,5-a]pyrazin-1-yl)-2-fluorophenyl)-2-chlorobenzenesulfonamide (Cis Isomer)

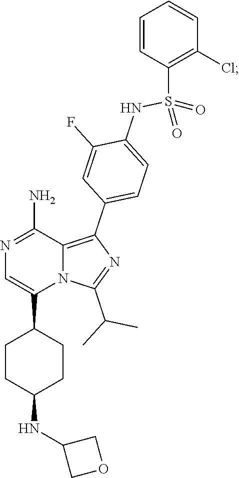 [SMILES: CC(C)c1nc(-c2ccc(NS(=O)(=O)c3ccccc3Cl)c(F)c2)c2c(N)ncc([C@H]3CC[C@@H](NC4COC4)CC3)n12]

### Example 4

### N-(4-(8-Amino-3-isopropyl-5-((1r,4r)-4-(oxetan-3-ylamino) cyclohexyl)imidazo[1,5-a]pyrazin-1-yl)-2-fluorophenyl)-2-chloro benzene sulfonamide (Trans Isomer)

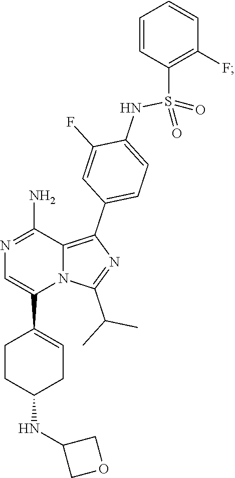

### Example 5

### 8-Amino-1-(4-(((2-chlorophenyl)methyl)sulfonamido)-3-fluorophenyl)-3-isopropyl-N-(pyrrolidin-3-yl)imidazo[1,5-a]pyrazine-5-carboxamide

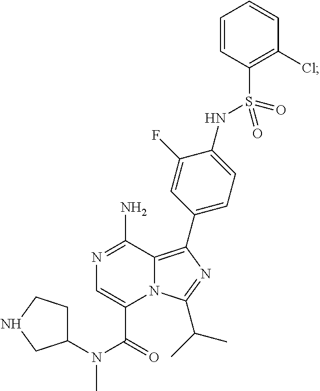 [SMILES: CC(C)c1nc(-c2ccc(NS(=O)(=O)c3ccccc3Cl)c(F)c2)c2c(N)ncc(C(=O)N(C)C3CCNC3)n12]

### Example 6

### N-(4-(8-Amino-3-isopropyl-5-(4-(oxetan-3-ylamino)cyclohex-1-en-1-yl)imidazo[1,5-a]pyrazin-1-yl)-2-fluorophenyl)-2-chlorobenzenesulfonamide

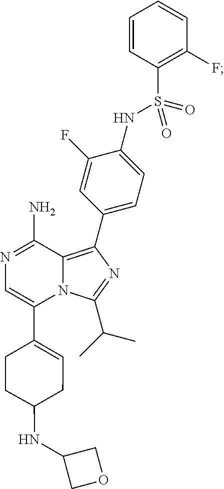 [SMILES: CC(C)c1nc(-c2ccc(NS(=O)(=O)c3ccccc3F)c(F)c2)c2c(N)ncc(C3=CCC(NC4COC4)CC3)n12]

### Example 7

### N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-fluorobenzenesulfonamide

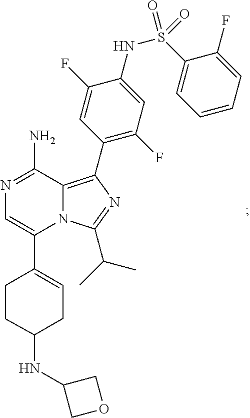 [SMILES: CC(C)c1nc(-c2cc(F)c(NS(=O)(=O)c3ccccc3F)cc2F)c2c(N)ncc(C3=CCC(NC4COC4)CC3)n12]

### Example 8

### N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-chlorobenzenesulfonamide

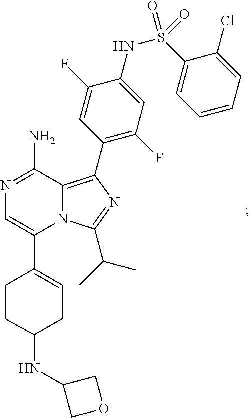 [SMILES: CC(C)c1nc(-c2cc(F)c(NS(=O)(=O)c3ccccc3Cl)cc2F)c2c(N)ncc(C3=CCC(NC4COC4)CC3)n12]

### Example 9

### N-(4-(8-Amino-3-isopropyl-5-(4-(oxetan-3-ylamino)cyclohex-1-en-1-yl) imidazo[1,5-a]pyrazin-1-yl)-3-chloro-2-fluorophenyl)-2-fluorobenzenesulfonamide

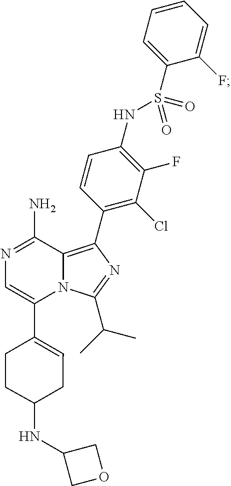 [SMILES: CC(C)c1nc(-c2ccc(NS(=O)(=O)c3ccccc3F)c(F)c2Cl)c2c(N)ncc(C3=CCC(NC4COC4)CC3)n12]

### Example 10

### N-(5-(8-Amino-3-isopropyl-5-(4-(oxetan-3-ylamino)cyclohex-1-en-1-yl) imidazo[1,5-a]pyrazin-1-yl)-3-fluoropyridin-2-yl)-2-chlorobenzenesulfonamide

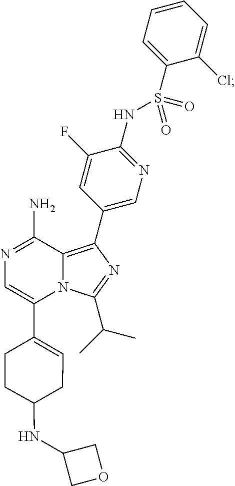 [SMILES: CC(C)c1nc(-c2cnc(NS(=O)(=O)c3ccccc3Cl)c(F)c2)c2c(N)ncc(C3=CCC(NC4COC4)CC3)n12]

### Example 11

### N-(4-(8-Amino-3-isopropyl-5-(4-(oxetan-3-ylamino)cyclohex-1-en-1-yl) imidazo[1,5-a]pyrazin-1-yl)-2-fluorophenyl)-6-methoxypyridine-3-sulfonamide

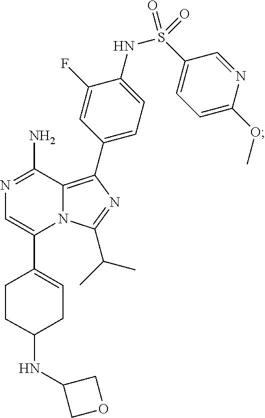 [SMILES: COc1ccc(S(=O)(=O)Nc2ccc(-c3nc(C(C)C)n4c(C5=CCC(NC6COC6)CC5)cnc(N)c34)cc2F)cn1]

### Example 12

### N-(4-(8-Amino-3-isopropyl-5-(4-(oxetan-3-ylamino)cyclohex-1-en-1-yl) imidazo[1,5-a]pyrazin-1-yl)-2-fluorophenyl)-6-(trifluoromethyl)pyridine-3-sulfonamide

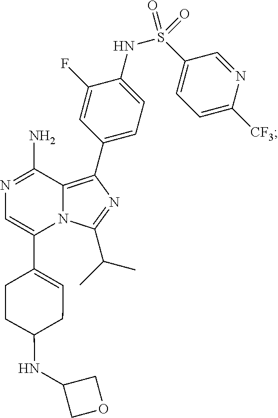 [SMILES: CC(C)c1nc(-c2ccc(NS(=O)(=O)c3ccc(C(F)(F)F)nc3)c(F)c2)c2c(N)ncc(C3=CCC(NC4COC4)CC3)n12]

### Example 13

### N-(4-(8-Amino-3-isopropyl-5-(4-(oxetan-3-ylamino)cyclohex-1-en-1-yl) imidazo[1,5-a]pyrazin-1-yl)-2-fluorophenyl)-4-methylpridine-2-sulfonamide

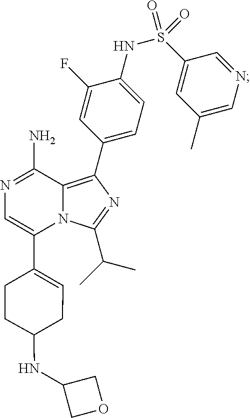 [SMILES: Cc1cncc(S(=O)(=O)Nc2ccc(-c3nc(C(C)C)n4c(C5=CCC(NC6COC6)CC5)cnc(N)c34)cc2F)c1]

### Example 14

### N-(4-(8-Amino-3-isopropyl-5-(4-(oxetan-3-ylamino)cyclohex-1-en-1-yl) imidazo[1,5-a]pyrazin-1-yl)-2-fluorophenyl)pyridine-2-sulfonamide

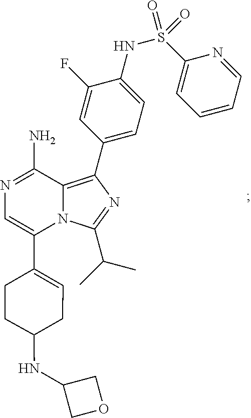 [SMILES: CC(C)c1nc(-c2ccc(NS(=O)(=O)c3ccccn3)c(F)c2)c2c(N)ncc(C3=CCC(NC4COC4)CC3)n12]

### Example 15

### N-(4-(8-Amino-3-isopropyl-5-(4-(oxetan-3-ylamino)cyclohex-1-en-1-yl) imidazo[1,5-a]pyrazin-1-yl)-2-fluorophenyl)-2-methylthiazole-4-sulfonamide

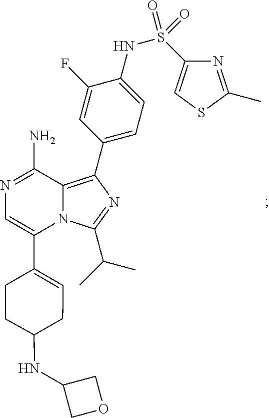 [SMILES: Cc1nc(S(=O)(=O)Nc2ccc(-c3nc(C(C)C)n4c(C5=CCC(NC6COC6)CC5)cnc(N)c34)cc2F)cs1]

### Example 16

### N-(4-(8-Amino-3-isopropyl-5-(4-((2-(methylsulfonyl)ethyl)amino)cyclohex-1-en-1-yl)imidazo[1,5-a]pyrazin-1-yl)-2-fluorophenyl)-2-chlorobenzenesulfonamide

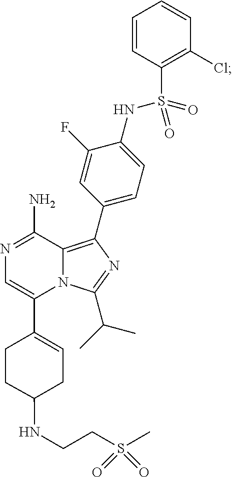 [SMILES: CC(C)c1nc(-c2ccc(NS(=O)(=O)c3ccccc3Cl)c(F)c2)c2c(N)ncc(C3=CCC(NCCS(C)(=O)=O)CC3)n12]

### Example 17

### N-(4-(8-Amino-5-(4-aminocyclohex-1-en-1-yl)-3-isopropylimidazo[1,5-a]pyrazin-1-yl)-2-fluorophenyl)-1-(2-methylthiazol-4-yl)methanesulfonamide

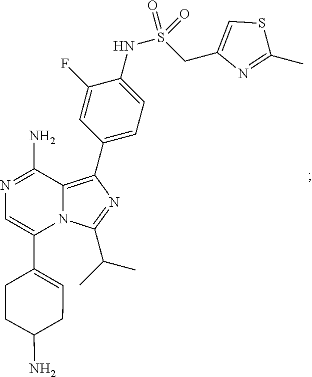 [SMILES: Cc1nc(CS(=O)(=O)Nc2ccc(-c3nc(C(C)C)n4c(C5=CCC(N)CC5)cnc(N)c34)cc2F)cs1]

### Example 18

### N-(4-(8-Amino-3-isopropyl-5-(4-morpholinocyclohex-1-en-1-yl)imidazo[1,5-a]pyrazin-1-yl)-2-fluorophenyl)-2-chlorobenzenesulfonamide

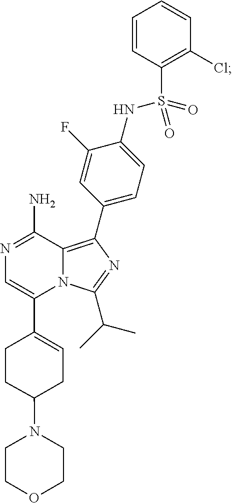 [SMILES: CC(C)c1nc(-c2ccc(NS(=O)(=O)c3ccccc3Cl)c(F)c2)c2c(N)ncc(C3=CCC(N4CCOCC4)CC3)n12]

### Example 19

### N-(4-(8-amino-3-isopropyl-5-(4-((2-methoxyethyl)amino)cyclohex-1-en-1-yl)imidazo[1,5-a]pyrazin-1-yl)-2-fluorophenyl)-2-chlorobenzenesulfonamide

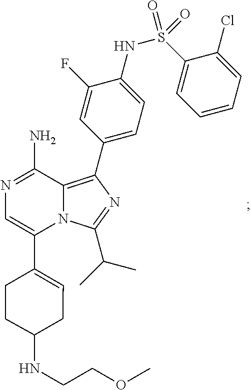 [SMILES: COCCNC1CC=C(c2cnc(N)c3c(-c4ccc(NS(=O)(=O)c5ccccc5Cl)c(F)c4)nc(C(C)C)n23)CC1]

### Example 20

### N-(5-(8-amino-3-isopropyl-5-(4-(oxetan-3-ylamino)cyclohex-1-en-1-yl)imidazo[1,5-a]pyrazin-1-yl)-6-methoxypyridin-2-yl)-2-chlorobenzenesulfonamide

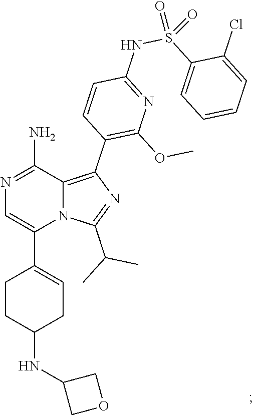 [SMILES: COc1nc(NS(=O)(=O)c2ccccc2Cl)ccc1-c1nc(C(C)C)n2c(C3=CCC(NC4COC4)CC3)cnc(N)c12]

### Example 21

### N-(5-(8-amino-3-isopropyl-5-(4-(oxetan-3-ylamino)cyclohex-1-en-1-yl)imidazo[1,5-a]pyrazin-1-yl)-6-methylpyridin-2-yl)-2-chlorobenzenesulfonamide

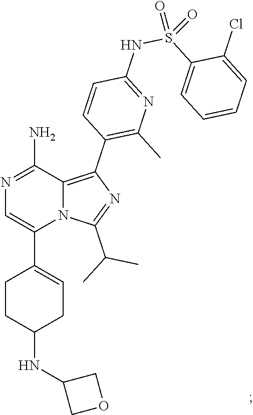 [SMILES: Cc1nc(NS(=O)(=O)c2ccccc2Cl)ccc1-c1nc(C(C)C)n2c(C3=CCC(NC4COC4)CC3)cnc(N)c12]

### Example 22

### N-(4-(8-amino-5-(4-dimethylamino)cyclohex-1-en-1-yl)-3-isopropylimidazo[1,5-a]pyrazin-1-yl)-2-fluorophenyl)-1-(2-chlorophenyl)methanesulfonamide

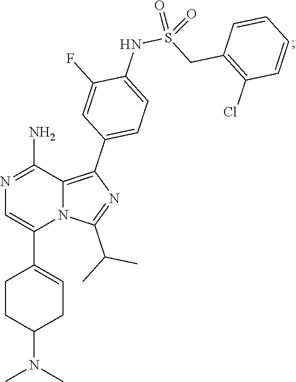 [SMILES: CC(C)c1nc(-c2ccc(NS(=O)(=O)Cc3ccccc3Cl)c(F)c2)c2c(N)ncc(C3=CCC(N(C)C)CC3)n12]

### Example 23

### N-(5-(8-amino-3-isopropyl-5-(4-(oxetan-3-ylamino)cyclohex-1-en-1-yl)imidazo[1,5-a]pyrazin-1-yl)-3-fluoropyridin-2-yl)-2-fluorobenzenesulfonamide

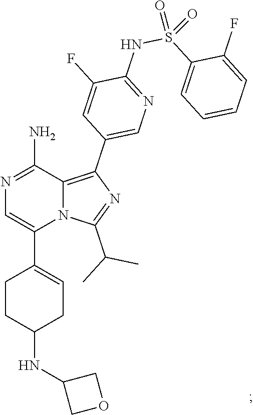 [SMILES: CC(C)c1nc(-c2cnc(NS(=O)(=O)c3ccccc3F)c(F)c2)c2c(N)ncc(C3=CCC(NC4COC4)CC3)n12]

### Example 24

### N-(4-(8-amino-3-isopropyl-5-(4-(oxetan-3-ylamino)cyclohex-1-en-1-yl)imidazo[1,5-a]pyrazin-1-yl)-2-fluorophenyl)-5-chloropyridine-2-sulfonamide

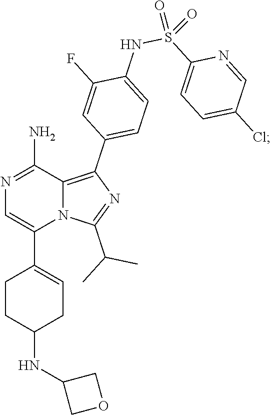 [SMILES: CC(C)c1nc(-c2ccc(NS(=O)(=O)c3ccc(Cl)cn3)c(F)c2)c2c(N)ncc(C3=CCC(NC4COC4)CC3)n12]

### Example 25

### N-(4-(8-amino-3-isopropyl-5-(4-(oxetan-3-ylamino)cyclohex-1-en-1-yl)imidazo[1,5-a]pyrazin-1-yl)-2-fluorophenyl)-2-methyloxazole-5-sulfonamide

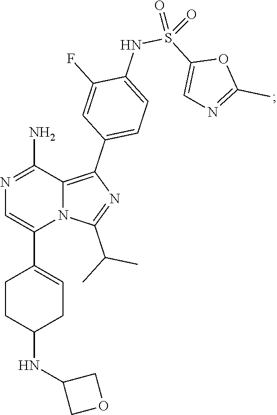 [SMILES: Cc1ncc(S(=O)(=O)Nc2ccc(-c3nc(C(C)C)n4c(C5=CCC(NC6COC6)CC5)cnc(N)c34)cc2F)o1]

or a salt, solvate, enantiomer, diastereoisomer, isotopologue or tautomer thereof.

In certain embodiments, the compound is:
(R)-8-Amino-1-(4-(((2-chlorophenyl)methyl)sulfonamido)-3-fluorophenyl)-3-isopropyl-N-(pyrrolidin-3-yl)imidazo[1,5-a]pyrazine-5-carboxamide;
(S)-8-Amino-1-(4-(((2-chlorophenyl)methyl)sulfonamido)-3-fluorophenyl)-3-isopropyl-N-(pyrrolidin-3-yl)imidazo[1,5-a]pyrazine-5-carboxamide;
(R)—N-(4-(8-Amino-3-isopropyl-5-(4-(oxetan-3-ylamino)cyclohex-1-en-1-yl)imidazo[1,5-a]pyrazin-1-yl)-2-fluorophenyl)-2-chlorobenzenesulfonamide;
(S)—N-(4-(8-Amino-3-isopropyl-5-(4-(oxetan-3-ylamino)cyclohex-1-en-1-yl)imidazo[1,5-a]pyrazin-1-yl)-2-fluorophenyl)-2-chlorobenzenesulfonamide;
(R)—N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-fluorobenzenesulfonamide;
(S)—N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-fluorobenzenesulfonamide;
(R)—N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-chlorobenzenesulfonamide;
(S)—N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-chlorobenzenesulfonamide;
(R)—N-(4-(8-Amino-3-isopropyl-5-(4-(oxetan-3-ylamino)cyclohex-1-en-1-yl) imidazo[1,5-a]pyrazin-1-yl)-3-chloro-2-fluorophenyl)-2-fluorobenzenesulfonamide;
(S)—N-(4-(8-Amino-3-isopropyl-5-(4-(oxetan-3-ylamino)cyclohex-1-en-1-yl) imidazo[1,5-a]pyrazin-1-yl)-3-chloro-2-fluorophenyl)-2-fluorobenzenesulfonamide;
(R)—N-(5-(8-Amino-3-isopropyl-5-(4-(oxetan-3-ylamino)cyclohex-1-en-1-yl) imidazo[1,5-a]pyrazin-1-yl)-3-fluoropyridin-2-yl)-2-chlorobenzenesulfonamide;
(S)—N-(5-(8-Amino-3-isopropyl-5-(4-(oxetan-3-ylamino)cyclohex-1-en-1-yl) imidazo[1,5-a]pyrazin-1-yl)-3-fluoropyridin-2-yl)-2-chlorobenzenesulfonamide;
(R)—N-(4-(8-Amino-3-isopropyl-5-(4-(oxetan-3-ylamino)cyclohex-1-en-1-yl) imidazo[1,5-a]pyrazin-1-yl)-2-fluorophenyl)-6-methoxypyridine-3-sulfonamide;
(S)—N-(4-(8-Amino-3-isopropyl-5-(4-(oxetan-3-ylamino)cyclohex-1-en-1-yl) imidazo[1,5-a]pyrazin-1-yl)-2-fluorophenyl)-6-methoxypyridine-3-sulfonamide;
(R)—N-(4-(8-Amino-3-isopropyl-5-(4-(oxetan-3-ylamino)cyclohex-1-en-1-yl) imidazo[1,5-a]pyrazin-1-yl)-2-fluorophenyl)-6-(trifluoromethyl)pyridine-3-sulfonamide;
(S)—N-(4-(8-Amino-3-isopropyl-5-(4-(oxetan-3-ylamino)cyclohex-1-en-1-yl) imidazo[1,5-a]pyrazin-1-yl)-2-fluorophenyl)-6-(trifluoromethyl)pyridine-3-sulfonamide;
(R)—N-(4-(8-Amino-3-isopropyl-5-(4-(oxetan-3-ylamino)cyclohex-1-en-1-yl) imidazo[1,5-a]pyrazin-1-yl)-2-fluorophenyl)-4-methylpyridine-2-sulfonamide;
(S)—N-(4-(8-Amino-3-isopropyl-5-(4-(oxetan-3-ylamino)cyclohex-1-en-1-yl) imidazo[1,5-a]pyrazin-1-yl)-2-fluorophenyl)-4-methylpyridine-2-sulfonamide;
(R)—N-(4-(8-Amino-3-isopropyl-5-(4-(oxetan-3-ylamino)cyclohex-1-en-1-yl) imidazo[1,5-a]pyrazin-1-yl)-2-fluorophenyl)pyridine-2-sulfonamide;
(S)—N-(4-(8-Amino-3-isopropyl-5-(4-(oxetan-3-ylamino)cyclohex-1-en-1-yl) imidazo[1,5-a]pyrazin-1-yl)-2-fluorophenyl)pyridine-2-sulfonamide;
(R)—N-(4-(8-Amino-3-isopropyl-5-(4-(oxetan-3-ylamino)cyclohex-1-en-1-yl) imidazo[1,5-a]pyrazin-1-yl)-2-fluorophenyl)-2-methylthiazole-4-sulfonamide;
(S)—N-(4-(8-Amino-3-isopropyl-5-(4-(oxetan-3-ylamino)cyclohex-1-en-1-yl) imidazo[1,5-a]pyrazin-1-yl)-2-fluorophenyl)-2-methylthiazole-4-sulfonamide;
(R)—N-(4-(8-Amino-3-isopropyl-5-(4-((2-(methylsulfonyl)ethyl)amino)cyclohex-1-en-1-yl)imidazo[1,5-a]pyrazin-1-yl)-2-fluorophenyl)-2-chlorobenzenesulfonamide
(S)—N-(4-(8-Amino-3-isopropyl-5-(4-((2-(methylsulfonyl)ethyl)amino)cyclohex-1-en-1-yl)imidazo[1,5-a]pyrazin-1-yl)-2-fluorophenyl)-2-chlorobenzenesulfonamide;
(R)—N-(4-(8-Amino-5-(4-aminocyclohex-1-en-1-yl)-3-isopropylimidazo[1,5-a]pyrazin-1-yl)-2-fluorophenyl)-1-(2-methylthiazol-4-yl)methanesulfonamide;
(S)—N-(4-(8-Amino-5-(4-aminocyclohex-1-en-1-yl)-3-isopropylimidazo[1,5-a]pyrazin-1-yl)-2-fluorophenyl)-1-(2-methylthiazol-4-yl)methanesulfonamide;
(R)—N-(4-(8-Amino-3-isopropyl-5-(4-morpholinocyclohex-1-en-1-yl)imidazo[1,5-a]pyrazin-1-yl)-2-fluorophenyl)-2-chlorobenzenesulfonamide;
(S)—N-(4-(8-Amino-3-isopropyl-5-(4-morpholinocyclohex-1-en-1-yl)imidazo[1,5-a]pyrazin-1-yl)-2-fluorophenyl)-2-chlorobenzenesulfonamide;
(R)—N-(4-(8-amino-3-isopropyl-5-(4-((2-methoxyethyl)amino)cyclohex-1-en-1-yl)imidazo[1,5-a]pyrazin-1-yl)-2-fluorophenyl)-2-chlorobenzenesulfonamide;
(S)—N-(4-(8-amino-3-isopropyl-5-(4-((2-methoxyethyl)amino)cyclohex-1-en-1-yl)imidazo[1,5-a]pyrazin-1-yl)-2-fluorophenyl)-2-chlorobenzenesulfonamide;
(R)—N-(5-(8-amino-3-isopropyl-5-(4-(oxetan-3-ylamino)cyclohex-1-en-1-yl)imidazo[1,5-a]pyrazin-1-yl)-6-methoxypyridin-2-yl)-2-chlorobenzenesulfonamide;
(S)—N-(5-(8-amino-3-isopropyl-5-(4-(oxetan-3-ylamino)cyclohex-1-en-1-yl)imidazo[1,5-a]pyrazin-1-yl)-6-methoxypyridin-2-yl)-2-chlorobenzenesulfonamide;
(R)—N-(5-(8-amino-3-isopropyl-5-(4-(oxetan-3-ylamino)cyclohex-1-en-1-yl)imidazo[1,5-a]pyrazin-1-yl)-6-methylpyridin-2-yl)-2-chlorobenzenesulfonamide;
(S)—N-(5-(8-amino-3-isopropyl-5-(4-(oxetan-3-ylamino)cyclohex-1-en-1-yl)imidazo[1,5-a]pyrazin-1-yl)-6-methylpyridin-2-yl)-2-chlorobenzenesulfonamide;
(R)—N-(4-(8-amino-5-(4-dimethylamino)cyclohex-1-en-1-yl)-3-isopropylimidazo[1,5-a]pyrazin-1-yl)-2-fluorophenyl)-1-(2-chlorophenyl)methanesulfonamide;
(S)—N-(4-(8-amino-5-(4-dimethylamino)cyclohex-1-en-1-yl)-3-isopropylimidazo[1,5-a]pyrazin-1-yl)-2-fluorophenyl)-1-(2-chlorophenyl)methanesulfonamide;
(R)—N-(5-(8-amino-3-isopropyl-5-(4-(oxetan-3-ylamino)cyclohex-1-en-1-yl)imidazo[1,5-a]pyrazin-1-yl)-3-fluoropyridin-2-yl)-2-fluorobenzenesulfonamide;
(S)—N-(5-(8-amino-3-isopropyl-5-(4-(oxetan-3-ylamino)cyclohex-1-en-1-yl)imidazo[1,5-a]pyrazin-1-yl)-3-fluoropyridin-2-yl)-2-fluorobenzenesulfonamide;
(R)—N-(4-(8-amino-3-isopropyl-5-(4-(oxetan-3-ylamino)cyclohex-1-en-1-yl)imidazo[1,5-a]pyrazin-1-yl)-2-fluorophenyl)-5-chloropyridine-2-sulfonamide;
(S)—N-(4-(8-amino-3-isopropyl-5-(4-(oxetan-3-ylamino)cyclohex-1-en-1-yl)imidazo[1,5-a]pyrazin-1-yl)-2-fluorophenyl)-5-chloropyridine-2-sulfonamide;
(R)—N-(4-(8-amino-3-isopropyl-5-(4-(oxetan-3-ylamino)cyclohex-1-en-1-yl)imidazo[1,5-a]pyrazin-1-yl)-2-fluorophenyl)-2-methyloxazole-5-sulfonamide;
(S)—N-(4-(8-amino-3-isopropyl-5-(4-(oxetan-3-ylamino)cyclohex-1-en-1-yl)imidazo[1,5-a]pyrazin-1-yl)-2-fluorophenyl)-2-methyloxazole-5-sulfonamide;
   or a salt, solvate, enantiomer, diastereoisomer, isotopologue, or tautomer thereof

In certain embodiments, the compound is an inhibitor of IRE1. In other embodiments, the compound is an inhibitor of IRE1α. In yet other embodiments, the compound is an inhibitor of IRE1α kinase activity. In yet other embodiments, the compound is an inhibitor of IRE1α RNase activity. In yet other embodiments, the compound binds the ATP binding site of IRE1α. In yet other embodiments, the compound binds IRE1α in the DFG-out conformation. In yet other embodiments, the compound binds IRE1α in the DFG-in conformation. In yet other embodiments, the compound induces the DFG-out conformation of IRE1α. In yet other embodiments, the compound is an inhibitor of IRE1α oligomerization. In yet other embodiments, the compound is an inhibitor of IRE1α dimerization. In yet other embodiments, the compound is an inhibitor of IRE1α phosphorylation. In yet other embodiments, the compound is an inhibitor of IRE1α autophosphorylation. In yet other embodiments, the compound is an inhibitor of apoptosis. In yet other embodiments, the compound is an inhibitor of IRE1α induced apoptosis. In yet other embodiments, the compound is an inhibitor of cell death. In yet other embodiments, the compound is an inhibitor of IRE1α induced cell death. In yet other embodiments, the compound is an inhibitor of a pathway induced by IRE1α phosphorylation. In yet other embodiments, the compound is an inhibitor of a pathway induced by IRE1α kinase activity. In yet other embodiments, the compound is an inhibitor of a pathway induced by IRE1α RNase activity. In yet other embodiments, the compound is an inhibitor of neuronal cell death. In yet other embodiments, the compound is a cytotoxic agent. In yet other embodiments, the compound is an anticancer agent. In yet other embodiments, the compound is an inhibitor of demyelination. In yet other embodiments, the compound is an antidiabetic agent. In yet other embodiments, the compound is a neuroprotective agent. In yet other embodiments, the compound protects against loss of photoreceptor cells. In yet other embodiments, the compound is an inhibitor of fibrosis. In yet other embodiments, the compound decreases apoptosis in cells under ER stress. In yet other embodiments, the compound decreases apoptosis in cells under ER stress, but not cells that are under the same conditions but not under ER stress. In yet other embodiments, the compound decreases apoptosis in cells under ER stress more than in cells that are under the same conditions but not under ER stress. In yet other embodiments, the compound decreases cleavage of miR-17. In yet other embodiments, the compound decreases IRE1α associated cleavage of miR-17. In yet other embodiments, the compound decreases cleavage of miR-34a. In yet other embodiments, the compound decreases IRE1α associated cleavage of miR-34a. In yet other embodiments, the compound decreases cleavage of miR-96. In yet other embodiments, the compound decreases IRE1α associated cleavage of miR-96. In yet other embodiments, the compound decreases cleavage of miR-125b. In yet other embodiments, the compound decreases IRE1α associated cleavage of miR-125b. In yet other embodiments, the compound decreases XBP 1 mRNA splicing. In yet other embodiments, the compound decreases IRE1α associated XBP1 mRNA splicing. In yet other embodiments, the compound decreases UPR signaling. In yet other embodiments, the compound decreases IRE1α associated UPR signaling. In yet other embodiments, the compound decreases terminal UPR signaling. In other embodiments, the compound decreases IRE1α associated terminal UPR signaling.

The compounds described herein may form salts with acids and/or bases, and such salts are included in the present invention. In certain other embodiments, the salts are pharmaceutically acceptable salts. The term “salts” embraces addition salts of free acids and/or bases that are useful within the methods of the invention. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hemisulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate).

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, ammonium, N,N′-dibenzylethylene-diamine, chloroprocaine, choline, diethanolamine, ethylenediamine, meglumine (N-methylglucamine) and procaine.

All of these salts may be prepared from the corresponding compound by reacting, for example, the appropriate acid or base with the compound. Salts may be comprised of a fraction of less than one, one, or more than one molar equivalent of acid or base with respect to any compound of the invention.

In certain other embodiments, the at least one compound of the invention is a component of a pharmaceutical composition further including at least one pharmaceutically acceptable carrier.

### Compound Preparation:

The compounds of this invention can be made by a variety of methods, including well-known standard synthetic methods. Illustrative general synthetic methods are set out below and then specific compounds of the invention are prepared in the working examples. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. In all of the schemes described below, protecting groups for sensitive or reactive groups are employed where necessary in accordance with general principles of synthetic chemistry. Protecting groups are manipulated according to standard methods of organic synthesis (T.W. Green and P.G.M. Wuts, (1991) Protecting Groups in Organic Synthesis, John Wiley & Sons, incorporated by reference with regard to protecting groups).

In the procedures that follow, some of the starting materials are identified through a “Step” or “Example” number. This is provided merely for assistance to the skilled chemist. The starting material may not necessarily have been prepared from the batch referred to.

When reference is made to the use of a “similar” or “analogous” procedure, as will be appreciated by those skilled in the art, such a procedure may involve minor variations, for example reaction temperature, reagent/solvent amount, reaction time, work-up conditions or chromatographic purification conditions.

The synthesis of the compounds of the general formula (I), and pharmaceutically acceptable derivatives and salts thereof, can be accomplished as outlined below, for example in Schemes 1-5, by those skilled in the art. Starting materials are commercially available or are made from commercially available starting materials using methods known to those skilled in the art.

The compounds of the invention may possess one or more stereocenters, and each stereocenter may exist independently in either the (R) or (S) configuration. In certain other embodiments, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain other embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain other embodiments, the compounds described herein exist in solvated forms with pharmaceutically acceptable solvents such as water, and ethanol. In other embodiments, the compounds described herein exist in unsolvated form.

In certain other embodiments, the compounds of the invention exist as tautomers. All tautomers are included within the scope of the compounds recited herein.

In certain other embodiments, compounds described herein are prepared as prodrugs. A “prodrug” is an agent converted into the parent drug in vivo. In certain other embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain other embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain other embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain other embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain other embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and in the art. General methods for the preparation of compound as described herein are modified by the use of appropriate reagents and conditions, for the introduction of the various moieties found in the formula as provided herein.

### Methods

The invention includes methods of treating disorders associated with ER stress. In certain embodiments, the invention provides methods of treating a disease or disorder in a subject, the method comprising administering to the subject a therapeutically effective amount of one or more compounds of the invention, or pharmaceutically acceptable salts, solvates, enantiomers, diastereoisomers, or tautomers thereof. In other embodiments, the subject is in need of the treatment.

In certain embodiments, the disease or disorder is selected from the group consisting of a neurodegenerative disease, a demyelinating disease, cancer, an eye disease, a fibrotic disease, and diabetes.

In certain embodiments, the disease is a neurodegenerative disease selected from the group consisting of retinitis pigmentosa, amyotrophic lateral sclerosis, retinal degeneration, macular degeneration, Parkinson's Disease, Alzheimer's Disease, Huntington's Disease, Prion Disease, Creutzfeldt-Jakob Disease, and Kuru.

In certain embodiments, the disease is a demyelinating disease selected from the group consisting of Wolfram Syndrome, Pelizaeus-Merzbacher Disease, Transverse Myelitis, Charcot-Marie-Tooth Disease, and Multiple Sclerosis.

In certain embodiments, the disease is cancer. In other embodiments, the disease is multiple myeloma.

In certain embodiments, the disease is diabetes. In other embodiments, the disease is selected from the group consisting of type I diabetes and type II diabetes.

In certain embodiments, the disease is an eye disease selected from the group consisting of retinitis pigmentosa, retinal degeneration, macular degeneration, and Wolfram Syndrome.

In certain embodiments, the disease is a fibrotic disease selected from the group consisting of idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), and hepatic fibrosis.

Without being limited to any single theory, the compounds of the invention treat the aforementioned diseases and disorders by modulating the activity of an IRE1 protein. In certain embodiments, the compounds inhibit the activity of an IRE1 protein.

In certain embodiments, the compounds of the invention modulate kinase activity of an IRE1 protein. In other embodiments, the compounds of the invention modulate autophosphorylation activity of an IRE1 protein. In yet other embodiments, the compounds of the invention modulate oligomerization activity of an IRE1 protein. In yet other embodiments, the compounds of the invention modulate dimerization activity of an IRE1 protein.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. The pharmaceutical compositions useful for practicing the invention may be administered to deliver a dose of from 1 ng/kg/day and 100 mg/kg/day. One of ordinary skill in the art would be able to study the relevant factors and make the determination regarding the effective amount of the therapeutic compound without undue experimentation.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

In certain other embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier. In yet other embodiments, the compound of the invention is the only biologically active agent (i.e., capable of treating or preventing diseases and disorders related to IRE1) in the composition. In yet other embodiments, the compound of the invention is the only biologically active agent (i.e., capable of treating or preventing diseases and disorders related to IRE1) in therapeutically effective amounts in the composition.

In certain other embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 g to about 9,500 mg, about 40 g to about 9,000 mg, about 75 g to about 8,500 mg, about 150 g to about 7,500 mg, about 200 g to about 7,000 mg, about 300 g to about 6,000 mg, about 500 g to about 5,000 mg, about 750 g to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain other embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include intravitreal, oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravitreal, intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intravitreal, intraperitoneal, intramuscular, intrasternal injection, and kidney dialytic infusion techniques.

### Intravitreal Administration

As used herein, “intravitreal administration” of a pharmaceutical composition includes administration into the vitreous fluid within the eye of a subject. Intravitreal administration includes, but is not limited to, administration of a pharmaceutical composition into the eye of a subject by injection of the composition. In some embodiments, the pharmaceutical composition can be administered through the use of a hypodermic needle or through a surgical incision. Preferably, administration takes place through the sclera of the eye, avoiding damage to the cornea or lens.

In certain embodiments, the pharmaceutical composition of the invention can be formulated for administration to the eye of the subject with sustained release over a period of 3-12 months.

### Controlled Release Formulations and Drug Delivery Systems

In certain other embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form. In certain embodiments, the compounds of the invention can be formulated for sustained release over a period of 3-12 months.

For sustained release, the compounds may be formulated with a suitable polymer or hydrophobic material that provides sustained release properties to the compounds. As such, the compounds useful within the methods of the invention may be administered in the form of microparticles, for example by injection, or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 5 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain other embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 5 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### General Experimental Details:

Reactions were not carried out under an inert atmosphere unless specified, and all solvents and commercial reagents were used as received.

Purification by chromatography refers to purification using the COMBIFLASH® Companion purification system or the Biotage SP1 purification system. Where products were purified using an Isolute® SPE Si II cartridge, ‘Isolute SPE Si cartridge’ refers to a pre-packed polypropylene column containing unbonded activated silica with irregular particles with average size of 50 μm and nominal 60 Å porosity. Fractions containing the required product (identified by TLC and/or LCMS analysis) were pooled and the solvent removed by evaporation to give the desired product. Where thin layer chromatography (TLC) has been used, it refers to silica-gel TLC using plates, typically 3×6 cm silica-gel on aluminum foil plates (e.g. Fluka 60778) with a fluorescent indicator (254 nm). Microwave experiments were carried out using a Biotage Initiator 60™ which uses a single-mode resonator and dynamic field tuning. Temperature from 40-250° C. can be achieved, and pressures of up to 30 bar can be reached.

NMR spectra were obtained on a Bruker Avance 400 MHz, 5 mm QNP probe H, C, F, P, single Z gradient, two channel instrument running TopSpin 2.1 or on a Bruker Avance III 400 MHz, 5 mm BBFO Plus probe, single Z gradient, two channel instrument running TopSpin 3.0.

Compound names were standardly generated using the Struct>Name function in ChemDraw Professional 15.1.

### Analytical LC-MS Conditions:

Method 1: Acquity UPLC with 996 DAD detector and Quattro Micro Mass Spectrometer. Column: Acquity UPLC CSH C18 (1.7 μm 50×2.1 mm), maintained at 40° C. Conditions: 0.1% aqueous formic acid [eluent A]; MeCN (containing 0.1% formic acid) [eluent B]. Gradient: 3% B for 0.15 min, then 3 to 99% B over 2.15 mins, then isocratic for 0.1 mins at 1 mL/min.

Method 2: Acquity H-Class UPLC with quaternary pump/PDA detector and QDa Mass Spectrometer. Column: Waters Acquity BEH UPLC column C18 (1.7 μm 50×2.1 mm), maintained at 40° C. Conditions: 7.66 mM aqueous ammonia [eluent A]; 7.66 mM ammonia in MeCN [eluent B]. Gradient: 3 to 97% B over 4 mins, then isocratic for 0.4 mins at 0.8 mL/min.

Method 3: Acquity H-Class UPLC with quaternary pump/PDA detector and QDa Mass Spectrometer. Column: Waters Acquity BEH UPLC column C18 (1.7 μm 50×2.1 mm), maintained at 40° C. Conditions: 7.66 mM aqueous ammonia [eluent A]; 7.66 mM ammonia in MeCN [eluent B]. Gradient: 3 to 97% B over 1.5 mins, then isocratic for 0.4 mins at 0.8 mL/min.

Method 4: Acquity H-Class UPLC with quaternary pump/PDA detector and QDa Mass Spectrometer. Column: Acquity UPLC CSH C18 (1.7 μm 50×2.1 mm), maintained at 40° C. Conditions: 0.1% aqueous formic acid [eluent A]; MeCN (containing 0.1% formic acid) [eluent B]. Gradient: 3 to 99% B over 1.5 min, then isocratic for 0.4 mins at 1 mL/min.

### QCLC-MS Conditions:

QC Method 1: Acquity UPLC (binary pump/PDA detector)+ZQ Mass Spectrometer. Column: reverse-phase column—Acquity UPLC BEH C18 (1.7 μm, 100×2.1 mm), maintained at 40° C. Conditions: 0.1% aqueous formic acid [eluent A]; MeCN (containing 0.1% formic acid) [eluent B]. Gradient: isocratic at 5% B for 0.4 min then 5 to 95% B over 5.6 min at 0.4 mL/min.

QC Method 2: Acquity i-Class (quaternary pump/PDA detector)+Quattro Micro Mass Spectrometer. Column: reverse-phase column—Acquity UPLC BEH C18 1.7 μm, 100×2.1 mm) maintained at 40° C., elution with A: water+0.1% formic acid; B: MeCN+0.1% formic acid. Gradient: isocratic at 5% B for 0.4 min then 5 to 95% B over 5.6 min at 0.4 mL/min.

QC Method 3: Waters SQD2, single quadrapole UPLC-MS. Column: Acquity UPLC BEH Shield RP18 (1.7 μm, 100×2.1 mm). Conditions: 10 mM aqueous ammonium bicarbonate [eluent A]; MeCN [eluent B]. Gradient: 5 to 100% B over 4.9 min at 0.5 m/min.

### SFC and HPLC Methods:

Preparative SFC: Waters Thar Prep100 preparative SFC system (P200 CO₂ pump, 2545 modifier pump, 2998 UV/VIS detector, 2767 liquid handler with Stacked Injection Module). Column: Phenomenex Lux Cellulose-4 or YMC Cellulose-SC (5 μm, 10-21.2×250 mm), maintained at 40° C. Conditions: supercritical fluid CO₂ and eluents chosen from MeOH, EtOH, IPA, MeCN, EtOAc, THF with modifiers chosen from Et₂NH or formic acid as specified. Gradient/isocratic as specified at 100 mL/min, 120 bar (or as appropriate).

Analytical SFC was carried out on a similar system using smaller columns and lower flow rates.

### Preparative HPLC:

Gilson semi-prep system, Waters XSELECT CSH C18 RP (5 μM, 19×250 mm) column, using 5-60% MeCN in H₂O (+0.1% formic acid) at 18 mL/min over 10 minutes RAMP, UV detection at 220 nm.

### Synthesis

Examples 1-4 can be made according to the chemistry outlined in Scheme 1. Treatment of 4-bromo-2-fluoroaniline 1A with a sulfonyl chloride such as (2-chlorophenyl) sulfonyl chloride gives the sulfonamide intermediate 1B, which can be converted to the boronate 1C, and then undergoes Pd(0)-catalyzed coupling with the heteroaromatic bromide 1R to give a coupling product such as 1D. Acid deprotection affords the final product 1E and 1F after HPLC purification. Furthermore, additional compounds of the invention can be made by reduction of an imine formed from suitable ketones such as 1,3-oxetanone to give final products 1G and 1H after HPLC purification. Additional compounds can be formed by direct alkylation of 1E and 1F with a suitable alkylating reagents.

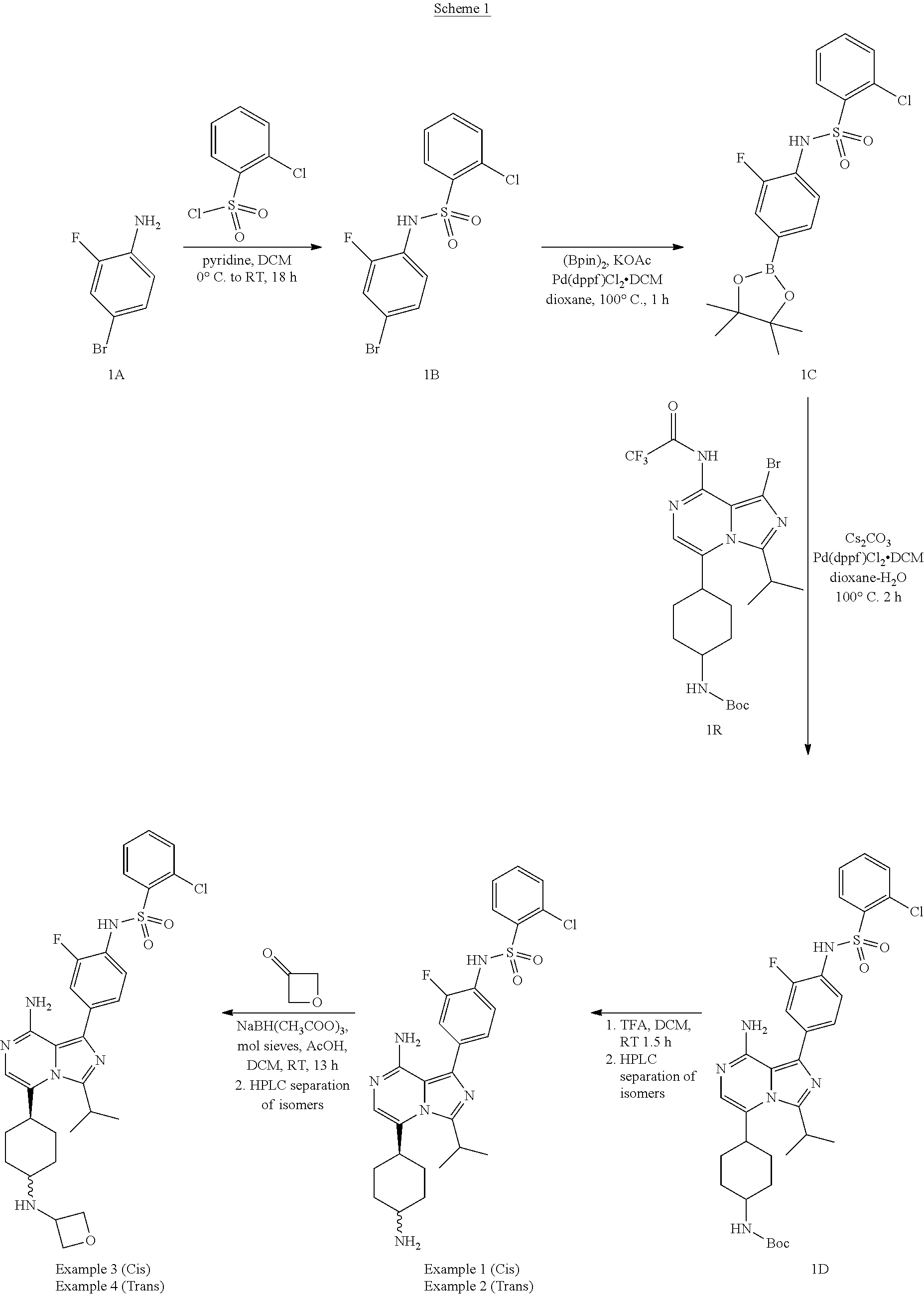

Intermediate 1R can be prepared according to the chemistry outlined in Scheme 2. Treatment of methyl 3-amino-6-bromopyrazine-2-carboxylate 1I with tert-butyl (4-(4,4,5,5-tetramethyl-1,3,2-dioxaborolan-2-yl)cyclohex-3-en-1-yl)carbamate under Suzuki coupling conditions gives the intermediate 1J, which can be reduced to the alcohol 1K using lithium aluminium hydride. Conversion of the alcohol 1K to the amine by a Staudinger reduction of the azide to give 1L and then amide bond formation gives intermediate 1M. The double bond in the cyclohexene ring of 1M can be reduced by Pd-catalyzed hydrogenation to give intermediate 1N which is protected by trifluoro-acetylation to give 1P. Cyclization to the imidazo[1,5-a]pyrazine ring system with Burgess reagent and then bromination with NBS gives the required intermediate 1R as mixture of cis/trans geometric isomers.

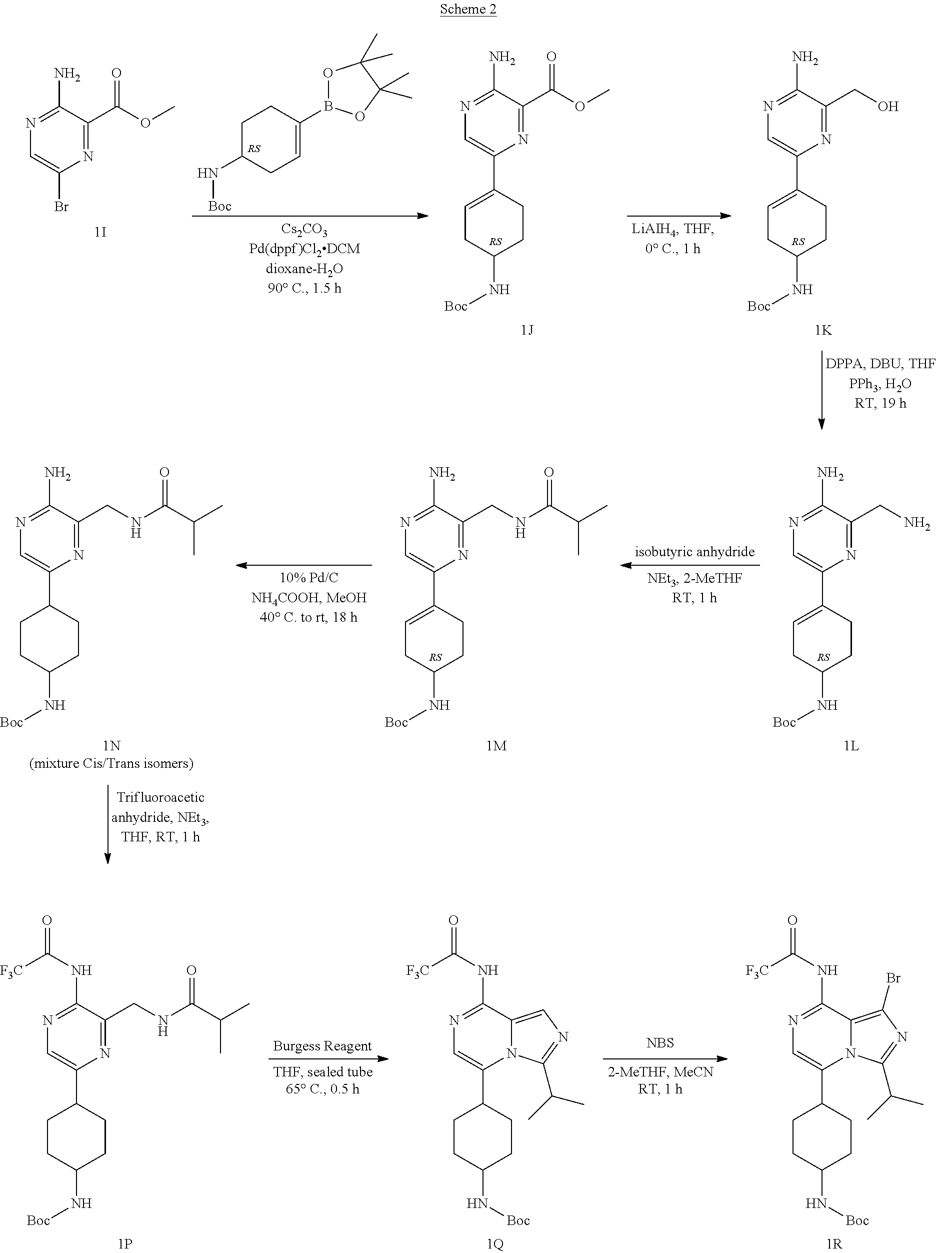

### Example 1: N-(4-(8-Amino-5-((1s,4s)-4-aminocyclohexyl)-3-isopropylimidazo[1,5-a]pyrazin-1-yl)-2-fluorophenyl)-2-chlorobenzenesulfonamide (Cis Isomer)

### Step 1: N-(4-Bromo-2-fluorophenyl)-2-chlorobenzenesulfonamide 1B

A solution of 4-bromo-2-fluoroaniline 1A (190.1 g, 1 mol) in DCM (2 L) was treated pyridine (242.6 mL, 3 mol) and cooled in an ice bath prior to addition of 2-chlorobenzene sulfonyl chloride (161.5 mL, 1.18 mol) over 10 mins to maintain the internal temperature 10° C. The ice bath was removed and the mixture stirred at RT for 18 h. The mixture was treated with 2M HCl (1 L) and the aqueous layer extracted with DCM. The combined organic layers were washed with saturated brine, dried (Na₂SO₄) and concentrated in vacuo. The residue was triturated with a solution of 10% DCM/Et₂O to give the title compound as an off-white solid (330.5 g, 91%). LCMS (Method 1): Rₜ=1.70 min, m/z=361.8 [M(³⁵Cl)−H].

### Step 2: 2-Chloro-N-(2-fluoro-4-(4,4,5,5-tetramethyl-1,3,2-dioxaborolan-2-yl)phenyl) benzenesulfonamide 1C

A mixture of N-(4-bromo-2-fluorophenyl)-2-chlorobenzenesulfonamide 1B (54.8 g, 150.3 mmol), bis(pinacolato)diboron (45.8 g, 180.4 mmol), PdCl₂(dppf).DCM (6.14 g, 7.51 mmol) and KOAc (36.88 g, 375.7 mmol) in 1,4-dioxane (600 mL) was purged with nitrogen then heated at 100° C. for 2 h. The mixture was cooled and diluted with water then extracted with EtOAc. The combined organic layer was washed with water, brine, dried (Na₂SO₄), filtered, and concentrated in vacuo. The resultant residue was purified through a pad of silica-gel, loading in DCM and eluting with 0-10% EtOAc in DCM. Product fractions were combined and triturated with Et₂O and collected by filtration to give the desired product as an off-white solid (34.1 g, 55%). The mother liquor was concentrated in vacuo then triturated with a 1:1 mixture of Et₂O/cyclohexane, which gave a second crop of the title compound (20.8 g, 34%). LCMS (Method 2.1): Rₜ=1.57 min, m/z=410.1 [M(³⁵Cl)−H].

### Step 3: tert-Butyl (4-(8-amino-1-(4-((2-chlorophenyl)sulfonamido)-3-fluorophenyl)-3-isopropylimidazo[1,5-a]pyrazin-5-yl)cyclohexyl)carbamate 1D

A mixture of tert-butyl (4-(1-bromo-3-isopropyl-8-(2,2,2-trifluoroacetamido)imidazo[1,5-a]pyrazin-5-yl)cyclohexyl)carbamate 1R (300 mg, 0.55 mmol), 2-chloro-N-(2-fluoro-4-(4,4,5,5-tetramethyl-1,3,2-dioxaborolan-2-yl)phenyl) benzenesulfonamide 1C (270 mg, 0.656 mmol), PdCl₂(dppf).DCM (22 mg, 0.027 mmol) and cesium carbonate (535 mg, 1.64 mmol) in a mixture of 1,4-dioxane (4.5 mL) and water (1.5 mL) was purged with nitrogen then heated at 100° C. for 2 h. The mixture was cooled and partitioned between water and 2-Me-THF and the aqueous layer was extracted with 2-Me-THF. The combined organic layers were washed with water and saturated brine, dried (Na₂SO₄) and concentrated in vacuo. The resultant residue was purified by silica-gel chromatography, eluting with 0-5% MeOH in DCM. The solvent was removed in vacuo and the residue purified by silica-gel chromatography, eluting with 0-30% acetone in DCM, to give the title compound as a mixture of cis and trans isomers as an off-white foam (311 mg, 86%). LCMS (Method 2): Rₜ=2.06 min and 2.08 min, m/z=657.3 [M(³⁵Cl)−H].

### Step 4: N-(4-(8-Amino-5-((1s,4s)-4-aminocyclohexyl)-3-isopropylimidazo[1,5-a]pyrazin-1-yl)-2-fluorophenyl)-2-chlorobenzenesulfonamide 1E (Cis Isomer)

Trifluoroacetic acid (533 μL, 6.96 mmol) was added to a solution of tert-butyl (4-(8-amino-1-(4-((2-chlorophenyl)sulfonamido)-3-fluorophenyl)-3-isopropylimidazo[1,5-a]pyrazin-5-yl)cyclohexyl)carbamate 1D (305 mg, 0.464 mmol) in dry DCM (1.5 mL) under argon at 0° C. and stirred for 1.5 h at RT. The mixture was concentrated in vacuo and evaporated with toluene. The residue was purified by C18 HPLC chromatography eluting with 20-55% acetonitrile in 0.10% NH₄OH_{aq}. Some of the material was isolated as a mixed fraction of isomers (71 mg, 27%, 2:1 ratio of 1E and 1F). The title compound eluted as the first compound from the chromatography step and was isolated as an off-white solid (39 mg, 15%). LCMS (QC Method 1): Rₜ=2.75 min, m/z=557.1 [M(³⁵Cl)−H]. ¹H NMR (400 MHz, d₆-DMSO) δ: 8.44 (2H, s), 7.99-7.93 (1H, m), 7.43-7.38 (1H, m), 7.36-7.29 (2H, m), 7.12 (1H, t, J=8.8 Hz), 7.02 (1H, dd, J=12.5 and 2.1 Hz), 6.86 (1H, dd, J=8.4 and 2 Hz), 6.83 (1H, s), 5.76 (2H, s), 3.46-3.35 (1H, m), 3.15-3.11 (1H, m) 1.90-1.71 (8H, m), 1.35 (6H, d, J=7.0 Hz).

### Example 2: N-(4-(8-Amino-5-((1r,4r)-4-aminocyclohexyl)-3-isopropylimidazo[1,5-a]pyrazin-1-yl)-2-fluorophenyl)-2-chlorobenzenesulfonamide, (Trans Isomer)

The title compound was obtained as the second eluting isomer from the chromatography in Step 4 and isolated as an off-white solid (8.8 mg, 4%). LCMS (QC Method 1): Rₜ=2.60 min, m/z=557.1 [M(³⁵Cl)−H]. ¹H NMR (400 MHz, d₆-DMSO) δ: 7.97-7.94 (1H, m), 7.42-7.39 (1H, m), 7.36-7.29 (2H, m), 7.12 (1H, t, J=8.8 Hz), 7.04-7.00 (1H, m), 6.85 (1H, dd, J=8.3 and 2.1 Hz), 6.76 (1H, d, J=5.8 Hz), 5.72 (2H, s), 3.21-3.11 (1H, m); 2.92-2.86 (1H, m), 1.98-1.85 (4H, m), 1.50-1.40 (2H, m), 1.36 (6H, d, J=6.4 Hz), 1.26-1.14 (2H, m).

### Example 3: N-(4-(8-Amino-3-isopropyl-5-((1s,4s)-4-(oxetan-3-ylamino)cyclohexyl)imidazo[1,5-a]pyrazin-1-yl)-2-fluorophenyl)-2-chlorobenzenesulfonamide, (Cis Isomer)

3-Oxetanone (17 μL, 0.26 mmol), acetic acid (50 μL), and 240 mg 4 Å molecular sieves were added to a 2:1 ratio mixture of 1E:1F isolated from Step 4 (120 mg, 1.04 mmol) dissolved in dry DCM (5 mL) and stirred at room temperature for 15 h. Sodium triacetoxyborohydride (137 mg, 0.6 mmol) was added and the mixture continued to stir for a further 1 h. The molecular sieves were removed by filtration and the filtrate diluted with EtOAc and washed with water, saturated brine, dried (Na₂SO₄), filtered and concentrated in vacuo. The residue was purified by silica-gel chromatography, eluting with 0-20% 2M NH₃/MeOH in DCM, giving the title compound as the first eluting isomer (39 mg, 29%). LCMS (QC Method 1): Rₜ=2.75 min, m/z=613.2 [M(³⁵Cl)−H]. ¹H NMR (400 MHz, CDCl₃) δ: 8.10-8.08 (1H, m), 7.61 (1H, t, J=8.1 Hz), 7.53-7.51 (2H, m), 7.40-7.35 (2H, m), 7.34-7.28 (1H m), 6.90 (1H s), 4.84 (2H, t, J=7.0 Hz), 4.46 (2H, t, J=6.4 Hz), 4.04 (1H, p, J=6.5 Hz), 3.36 (1H, p, J=7.1 Hz), 3.07-2.99 (1H, m), 2.96 (1H, t, J=3.05 Hz), 1.93-1.72 (6H, m), 1.70-1.60 (2H, m), 1.42 (6H, d, J=6.69 Hz).

### Example 4: N-(4-(8-Amino-3-isopropyl-5-((1r,4r)-4-(oxetan-3-ylamino)cyclohexyl)imidazo[1,5-a]pyrazin-1-yl)-2-fluorophenyl)-2-chlorobenzene sulfonamide, (Trans Isomer)

The title compound was obtained as the second eluting isomer from the chromatography step in the synthesis of Example 3 and was isolated as an off-white solid (24 mg, 18%). LCMS (QC Method 1) Rₜ=2.60 min, m/z=613.1 [M(³⁵Cl)−H]. ¹H NMR (400 MHz, CDCl₃) δ: 8.11-8.07 (1H, m), 7.61 (1H, t, J=8.2 Hz), 7.52-7.51 (2H, m), 7.41-7.32 (2H, m), 7.31-7.27 (1H, m), 6.83 (1H, s), 4.86 (2H, t, J=6.9 Hz), 4.45 (2H, t, J=6.5 Hz), 4.11 (1H, p, J=6.4 Hz), 3.34 (1H, p, J=6.8 Hz), 3.04-2.95 (1H, m), 2.64-2.55 (1H, m), 2.12-1.97 (5H, m), 1.57-1.45 (1H, m), 1.42 (6H, d, J=6.7 Hz), 1.34-1.22 (4H, m).

### Intermediate 1R

### Step 1. Methyl 3-amino-6-(4-((tert-butoxycarbonyl)amino)cyclohex-1-en-1-yl)pyrazine-2-carboxylate 1J

Cesium carbonate (25.3 g, 77.6 mmol) in water (54 mL) was added to a mixture of methyl 3-amino-6-bromopyrazine-2-carboxylate 1I (6.0 g, 26 mmol), tert-butyl (4-(4,4,5,5-tetramethyl-1,3,2-dioxaborolan-2-yl)cyclohex-3-en-1-yl)carbamate (10 g, 31 mmol), and PdCl₂(dppf).DCM (2.1 g, 2.6 mmol) in 1,4-dioxane (216 mL) and purged with argon then heated at 90° C. for 1.5 h. The 1,4-dioxane was removed in vacuo, the residue diluted with EtOAc and washed with water, saturated brine, dried (Na₂SO₄) and concentrated in vacuo. The residue was purified by silica-gel chromatography, eluting with 0-100% EtOAc in cyclohexane, giving the title compound as an off-white solid (3.17 g, 35%). LCMS (Method 3): Rₜ=1.38 min, m/z=347.2 [M−H].

### Step 2. tert-Butyl (4-(5-amino-6-(hydroxymethyl)pyrazin-2-yl)cyclohex-3-en-1-yl)carbamate 1K

Under an argon atmosphere 2M lithium aluminium hydride in THF (9.5 mL, 19.1 mmol) was added dropwise to a solution of methyl 3-amino-6-(4-((tert-butoxycarbonyl)amino)cyclohex-1-en-1-yl)pyrazine-2-carboxylate 1J (3.2 g, 9.1 mmol) in anhydrous THF (40 mL), maintaining the temperature at 0° C. Stirring was continued at 0° C. for 0.5 h, TBME was added (40 mL) and the reaction was quenched by dropwise addition of water (46 mL) then stirred for 5 minutes, addition of 1N NaOH (78 mL) with further stirring for 5 min and another addition of water (62 mL). The reaction was extracted with EtOAc and the combined organic extracts were washed with water, brine, dried (Na₂SO₄) and concentrated in vacuo. The residue was purified by silica-gel chromatography, eluting with 0-100% EtOAc in cyclohexane, to afford the title compound as a white solid (1.33 g, 46%). LCMS (Method 4): Rₜ=1.06 min, m/z=321.1 [M+H].

### Step 3. tert-Butyl (4-(5-amino-6-(aminomethyl)pyrazin-2-yl)cyclohex-3-en-1-yl)carbamate 1L

Under an atmosphere of argon, diphenylphosphoryl azide (5.2 mL, 23.97 mmol) was added dropwise to a solution of tert-butyl (4-(5-amino-6-(hydroxymethyl)pyrazin-2-yl)cyclohex-3-en-1-yl)carbamate 1K (3.84 g, 11.99 mmol) and 1,8-diazabicyclo[5.4.0]undec-7-ene (3.6 mL, 23.97 mmol) in THF (220 mL) stirring at RT. Stirring was continued for a further 2 h then the solution was diluted with water (22 mL), triphenylphosphine (6.3 g, 23.97 mmol) was added and stirring continued for a further 18 h at RT. The reaction was concentrated in vacuo to remove the THF, diluted with EtOAc, washed with water, saturated brine, dried (Na₂SO₄) and concentrated in vacuo. The residue was purified by silica-gel chromatography, eluting with 0-10% NH₃/MeOH in DCM, to afford the title compound as an off-white solid (1.43 g, 37%). LCMS (Method 4): Rₜ=0.72 min, m/z=320.1 [M+H].

### Step 4. tert-Butyl (4-(5-amino-6-(isobutyramidomethyl)pyrazin-2-yl)cyclohex-3-en-1-yl)carbamate 1M

Isobutyric anhydride (0.73 mL, 4.38 mmol) was added to a solution of tert-butyl (4-(5-amino-6-(aminomethyl)pyrazin-2-yl)cyclohex-3-en-1-yl)carbamate 1L (1.4 g, 4.38 mmol) and triethylamine (1.2 mL, 8.77 mmol) in 2-Me-THF (25 mL). Stirring was continued for 1 h at RT and then the reaction was diluted with 2-Me-THF, washed with 10% citric acid in water, water, saturated brine, dried (Na₂SO₄) and concentrated in vacuo. The title compound was isolated by trituration of the residue with Et₂O and filtration of the resulting cream solid (1.65 g, 96%). LCMS (Method 1): Rₜ=1.34 min, m/z=390.2 [M+H].

### Step 5. tert-Butyl (4-(5-amino-6-(isobutyramidomethyl)pyrazin-2-yl)cyclohexyl)carbamate 1N

10% Pd/C (652 mg, 0.614 mmol) was added to a solution of tert-butyl (4-(5-amino-6-(isobutyramidomethyl)pyrazin-2-yl)cyclohex-3-en-1-yl)carbamate 1M (2.39 g, 6.14 mmol) and ammonium formate (5.8 g, 92 mmol) in MeOH (67 mL) at RT. The reaction was heated to 50° C. and stirred for 45 min then the heat removed and stirring continued for 15 min. The reaction was filtered through a pad of celite flushing with MeOH. The eluent was concentrated in vacuo, diluted with EtOAc and washed with water, saturated brine, dried (Na₂SO₄) and concentrated in vacuo. The resultant residue was purified by silica-gel chromatography, eluting with 0-10% MeOH in DCM, to afford the title compound as a white foam (1.68 g, 69%). LCMS (Method 4): Rₜ=1.1 min, m/z=392.2 [M+H].

### Step 6. tert-Butyl (4-(6-(isobutyramidomethyl)-5-(2,2,2-trifluoroacetamido)pyrazin-2-yl)cyclohexyl)carbamate 1P

Under an atmosphere of argon, trifluoroacetic anhydride was added dropwise to a stirred solution of tert-butyl (4-(5-amino-6-(isobutyramidomethyl)pyrazin-2-yl)cyclohexyl) carbamate 1N (900 mg, 2.3 mmol) and triethylamine (91.3 mL, 9.2 mmol) in anhydrous THF (45 mL). Stirring was continued at RT for 1 h and then the reaction diluted with EtOAc and washed with water, saturated brine, dried (Na₂SO₄) and concentrated in vacuo. The residue was purified by silica-gel chromatography, eluting with 0-60% EtOAc in DCM, to afford the title compound as a white solid (1 g, 89%). LCMS (Method 3): Rₜ=0.97 min, m/z=486.3 [M−H].

### Step 7. tert-Butyl (4-(3-isopropyl-8-(2,2,2-trifluoroacetamido)imidazo[1,5-a]pyrazin-5-yl)cyclohexyl)carbamate 1Q

In a sealed tube under an atmosphere of argon, (methoxycarbonylsulfamoyl) triethyl ammonium hydroxide, inner salt (Burgess reagent) (1.07 g, 4.51 mmol) was added to a stirred solution of tert-butyl (4-(6-(isobutyramidomethyl)-5-(2,2,2-trifluoroacetamido) pyrazin-2-yl)cyclohexyl)carbamate 1P in anhydrous THF (8.5 mL). The stirred reaction was heated to 65° C. for 0.5 h then cooled, concentrated in vacuo and the residue purified by silica-gel chromatography, eluting with 0-35% EtOAc in DCM, to afford the title compound as a white solid (340 mg, 35%). LCMS (Method 3): Rₜ=1.06 min and 1.08 min mixture of cis and trans isomers, respectively, m/z=470.3 [M+H].

### Step 8. tert-Butyl (4-(1-bromo-3-isopropyl-8-(2,2,2-trifluoroacetamido)imidazo[1,5-a] pyrazin-5-yl)cyclohexyl)carbamate 1R

N-Bromosuccinimide (155 mg, 0.87 mmol) was added to a stirred solution of tert-butyl (4-(3-isopropyl-8-(2,2,2-trifluoroacetamido)imidazo[1,5-a]pyrazin-5-yl)cyclohexyl)carbamate 1Q in 1:1 mixture of 2-Me-THF:MeCN (20 mL) at RT. Stirring was continued at RT for 1 h and then the reaction was diluted with 2-Me-THF, washed with water, saturated brine, dried (Na₂SO₄) and concentrated in vacuo. The residue was purified by silica-gel chromatography, eluting with 10% EtOAc in DCM, to afford the title compound as a pale yellow foam (340 mg, 77%). LCMS (Method 2): Rₜ=1.78 min, m/z=548.3 [M (⁷⁹Br)+H].

In another embodiment, amide analogs of the invention such as 2G can be made as outlined in Scheme 3. Quenching of lithiated 8-chloro-3-isopropylimidazo[1,5-a]pyrazine, 2A with carbon dioxide yields the carboxylic acid 2B and amide formation under standard conditions provides an amide such as 2C, which is brominated to provide 2D and then treated with ammonium hydroxide to give 2E. Palladium-catalyzed coupling of 2E with an aryl boronic acid such as 1C gives 2F, and deprotection with a suitable acid yields the final product 2G

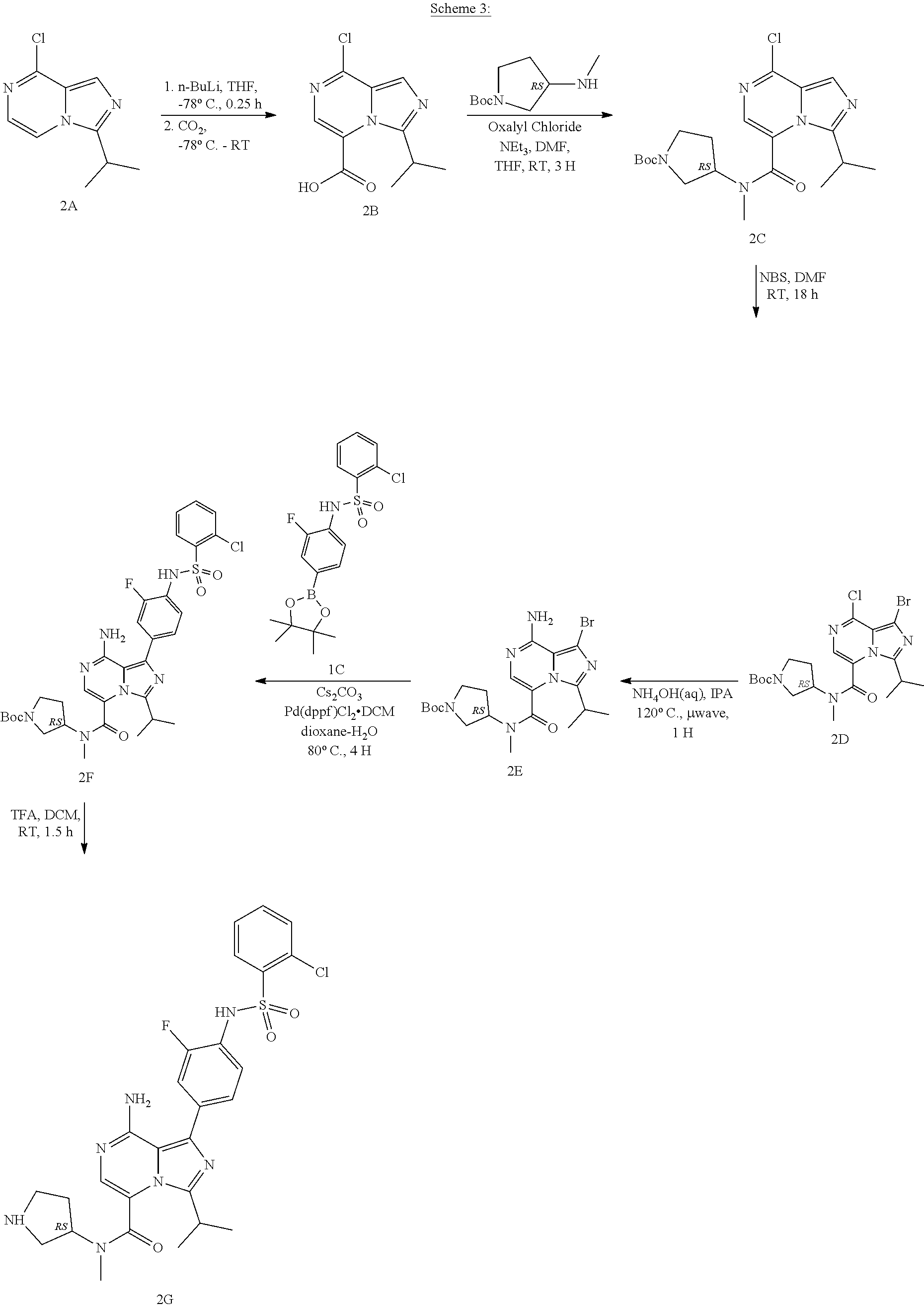

### Example 5: 8-Amino-1-(4-(((2-chlorophenyl)methyl)sulfonamido)-3-fluorophenyl)-3-isopropyl-N-(pyrrolidin-3-yl)imidazo[1,5-a]pyrazine-5-carboxamide, 2G

### Step 1: 8-Chloro-3-isopropylimidazo[1,5-a]pyrazine-5-carboxylic acid 2B

To a solution of 8-chloro-3-isopropylimidazo[1,5-a]pyrazine 2A (10.00 g, 51.11 mmol) in THF (100 mL) at −78° C. under N₂ was added dropwise a solution of n-butyllithium (2.5 M solution in THF, 22.5 mL, 56.22 mmol), keeping the temperature below −60° C. The resulting dark brown solution was stirred at −78° C. for 15 min then solid CO₂ (25.0 g, 568 mmol) was added portion wise and the mixture allowed to warm to 0° C. and poured into an aqueous solution of citric acid. The mixture was diluted with 2-Me-THF and layers separated. The aqueous layer was extracted 2-Me-THF and the combined organic layers were washed water, saturated brine, dried (MgSO₄) and concentrated in vacuo. The residue was triturated with Et₂O to give a dark yellow solid 2B (7.13 g, 58%). LCMS (Method 1): Rₜ=0.95 min, m/z=240.2 [M(³⁵Cl)+H]⁺.

### Step 2: tert-Butyl 3-(8-chloro-3-isopropyl-N-methylimidazo[1,5-a]pyrazine-5-carboxamido)pyrrolidine-1-carboxylate 2C

A stirred solution 8-chloro-3-isopropylimidazo[1,5-a]pyrazine-5-carboxylic acid 2B (500 mg, 2.09 mmol) in THF (20 mL) at RT was treated dropwise with oxalyl chloride (0.18 mL, 2.09 mmol) followed by catalytic amount 1,3-dimethyl-2-imidazolinone (24 mg, 0.209 mmol) and the resulting mixture stirred at RT for 1 h before the addition of a solution tert-butyl-3-(methylamino)pyrrolidine-1-carboxylate (501 mg, 2.50 mmol) and Et₃N (0.58 mL, 4.17 mmol) in THF (10 mL) in one portion. The mixture was stirred at RT for 2 h. After this time the solvent was removed in vacuo, and the residue diluted with a 9:1 mixture of diethyl ether and cyclohexane, and the resulting white precipitate removed by filtration. The filtrate was concentrated in vacuo, and the residue purified by silica-gel chromatography, eluting with 0-100% EtOAc in cyclohexane, to give title compound 2C as a pale yellow gum (385 mg, 44%). LCMS (Method 3): Rₜ=1.46 min, m/z=422.3 [M(³⁵Cl)+H]⁺.

### Step 3: tert-Butyl 3-(1-bromo-8-chloro-3-isopropyl-N-methylimidazo[1,5-a]pyrazine-5-carboxamido)pyrrolidine-1-carboxylate 2D

A stirred solution tert-butyl 3-(8-chloro-3-isopropyl-N-methylimidazo[1,5-a]pyrazine-5-carboxamido)pyrrolidine-1-carboxylate 2C (383 mg, 0.912 mmol) in DMF (10 mL) was treated portion wise with N-bromosuccinimide (162 mg, 0.912 mmol) and the resulting mixture stirred for 18 h at RT. The mixture was diluted EtOAc and brine, and the separated organic layer, dried (MgSO₄) and concentrated in vacuo to give the title product 2D (457 mg, 100%), which was used without further purification. LCMS (Method 4): Rₜ=1.49 min, m/z=500.1 [M(⁷⁹Br)+H].

### Step 4: tert-Butyl 3-(8-amino-1-bromo-3-isopropyl-N-methylimidazo[1,5-a]pyrazine-5-carboxamido)pyrrolidine-1-carboxylate 2E

A mixture tert-butyl 3-(1-bromo-8-chloro-3-isopropyl-N-methylimidazo[1,5-a]pyrazine-5-carboxamido)pyrrolidine-1-carboxylate 2D (457 mg, 0.912 mmol) and 2M NH₃ in IPA (9.1 mL, 18.25 mmol) was sealed in a microwave via and irradiated at 120° C. for 1 h. The solvent was removed in vacuo, and the resulting residue purified silica-gel chromatography, eluting with 0-5% (2M NH₃/MeOH) in DCM, to give the title compound 2E as pale yellow solid (233 mg, 53%). LCMS (Method 3): Rₜ=1.35 min, m/z=479.1 [M (⁷⁹Br)—H].

### Step 5: tert-Butyl 3-(8-amino-1-(4-(((2-chlorophenyl)methyl)sulfonamido)-3-fluorophenyl)-3-isopropyl-N-methylimidazo[1,5-a]pyrazine-5-carboxamido)pyrrolidine-1-carboxylate 2F

A stirred mixture tert-butyl 3-(8-amino-1-bromo-3-isopropyl-N-methylimidazo[1,5-a]pyrazine-5-carboxamido)pyrrolidine-1-carboxylate 2E (233 mg, 0.484 mmol), 1-(2-chlorophenyl)-N-(2-fluoro-4-(4,4,5,5-tetramethyl-1,3,2-dioxaborolan-2-yl)phenyl)methanesulfonamide (239 mg, 0.581 mmol), Pd₂(dppf)Cl₂.DCM (40 mg, 0.048 mmol), caesium carbonate (473 mg, 1.45 mmol), in a mixture of 1,4-dioxane (8 mL) and water (0.7 mL) was sealed and degassed with argon before stirring at 80° C. for 4 h. The mixture was cooled to RT and solvent removed in vacuo. The residue was diluted water and extracted EtOAc (×2). The combined extracts were washed saturated brine, dried (MgSO₄) and concentrated in vacuo to give crude product 2F (442 mg, quantitative), which was used without further purification. LCMS: (Method 3): Rₜ=1.05 min, m/z=686.3 [M(³⁵Cl)+H].

### Step 6: 8-Amino-1-(4-(((2-chlorophenyl)methyl)sulfonamido)-3-fluorophenyl)-3-isopropyl-N-(pyrrolidin-3-yl)imidazo[1,5-a]pyrazine-5-carboxamide 2G (Example 5)

To a stirred solution crude tert-butyl 3-(8-amino-1-(4-(((2-chlorophenyl)methyl)sulfonamido)-3-fluorophenyl)-3-isopropyl-N-methylimidazo[1,5-a]pyrazine-5-carboxamido)pyrrolidine-1-carboxylate 2F (75 mg, 0.109 mmol) in DCM (1 mL) was added TFA (0.17 mL, 2.19 mmol) and the resulting mixture stirred at RT for 1 h. The solvent was removed in vacuo and the residue diluted with MeOH and passed through SCX2 column eluting with NH₃ in MeOH to give the crude product as a dark solid. Further purification by C18 reverse phase cartridge, using 5%-98% MeCN in H₂O (0.1% HCO₂H), gave the title compound 2G a tan solid (12 mg, 18%), LCMS (QC Method 1): Rₜ=2.67 min, m/z 585.9. ¹H NMR (400 MHz, MeOD) δ: 8.52 (1H, br s), 8.07-8.04 (1H, m), 7.61-7.54 (3H, m), 7.45-7.41 (1H, m), 7.35-7.28 (2H, m), 7.16 (1H, br s), 4.86 (3H, s), 4.97-4.70 (1H, m), 3.66-3.03 (5H, m), 2.54-2.09 (2H, m), 1.43-1.19 (6H, m).

Additional compounds of the invention can be made according to the chemistry outlined in Scheme 4. Treatment of 8-chloro-3-isopropylimidazo[1,5-a]pyrazine 2A with n-butyllithium followed by quenching with iodine gives iodo-intermediate 3A, which can undergo Pd(0)-catalyzed coupling with vinyl or aryl boronates to give a coupling product such as 3B. Bromination with a brominating agent, such as NBS, gives the bromide 3C and reaction with ammonium hydroxide, intermediate 3D, which can undergo acid deprotection to afford 3E. Reaction of 3E with a suitable ketone, such as oxetan-3-one, gives 3F and a second Pd(0)-catalyzed coupling with a sulfonamide boronate 3G, as before, gives a coupling product such as 3H.

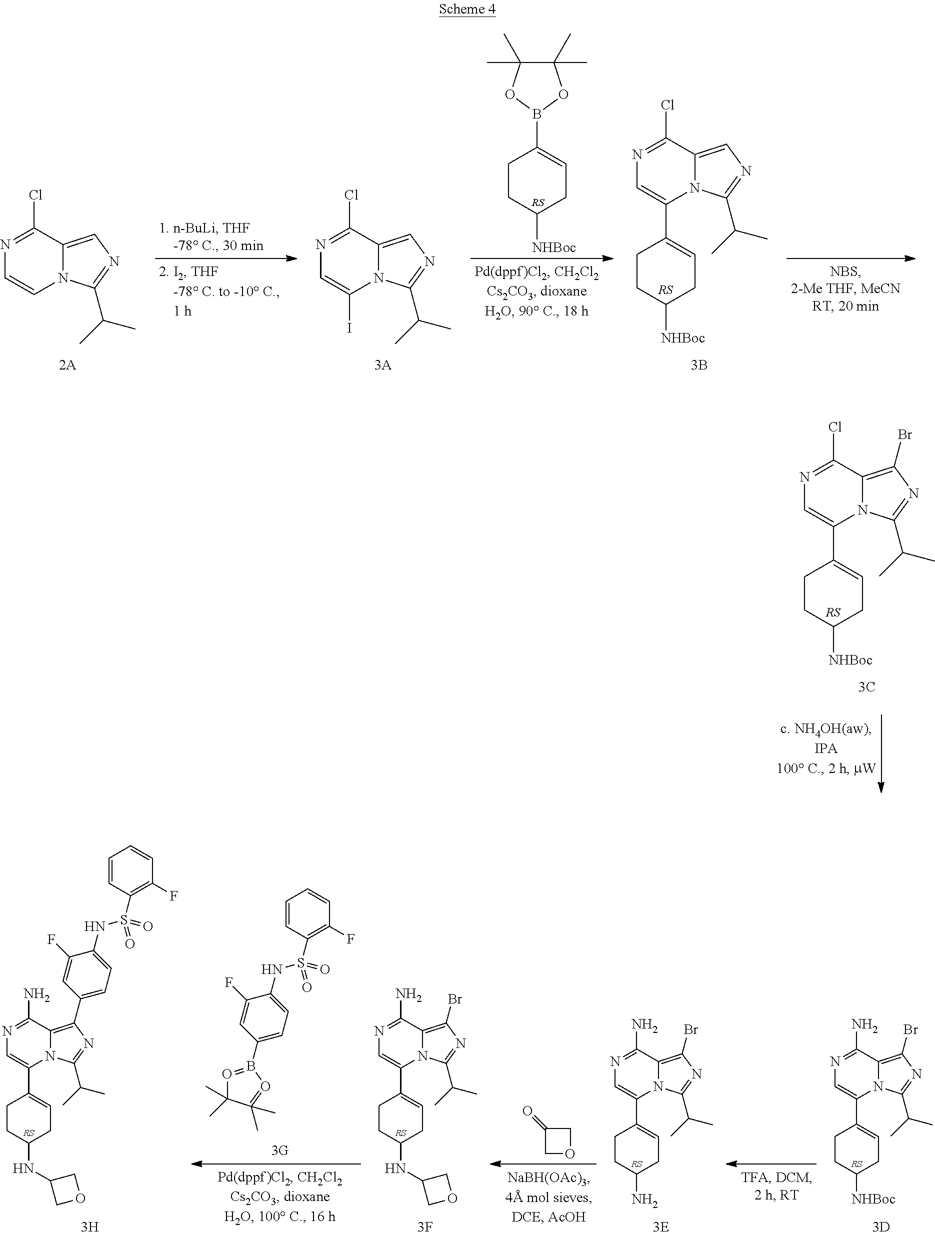

### Example 6: N-(4-(8-Amino-3-isopropyl-5-(4-(oxetan-3-ylamino)cyclohex-1-en-1-yl)imidazo[1,5-a]pyrazin-1-yl)-2-fluorophenyl)-2-fluorobenzenesulfonamide, 3H

### Step 1: 8-Chloro-5-iodo-3-isopropylimidazo[1,5-a]pyrazine 3A

To a stirred solution of 8-chloro-3-isopropylimidazo[1,5-a]pyrazine (31 g, 158.5 mmol) in dry THF (300 mL) at −78° C. under nitrogen was added n-butyllithium (2.5 M in hexanes, 82.4 mL, 206 mmol), keeping the internal temperature below −65° C. After stirring for 30 min a solution of iodine (56.3 g, 221.8 mmol) in THF (50 mL) was added dropwise over 10 min keeping the internal temperature below −65° C. during addition. The resulting suspension was stirred for 1 hour whilst warming to −10° C. then the mixture was quenched by the addition of saturated aqueous NH₄Cl and extracted with 2-Me-THF (×3). The combined organic extracts were washed with water and saturated brine, dried (Na₂SO₄) and concentrated in vacuo. The residue was purified by passage through a silica-gel pad, eluting with 0-20% EtOAc in DCM, to give an orange solid, which was triturated with Et₂O to give the title compound 3A as a yellow solid (41.2 g, 81%). LCMS (Method 4): Rₜ=1.32 min, m/z=322.0 [M+H].

### Step 2: tert-Butyl (4-(8-chloro-3-isopropylimidazo[1,5-a]pyrazin-5-yl)cyclohex-3-en-1-yl)carbamate 3B

A mixture of 8-chloro-5-iodo-3-isopropylimidazo[1,5-a]pyrazine 3A (18.08 g, 56.23 mmol), tert-butyl (4-(4,4,5,5-tetramethyl-1,3,2-dioxaborolan-2-yl)cyclohex-3-en-1-yl)carbamate (20.0 g, 61.87 mmol), PdCl₂(dppf).DCM (2.28 g, 2.74 mmol) and cesium carbonate (53.0 g, 162.7 mmol) in a mixture of 1,4-dioxane (144 mL) and water (36 mL) was purged with argon with sonication prior to being heated to 90° C. for 18 h. The reaction mixture was diluted with water and extracted with EtOAc (×2) and the combined organic extract was washed with brine prior to drying (Na₂SO₄), filtering and concentrating in vacuo. The resultant residue was purified by silica-gel pad, eluting with 0-15% EtOAc in DCM to the title compound 3B as yellow solid (16.7 g, 75%). LCMS (Method 1): Rₜ=1.71 min, m/z=391.2 [M(³⁵Cl)+H].

### Step 3: tert-Butyl (4-(1-bromo-8-chloro-3-isopropylimidazo[1,5-a]pyrazin-5-yl)cyclohex-3-en-1-yl)carbamate 3C

To a stirred suspension of tert-butyl (4-(8-chloro-3-isopropylimidazo[1,5-a]pyrazin-5-yl)cyclohex-3-en-1-yl)carbamate 3B (16.65 g, 42.59 mmol) in a mixture of MeCN (49 mL) and 2-Me-THF (98 mL) was added NBS (9.10 g, 51.13 mmol). After stirring at RT for 2 h, the reaction mixture was diluted with 2-Me-THF (200 mL) and washed with 2M aqueous K₂CO₃, water and saturated brine prior to being dried (Na₂SO₄) and concentrated in vacuo to give the title compound 3C as a buff coloured solid (22.0 g, quantitative). LCMS: (Method 1): Rₜ=1.99 min, m/z=469.0 [M(⁷⁹Br)+H].

### Step 4: tert-Butyl (4-(8-amino-1-bromo-3-isopropylimidazo[1,5-a]pyrazin-5-yl)cyclohex-3-en-1-yl)carbamate 3D

A suspension of tert-butyl (4-(1-bromo-8-chloro-3-isopropylimidazo[1,5-a]pyrazin-5-yl)cyclohex-3-en-1-yl)carbamate 3C (20 g, 42.57 mmol) in a mixture of IPA (110 mL) and concentrated aqueous ammonia (60 mL) was split between 10 microwave vials and each vail was heated under microwave irradiation at 100° C. for 2 h. The combined reaction mixtures were diluted with water and extracted with DCM (×2) and the combined extracts were washed with saturated brine prior to drying (Na₂SO₄) and concentrating in vacuo. The residue was purified by silica-gel chromatography, eluting with 0-10% MeOH in DCM, to give the title compound 3D as a light tan foam (11.61 g, 67%). LCMS: (Method 1): Rₜ=1.12 min, m/z=450.1 [M(⁷⁹Br)+H].

### Step 5: 5-(4-Aminocyclohex-1-en-1-yl)-1-bromo-3-isopropylimidazo[1,5-a]pyrazin-8-amine 3E

A stirred solution of tert-butyl (4-(8-amino-1-bromo-3-isopropylimidazo[1,5-a]pyrazin-5-yl)cyclohex-3-en-1-yl)carbamate 3D was treated with TFA and then allowed to stir at RT for 2 h. The mixture was concentrated in vacuo and the residue diluted with toluene and the evaporation repeated (×2) to give a brown oil. The crude product was applied to an SCX-2 cartridge that had been pre-equilibrated with MeOH. Elution with 2M NH₃ in MeOH gave the semi-pure product as a yellow solid, which was triturated with a mixture of toluene, Et₂O and MTBE to give the title compound 3E as a yellow solid (10.1 g, quantitative). LCMS: (Method 3): Rₜ=1.12 min, m/z=350.1 [M(⁷⁹Br)+H].

### Step 6: 1-Bromo-3-isopropyl-5-(4-(oxetan-3-ylamino)cyclohex-1-en-1-yl)imidazo[1,5-a]pyrazin-8-amine 3F

A solution of 5-(4-aminocyclohex-1-en-1-yl)-1-bromo-3-isopropylimidazo[1,5-a]pyrazin-8-amine 3E (5.0 g, 14.27 mmol), oxetan-3-one (0.96 mL, 14.99 mmol) and acetic acid (1.60 mL, 28.55 mmol) in DCE (60 mL) was treated with 4 Å molecular sieves then portion-wise with sodium triacetoxyborohydride (3.93 g, 18.56 mmol). After stirring at RT for 2 days, further oxetan-3-one (0.48 mL, 7.50 mmol), sodium triacetoxyborohydride (1.47 g, 6.97 mmol) and 4 Å molecular sieves were added, and the mixture stirred for an additional 3 h at RT. The mixture was quenched with water and saturated aqueous with rapid stirring and then extracted with DCM (×2). The aqueous phase was adjusted to pH 7-8 with 1M NaOH then extracted with DCM. This was repeated following adjustment of the resulting aqueous to pH 10, and the organic extracts were combined and concentrated in vacuo. The residue was purified by silica-gel chromatography, eluting with 0-40% MeOH in EtOAc, to give the title compound 3F as an off-white solid (2.53 g, 44%). LCMS: (Method 2) Rₜ=1.81 min, m/z=406.2 [M(⁷⁹Br)+H].

### Step 7: N-(4-(8-Amino-3-isopropyl-5-(4-(oxetan-3-ylamino)cyclohex-1-en-1-yl)imidazo[1,5-a]pyrazin-1-yl)-2-fluorophenyl)-2-fluorobenzenesulfonamide 3H (Example 6)

A mixture of 1-bromo-3-isopropyl-5-(4-(oxetan-3-ylamino)cyclohex-1-en-1-yl)imidazo[1,5-a]pyrazin-8-amine 3F (60 mg, 0.148 mmol), 2-fluoro-N-(2-fluoro-4-(4,4,5,5-tetramethyl-1,3,2-dioxaborolan-2-yl)phenyl)benzenesulfonamide 3G (70 mg, 0.177 mmol), PdCl₂(dppf).DCM (11 mg, 0.015 mmol) and cesium carbonate (92 mg, 0.443 mmol) in a mixture of 1,4-dioxane (1.5 mL) and water (0.3 mL) was purged with argon with sonication, then heated at 100° C. for 16 h. The mixture was cooled, filtered and concentrated in vacuo to remove the 1,4-dioxane, and then diluted with water and extracted with EtOAc (×2). The combined extracts were washed with saturated brine, dried (MgSO₄) and concentrated in vacuo. The residue was purified by preparative HPLC to give the title compound 3H as a yellow solid (13 mg, 15%). LCMS (QC Method 1): Rₜ=2.16 min, m/z=595.0 [M+H]. ¹H NMR (400 MHz, d₆-DMSO) δ: 8.14 (1H, s), 7.79-7.73 (1H, m), 7.71-7.64 (1H, m), 7.45-7.38 (1H, m), 7.36-7.26 (4H, m), 6.69-6.53 (1H, m), 5.88 (3H, d, J=22.2 Hz), 4.69-4.64 (2H, m), 4.36 (2H, t, J=5.9 Hz), 4.08-4.00 (1H, m), 3.51-2.73 (2H, m), 2.41-1.51 (7H, m), 1.39 (3H, d, J=7.0 Hz), 1.12 (3H, d, J=6.2 Hz).

The following examples in Table 1 were prepared using a similar method to Example 6 by replacing the sulfonamide boronate 3G as appropriate. The sulfonamide boronates 3G where either commercially available or were prepared according to Scheme 1, compound 2C.

TABLE 1

| | | | LCMS | HPLC Rₜ |
|---|---|---|---|---|
| | | ¹H NMR data | m/z | (min)/QC |
| Ex. | Structure | (400 MHz, d₆-DMSO) | (M + H) | Method |
| 7 | 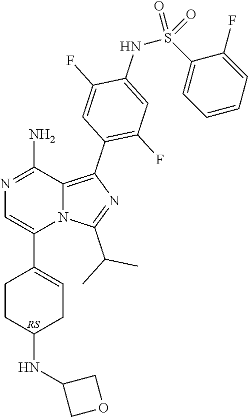 [SMILES: CC(C)c1nc(-c2cc(F)c(NS(=O)(=O)c3ccccc3F)cc2F)c2c(N)ncc(C3=CCC(NC4COC4)CC3)n12] | 8.14 (1H, s), 7.84-7.78 (1H, m), 7.64- 7.58 (1H, m), 7.38-7.29 (2H, m), 7.17- 7.09 (2H, m), 6.61 (1H, s), 5.92 (1H, s), 5.79 (2H, s), 4.73-4.68 (2H, m), 4.48- 4.41 (2H, m), 4.22-4.16 (1H, m), 3.52- 2.99 (2H, m), 2.47-1.49 (6H, m), 1.37 (3H, d, J = 6.6 Hz), 1.11 (3H, d, J = 6.5 Hz) | 613 | 2.48/1 |
| 8 | 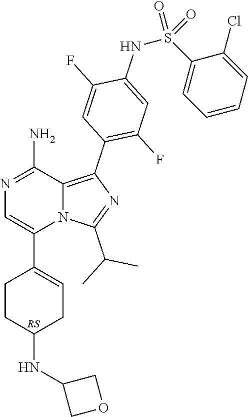 [SMILES: CC(C)c1nc(-c2cc(F)c(NS(=O)(=O)c3ccccc3Cl)cc2F)c2c(N)ncc(C3=CCC(NC4COC4)CC3)n12] | 8.14(1H. s), 8.0-7.98 (1H,m), 7.57- 7.50 (2H, m), 7.46-7.42 (1H, m), 7.14 - 7.02 (2H, m), 6.58 (1H, s), 5.93 (1H, s), 5.80 (2H, s), 4.72-4.66 (2H, m)5 4.46- 4.40 (2H, m), 4.19-4.11 (1H, m), 3.28- 2.86 (2H, m), 2.43-1.44 (6H, m), 1.34 (3H, d, J = 4.6 Hz), 1.10 (3H, d, J = 9.3 Hz) | 629 | 2.51/2 |
| 9 | 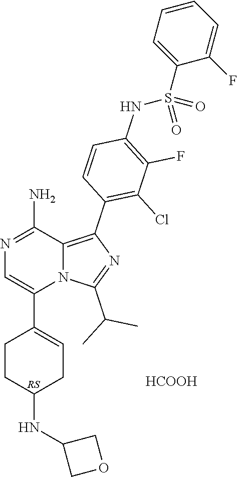 [SMILES: CC(C)c1nc(-c2ccc(NS(=O)(=O)c3ccccc3F)c(F)c2Cl)c2c(N)ncc(C3=CCC(NC4COC4)CC3)n12 ; "HCOOH"] | 8.13 (1H, s), 7.83-7.77 (1H, m), 7.71- 7.64 (1H, m), 7.4 -7.39 (1H, m), 7.38- 7.32 (1H, m), 7.31-7.27 (1H, m), 7.16- 7.13 (1H, m), 6.61 (1H, s), 5.97 (1H, s), 5.71 (2H, s), 4.74-4.68 (2H, m), 4.51- 4.46 (2H, m), 4.27-4.20 (1H, m), 3.43- 2.97 (2H, m), 2.42-1.47 (6H, m), 1.37 (3H, d, J = 5.4 Hz), 1.13 (3H, d, J = 6.4 Hz) | 629 | 2.43/2 |
| 10 | 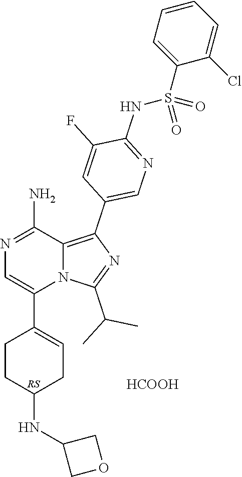 [SMILES: CC(C)c1nc(-c2cnc(NS(=O)(=O)c3ccccc3Cl)c(F)c2)c2c(N)ncc(C3=CCC(NC4COC4)CC3)n12 ; "HCOOH"] | 8.14 (1H, s), 8.12-8.09 (1H, m), 7.89 (1H, s), 7.60 (1H, d, J = 13.1 Hz), 7.54- 7.44 (3H, m), 6.63 (1H, s), 6.04 (2H, s), 5.94 (1H, s), 4.72-4.66 (2H, m), 4.46- 4.40 (2H, m), 4.20-4.12 (1H, m), 3.42- 2.95 (2H, m), 2.43-1.53 (6H, m), 1.39 (3H, d, J = 5.6 Hz), 1.11 (3H, d, J = 7.5 Hz) | 612 | 2.27/1 |
| 11 | 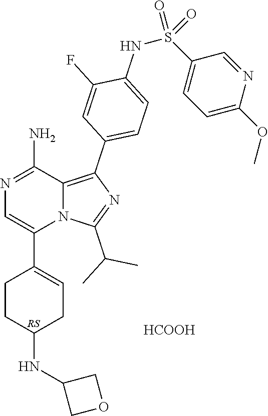 [SMILES: COc1ccc(S(=O)(=O)Nc2ccc(-c3nc(C(C)C)n4c(C5=CCC(NC6COC6)CC5)cnc(N)c34)cc2F)cn1 ; "HCOOH"] | 8.53-8.51 (1H, m), 8.14 (1H, s), 8.03- 7.99 (1H, m), 7.39-7.30 (3H, m), 7.01- 6.97 (1H, m), 6.63 (1H, s), 5.89 (3H, br s), 4.69-4.64 (2H, m), 4.39-4.35 (2H, m), 4.07-4.00 (1H, m), 3.92 (3H, s), 3.44-2.79 (2H, m), 2.40-1.76 (6H, m), 1.38 (3H, d, J = 6.9 Hz), 1.12 (3H, d, J = 6.5 Hz) | 608 | 2.40/1 |
| 12 | 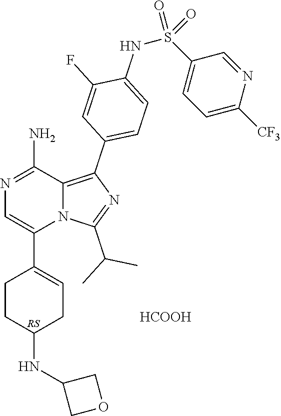 [SMILES: CC(C)c1nc(-c2ccc(NS(=O)(=O)c3ccc(C(F)(F)F)nc3)c(F)c2)c2c(N)ncc(C3=CCC(NC4COC4)CC3)n12 ; "HCOOH"] | 9.08 (1H, m), 8.39 (1H, m), 8.14 (1H, s), 8.09 (1H, m), 7.37-7.32 (1H, m), 7.30-7.23 (2H, m), 6.63 (1H, s), 5.95 (3H, s), 4.73-4.68 (2H, m), 4.48-4.43 (2H, m), 4.22-4.16 (1H, m), 3.44-2.91 (2H, m), 2.46-1.45 (6H, m), 1.37 (3H, d, J = 6.4 Hz), 1.10 (3H, d, J = 6.4 Hz) | 646 | 2.64/1 |
| 13 | 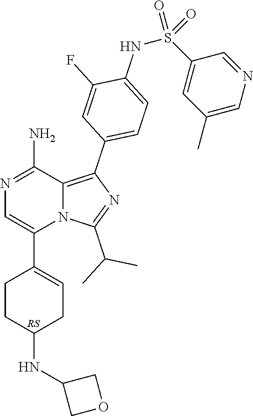 [SMILES: Cc1cncc(S(=O)(=O)Nc2ccc(-c3nc(C(C)C)n4c(C5=CCC(NC6COC6)CC5)cnc(N)c34)cc2F)c1] | 8.70 (1H, s), 8.55 (1H, s), 8.17 (2H, s), 7.92 (1H, s), 7.33-7.27 (1H, m), 7.22- 7.15 (2H, m), 6.61 (1H, s), 5.92 (1H, s), 5.86 (1H, s), 4.69-4.63 (2H, m), 4.39- 4.33 (2H, m), 4.07-3.98 (1H, m), 2.81- 1.42 (7H, m), 2.34 (3H, s), 1.37 (3H, d, = 7.5 Hz), 1.12 (3H, d, J = 5.9 Hz); | 592 | 2.28/1 |
| 14 | 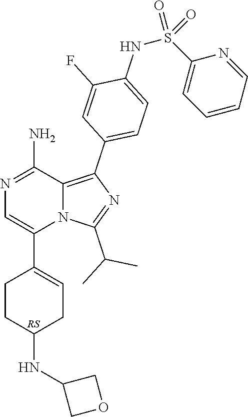 [SMILES: CC(C)c1nc(-c2ccc(NS(=O)(=O)c3ccccn3)c(F)c2)c2c(N)ncc(C3=CCC(NC4COC4)CC3)n12] | 9.46-9.27 (1H, brs), 8.77-8.74 (1H, br s), 8.12-8.07 (1H, m), 7.97 (1H, s), 7.96 (1H, s), 7.72-7.68 (1H, m), 7.47-7.41 (1H, m), 7.40-7.33 (2H, m), 6.66 (1H, s), 6.51 (1H, s), 6.01 (1H, s), 4.83-4.77 (2H, m), 4.71-4.64 (2H, m), 4.61-4.54 (1H, m), 3.46-3.22 (2H, m), 2.61-2.27 (6H, m), 1.42 (3H, d, J = 6.3 Hz), 1.13 (3H, d, J = 6.0 Hz); | 578 | 2.00/2 |
| 15 | 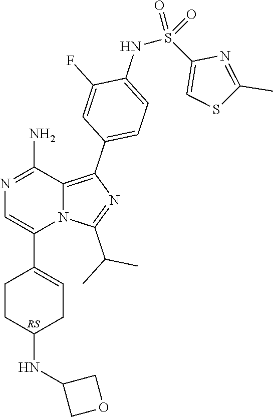 [SMILES: Cc1nc(S(=O)(=O)Nc2ccc(-c3nc(C(C)C)n4c(C5=CCC(NC6COC6)CC5)cnc(N)c34)cc2F)cs1] | 8.19 (1H, s), 8.14 (1H, s), 7.39-7.33 (3H, m), 6.63 (1H, s), 5.90 (3H, s), 4.70-4.64 (2H, m), 4.42-4.36 (2H, m), 4.10-4.04 (1H, m), 3.41-3.22 (1H, m), 2.84-2.80 (1H, m), 2.72 (3H, s), 2.40- 1.47 (7H, m), 1.38 (3H, d, J = 6.7 Hz), 1.12 (3H, d, J = 6.9 Hz); | 598 | 2.05/2 |
| 20 | 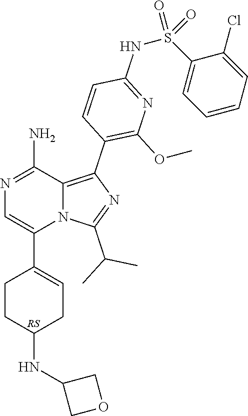 [SMILES: COc1nc(NS(=O)(=O)c2ccccc2Cl)ccc1-c1nc(C(C)C)n2c(C3=CCC(NC4COC4)CC3)cnc(N)c12] | 8.27-8.23 (1H, m), 7.63-7.59 (2H, m), 7.58-7.51 (2H, m), 6.58 (1H, d, J = 7.9 Hz), 6.53 (1H, s), 5.89 (1H, s), 5.61 (2H, s), 4.69-4.62 (2H, m), 4.35 (2H, t, J = 6.1 Hz), 4.02 (1H, quint, J = 6.9 Hz), 3.42 (3H, s), 2.82-2.74 (1H, m), 2.37-2.17 (3H, m), 2.13-2.03 (1H, m), 1.99-1.80 (2H, m), 1.60-1.38 (1H, m), 1.34 (3H, d, J = 5.0 Hz), 1.07 (3H, d, J = 6.5 Hz). | 624/626 | 2.44/1 |
| 21 | 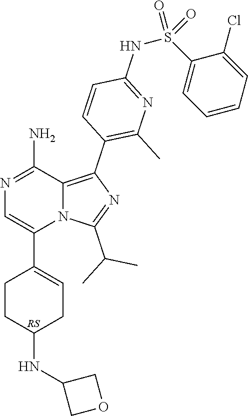 [SMILES: Cc1nc(NS(=O)(=O)c2ccccc2Cl)ccc1-c1nc(C(C)C)n2c(C3=CCC(NC4COC4)CC3)cnc(N)c12] | 8.19 (1H, s), 8.15-8.11 (1H, m), 7.59 (1H, d, J = 9.0 Hz), 7.57-7.49 (3H, m), 7.07 (1H, d, J = 9.0 Hz), 6.58 (1H, s), 5.94-5.85 (3H, m), 4.69-4.62 (2H, m), 4.39-4.32 (2H, m), 4.01 (1H, quint, J = 6.8 Hz), 2.82-2.70 (1H, m), 2.39- 2.27 (2H, m), 2.24 (3H, s), 2.13-2.04 (1H, m), 1.93-1.75 (2H, m), 1.59-1.40 (1H, m), 1.34 (3H, d, J = 6.5 Hz), 1.09 (3H, d, J = 6.4 Hz). | 608/610 | 2.22/1 |

Example 7: N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-fluorobenzenesulfonamide (R and S enantiomers)
Example 8: N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-chlorobenzenesulfonamide (R and S enantiomers)
Example 9: N-(4-(8-Amino-3-isopropyl-5-(4-(oxetan-3-ylamino)cyclohex-1-en-1-yl)imidazo[1,5-a]pyrazin-1-yl)-3-chloro-2-fluorophenyl)-2-fluorobenzenesulfonamide (R and S enantiomers)
Example 10: N-(5-(8-Amino-3-isopropyl-5-(4-(oxetan-3-ylamino)cyclohex-1-en-1-yl)imidazo[1,5-a]pyrazin-1-yl)-3-fluoropyridin-2-yl)-2-chlorobenzenesulfonamide (R and S enantiomers)
Example 11: N-(4-(8-Amino-3-isopropyl-5-(4-(oxetan-3-ylamino)cyclohex-1-en-1-yl)imidazo[1,5-a]pyrazin-1-yl)-2-fluorophenyl)-6-methoxypyridine-3-sulfonamide (R and S enantiomers)
Example 12: N-(4-(8-Amino-3-isopropyl-5-(4-(oxetan-3-ylamino)cyclohex-1-en-1-yl)imidazo[1,5-a]pyrazin-1-yl)-2-fluorophenyl)-6-(trifluoromethyl)pyridine-3-sulfonamide (R and S enantiomers)
Example 13: N-(4-(8-Amino-3-isopropyl-5-(4-(oxetan-3-ylamino)cyclohex-1-en-1-yl)imidazo[1,5-a]pyrazin-1-yl)-2-fluorophenyl)-5-methylpyridine-3-sulfonamide (R and S enantiomers)
Example 14: N-(4-(8-Amino-3-isopropyl-5-(4-(oxetan-3-ylamino)cyclohex-1-en-1-yl)imidazo[1,5-a]pyrazin-1-yl)-2-fluorophenyl)pyridine-2-sulfonamide (R and S enantiomers)
Example 15: N-(4-(8-Amino-3-isopropyl-5-(4-(oxetan-3-ylamino)cyclohex-1-en-1-yl)imidazo[1,5-a]pyrazin-1-yl)-2-fluorophenyl)-2-methylthiazole-4-sulfonamide (R and S enantiomers)
Example 20: N-(5-(8-amino-3-isopropyl-5-(4-(oxetan-3-ylamino)cyclohex-1-en-1-yl)imidazo[1,5-a]pyrazin-1-yl)-6-methoxypyridin-2-yl)-2-chlorobenzenesulfonamide (R and S enantiomers)
Example 21: N-(5-(8-amino-3-isopropyl-5-(4-(oxetan-3-ylamino)cyclohex-1-en-1-yl)imidazo[1,5-a]pyrazin-1-yl)-6-methylpyridin-2-yl)-2-chlorobenzenesulfonamide (R and S enantiomers)

Other compounds of the invention can be made according to the chemistry outlined in Scheme 5. 3D can undergo a Pd(0)-catalyzed coupling with an aryl boronate such as 1C to give 4A. Acid deprotection affords the product 4B and further reaction with a suitable alkylating agent such as methyl vinyl sulfone, the final product 4C. Alternatively, intermediate 3D can be converted into 3E using an acid such as TFA, and can then be converted into the morpholine 4D by reacting with an alkylating agent such as 1-bromo-2-(2-bromoethoxy)ethane. 4D can undergo a Pd(0)-catalyzed coupling with an aryl boronate such as 1C to give the final product 4E. Alternatively, intermediate 3E can be converted to converted into ether 4F by reacting with an alkylating agent such as 2-methoxyethyl trifluoromethanesulfonate. 4F can undergo a Pd(0)-catalyzed coupling with an aryl boronate such as 1C to give the final product 4G.

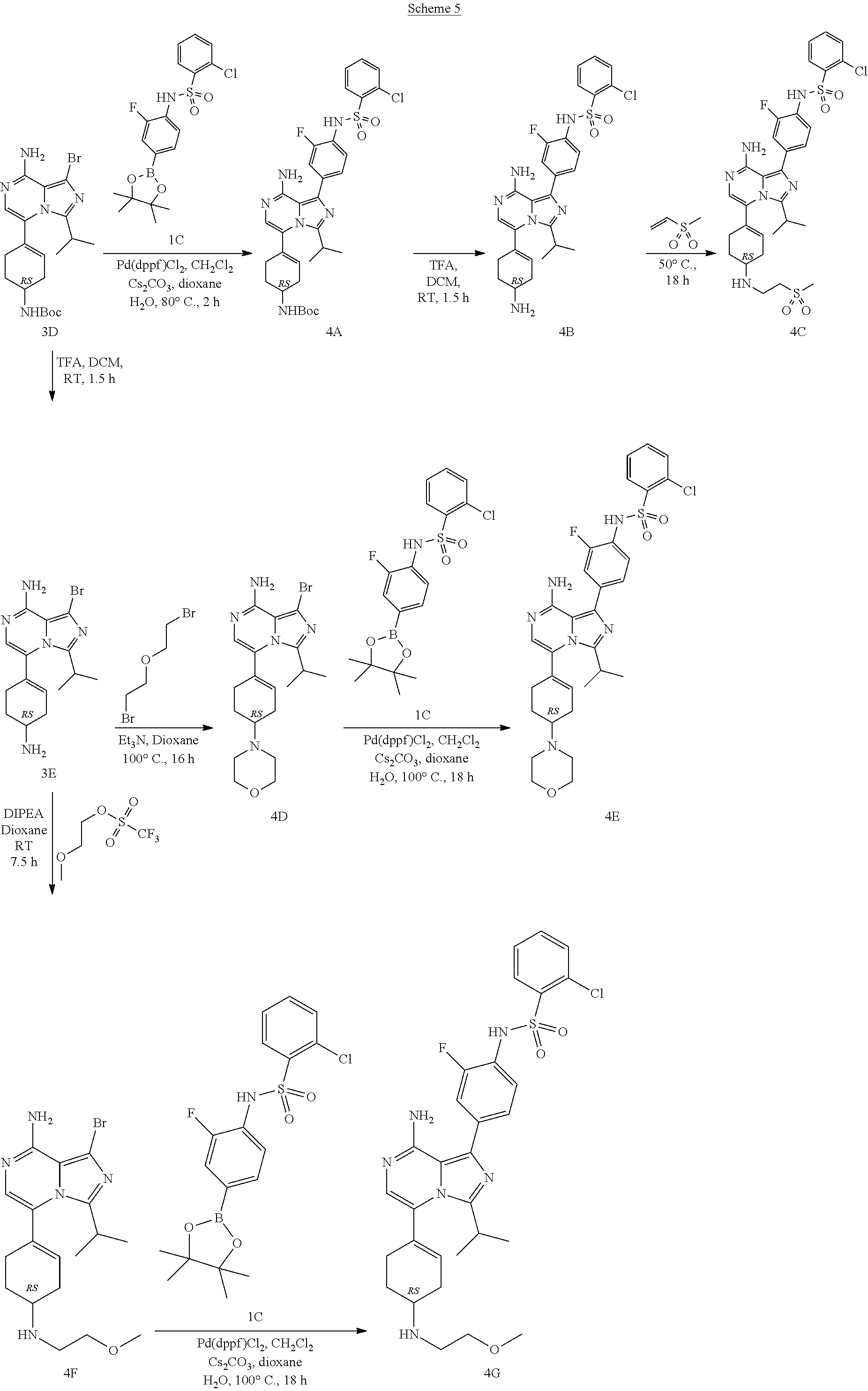

### Example 16: N-(4-(8-Amino-3-isopropyl-5-(4-((2-(methylsulfonyl)ethyl)amino)cyclohex-1-en-1-yl)imidazo[1,5-a]pyrazin-1-yl)-2-fluorophenyl)-2-chlorobenzenesulfonamide, 4C

### Step 1: tert-Butyl (4-(8-amino-1-(4-((2-chlorophenyl)sulfonamido)-3-fluorophenyl)-3-isopropylimidazo[1,5-a]pyrazin-5-yl)cyclohex-3-en-1-yl)carbamate 4A

A mixture of tert-butyl (4-(8-amino-1-bromo-3-isopropylimidazo[1,5-a]pyrazin-5-yl)cyclohex-3-en-1-yl)carbamate 3D (1.54 g, 3.42 mmol), 2-chloro-N-(2-fluoro-4-(4,4,5,5-tetramethyl-1,3,2-dioxaborolan-2-yl)phenyl) benzenesulfonamide 1C (1.69 g, 4.10 mmol), PdCl₂(dppf).DCM (0.28 g, 0.342 mmol) and cesium carbonate (3.34 g, 10.26 mmol) in a mixture of 1,4-dioxane (32 mL) and water (8 mL) was purged with argon with sonication, then heated at 80° C. for 2 h. The mixture was cooled to RT, diluted with 10% citric acid and extracted with EtOAc (×2). The combined extracts were washed with brine, dried (MgSO₄) and concentrated in vacuo. The residue was purified by silica-gel chromatography, eluting with 0-100% EtOAc in cyclohexane, to give the title compound 4A as a brown solid (1.15 g, 51%). LCMS (Method 4): Rₜ=1.09 min, m/z=655.3 [M(³⁵Cl)+H].

### Step 2: N-(4-(8-Amino-5-(4-aminocyclohex-1-en-1-yl)-3-isopropylimidazo[1,5-a]pyrazin-1-yl)-2-fluorophenyl)-2-chlorobenzenesulfonamide 4B

To a solution of tert-butyl (4-(8-amino-1-(4-((2-chlorophenyl)sulfonamido)-3-fluorophenyl)-3-isopropylimidazo[1,5-a]pyrazin-5-yl)cyclohex-3-en-1-yl)carbamate 4A (1.15 g, 1.76 mmol) in DCM (18 mL) was added TFA (2 mL) and the resulting mixture was stirred at RT for 2 h. The solvents were evaporated in vacuo and the residue was dissolved in MeOH and applied to an SCX-2 cartridge that had been pre-equilibrated with DCM. Elution with 2M NH₃ in MeOH gave the crude product, which was triturated with MeCN/Et₂O to give the title compound 4B as tan coloured solid (950 mg, 98%). LCMS (Method 4): Rₜ=0.71 min, m/z=555.1 [M(³⁵Cl)+H]⁺.

### Step 3: N-(4-(8-Amino-3-isopropyl-5-(4-((2-(methylsulfonyl)ethyl)amino)cyclohex-1-en-1-yl)imidazo[1,5-a]pyrazin-1-yl)-2-fluorophenyl)-2-chlorobenzenesulfonamide, 4C (Example 16)

A stirred solution of N-(4-(8-amino-5-(4-aminocyclohex-1-en-1-yl)-3-isopropylimidazo[1,5-a]pyrazin-1-yl)-2-fluorophenyl)-2-chlorobenzenesulfonamide 4B (100 mg, 0.180 mmol) in MeOH (2 mL) was treated sequentially with methyl vinyl sulfone (19 μL, 0.216 mmol) and 2-fluoroethyl trifluoromethanesulfonate (20 μL, 0.159 mmol) and the resulting mixture stirred at 50° C. for 24 h. The mixture was cooled, concentrated in vacuo and the residue purified using silica-gel chromatography, eluting with 0-10% (2M NH₃/MeOH) in DCM, to give semi-pure product, which was further purified by MDAP, giving the title compound as a yellow solid (47 mg, 37%). LCMS (QC Method 2): Rₜ=3.36 min, m/z=661.0 [M(³⁵Cl)+H]. ¹H NMR (400 MHz, d₆-DMSO) δ: 10.77 (1H, s), 9.05 (2H, s), 8.01 (1H, dd, J=1.5, 7.6 Hz), 7.73-7.65 (2H, m), 7.56-7.51 (1H, m), 7.46-7.36 (3H, m), 6.71 (1H, s), 6.13 (1H, s), 3.60-3.43 (5H, m), 3.2-3.20 (1H, m), 3.17 (3H, s), 2.74-2.66 (1H, m), 2.43-2.23 (4H, m), 1.76-1.66 (1H, m), 1.39 (3H, d, J=5.4 Hz), 1.16 (3H, d, J=7.3 Hz).

### Example 17: N-(4-(8-Amino-5-(4-aminocyclohex-1-en-1-yl)-3-isopropylimidazo[1,5-a]pyrazin-1-yl)-2-fluorophenyl)-1-(2-methylthiazol-4-yl)methanesulfonamide

The title compound was prepared from intermediate 3D using an analogous method to that employed to prepare compound 4B (Scheme 5).

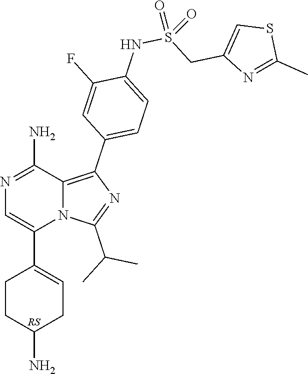 [SMILES: Cc1nc(CS(=O)(=O)Nc2ccc(-c3nc(C(C)C)n4c(C5=CCC(N)CC5)cnc(N)c34)cc2F)cs1]

LCMS (QC Method 1): Rₜ=2.13 min, m/z=555.9 [M+H]. H NMR (400 MHz d₆-DMSO) δ: 8.30 (1H, br s), 7.46 (1H, t, J=8.7 Hz), 7.43 (1H, s), 7.32 (1H, dd, J=11.8 and 1.9 Hz), 7.24 (1H, dd, J=8.1 and 1.9 Hz), 6.68 (1H, s), 6.04-5.93 (3H, m), 4.49 (2H, s), 3.36 (2H, m), 2.61 (3H, s), 2.46-1.68 (6H, m), 1.48-1.36 (3H, m), 1.19-1.11 (3H, m).

### Example 18: N-(4-(8-Amino-3-isopropyl-5-(4-morpholinocyclohex-1-en-1-yl)imidazo[1,5-a]pyrazin-1-yl)-2-fluorophenyl)-2-chlorobenzenesulfonamide, 4E

### Step 1: N-(4-(8-Amino-3-isopropyl-5-(4-morpholinocyclohex-1-en-1-yl)imidazo[1,5-a] pyrazin-1-yl)-2-fluorophenyl)-2-chlorobenzenesulfonamide 4D

A solution of 5-(4-aminocyclohex-1-en-1-yl)-1-bromo-3-isopropylimidazo[1,5-a]pyrazin-8-amine 3E (100 mg, 0.285 mmol), 1-bromo-2-(2-bromoethoxy)ethane (66 mg, 0.285 mmol) and Et₃N (80 μL, 0.571 mmol) in 1,4-dioxane (3.0 mL) was heated to 100° C. for 16 h. The mixture was cooled to RT, diluted with saturated aqueous NaHCO₃ and extracted with EtOAc (×2). The combined extracts were washed with saturated brine, dried (MgSO₄), and concentrated in vacuo. The residue was purified by silica-gel chromatography, eluting with 0-7% (7 M NH₃ in MeOH) in DCM, to give the title compound 4D as a white solid (38 mg, 32%). LCMS (Method 3): Rₜ=1.20 min, m/z=420.2 [M(⁷⁹Br)+H]⁺.

### Step 2: N-(4-(8-Amino-3-isopropyl-5-(4-morpholinocyclohex-1-en-1-yl)imidazo[1,5-a]pyrazin-1-yl)-2-fluorophenyl)-2-chlorobenzenesulfonamide, 4E (Example 18)

The title compound was prepared from intermediate 4D and 1C using an analogous method to prepare compound 3H (Scheme 4). LCMS (QC Method 1): Rₜ=2.66 min, m/z=625.1 [M+H]. ¹H NMR (400 MHz d₆-DMSO) δ: 7.96 (1H, dd, J=8.0 and 1.5 Hz), 7.66-7.58 (2H, m), 7.48 (1H, t, J=7.9 Hz), 7.33-7.22 (3H, m), 6.63 (1H, s) 5.98-5.94 (1H, m), 5.86 (2H, s), 3.64-3.57 (5H, m), 2.61-1.96 (8H, m), 1.55-1.07 (9H, m).

### Example 19: N-(4-(8-amino-3-isopropyl-5-(4-((2-methoxyethyl)amino)cyclohex-1-en-1-yl)imidazo[1,5-a]pyrazin-1-yl)-2-fluorophenyl)-2-chlorobenzenesulfonamide

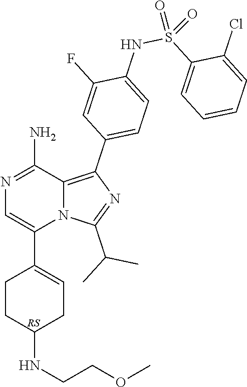 [SMILES: COCCNC1CC=C(c2cnc(N)c3c(-c4ccc(NS(=O)(=O)c5ccccc5Cl)c(F)c4)nc(C(C)C)n23)CC1]

### Step 1: 1-Bromo-3-isopropyl-5-(4-((2-methoxyethyl)amino)cyclohex-1-en-1-yl)imidazo[1,5-a]pyrazin-8-amine 4F

A solution of 2-methoxyethyl trifluoromethanesulfonate (CAS: 112981-50-7; 45 mg, 0.214 mmol) in dry 1,4-dioxane (3 mL) was added dropwise to a solution of 5-(4-aminocyclohex-1-en-1-yl)-1-bromo-3-isopropylimidazo[1,5-a]pyrazin-8-amine 3E (150 mg, 0.428 mmol) and DIPEA (149 μL, 0.856 mmol) in 1,4-dioxane (7.0 mL) at RT and the mixture stirred for 1.5 h. Further 2-methoxyethyl trifluoromethanesulfonate (23 mg, 0.110 mmol) was added and the mixture stirred for 6 h. The mixture was partitioned between water (5 mL) and DCM and the aqueous phase extracted with DCM. The combined extracts were passed through a phase separating cartridge and concentrated in vacuo. The residue was purified by chromatography on silica (12 g), eluting with 0-7% (7 M NH₃ in MeOH) in DCM, to give the title compound (76 mg, 87%) LCMS (Method 3): Rₜ=1.20 min, m/z=408/410 [M+H].

### Step 2: N-(4-(8-amino-3-isopropyl-5-(4-((2-methoxyethyl)amino)cyclohex-1-en-1-yl)imidazo[1,5-a]pyrazin-1-yl)-2-fluorophenyl)-2-chlorobenzenesulfonamide

The title compound was prepared from intermediate 4F using an analogous method to that employed to prepare compound 4E (Scheme 5).

LCMS (QC Method 1): Rₜ=2.71 min, m/z=613.0/615 [M+H]. ¹H NMR (400 MHz d₆-DMSO) δ: 8.21 (1H, s), 7.96 (1H, dd, J=7.9 and 1.8 Hz), 7.46-7.44 (1H, m), 7.41-7.33 (2H, m), 7.17 (1H, t, J=8.7 Hz), 7.09 (1H, dd, J=12.1 and 2.1 Hz), 6.94 (1H, d, J=8.4 Hz), 6.58 (1H, s), 5.92 (1H, br s), 5.83 (1H, br s), 3.47-3.43 (1H, M), 2.86-2.79 (5H, m), 2.54 (3H, s), 2.18-1.80 (6H, m), 1.31-1.07 (6H, m).

### Example 22: N-(4-(8-amino-5-(4-dimethylamino)cyclohex-1-en-1-yl)-3-isopropylimidazo[1,5-a]pyrazin-1-yl)-2-fluorophenyl)-1-(2-chlorophenyl)methanesulfonamide

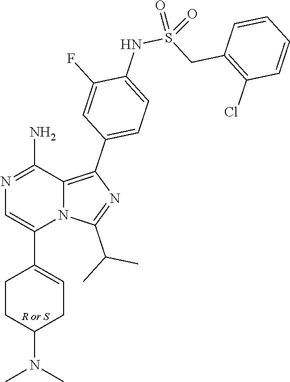 [SMILES: CC(C)c1nc(-c2ccc(NS(=O)(=O)Cc3ccccc3Cl)c(F)c2)c2c(N)ncc(C3=CCC(N(C)C)CC3)n12]

A mixture of N-(4-(8-amino-5-(4-methylamino)cyclohex-1-en-1-yl)-3-isopropylimidazo[1,5-a]pyrazin-1-yl)-2-fluorophenyl)-1-(2-chlorophenyl)methanesulfonamide (CAS: 2294035-27-9; 100 mg, 0.171 mmol), paraformaldehyde (26 mg, 0.857 mmol) and formic acid (0.032 mL, 0.857 mmol) in methanol (2 mL) was sonicated to aid solubility, then heated at 60° C. for 18 h. The mixture cooled to RT and treated with sodium hydroxide (35 mg, 0.875 mmol) to give a yellow colored solution. The solution was concentrated in vacuo, diluted with water and extracted with 2-Me-THF. The combined extracts were washed with saturated brine, dried (Na₂SO₄) and concentrated in vacuo to give a yellow solid. Purification by chromatography on silica, eluting with DCM/2M ammonia in methanol (0-20%), follow by MDAP gave the title compound as an off-white solid (15.6 mg, 15%). LCMS (QC Method 1): Rₜ=2.73 min, m/z=597/599 [M(+H]. ¹H NMR (400 MHz, DMSO) δ: 7.55-7.51 (1H, m), 7.48 (1H, dd, J=8.7, 8.7 Hz), 7.45-7.42 (1H, m), 7.33-7.30 (2H, m), 7.23 (1H, dd, J=1.9, 12.0 Hz), 7.15 (1H, dd, J=1.7, 8.2 Hz), 6.64 (1H, s), 5.99-5.96 (1H, m), 5.94 (2H, s), 4.45-4.42 (2H, m), 2.42-2.32 (2H, m), 2.27 (6H, s), 2.18-2.10 (2H, m), 2.01 (1H, d, J=11.7 Hz), 1.58-1.49 (1H, m), 1.43 (3H, d, J=6.5 Hz), 1.14 (3H, d, J=6.3 Hz).

### Example 23

### N-(5-(8-amino-3-isopropyl-5-(4-(oxetan-3-ylamino)cyclohex-1-en-1-yl)imidazo[1,5-a]pyrazin-1-yl)-3-fluoropyridin-2-yl)-2-fluorobenzenesulfonamide

### Example 24

### N-(4-(8-amino-3-isopropyl-5-(4-(oxetan-3-ylamino)cyclohex-1-en-1-yl)imidazo[1,5-a]pyrazin-1-yl)-2-fluorophenyl)-5-chloropyridine-2-sulfonamide

### Example 25

### N-(4-(8-amino-3-isopropyl-5-(4-(oxetan-3-ylamino)cyclohex-1-en-1-yl)imidazo[1,5-a]pyrazin-1-yl)-2-fluorophenyl)-2-methyloxazole-5-sulfonamide

### Example 26: Pharmacological in vitro Assay

Biochemical Assay: Inhibition of RNase activity of IRE1α

The RNase reactions were performed in 384 well black ProxiPlate-384 Plus plates (PERKIN Elmer) using 50 mM Tris assay buffer with 0.5 mM MgCl₂, 10 mM KCl, 0.03% Tween, 2 mM DTT and 1% DMSO. Test compounds were prepared on the day of assay and dispensed using D300 digital dispenser as a 10-point ½ log dilution series in duplicate, normalized to a final DMSO concentration of 4%. Test compounds were pre-incubated for 30 min at room temperature with IRE1α kinase (E31-11G from Signal Chem) in 2.5 μL of assay buffer. Then 2.5 μl of assay buffer containing substrate (5′ Alexa Fluor 647-rCrArU rGrUrC rCrGrC rArGrC rGrCrArUrG-Iowa Black RQ quencher 3′) added, giving a final concentration of enzyme of 0.325 nM and of substrate of 100 nM. After 20 minutes incubation at room temperature the reactions were stopped by added 5 μL of 5M urea, incubated at room temperature for 10 minutes and fluorescence measured on a plate reader (EnVision, PerkinElmer) IC₅₀ values calculated by fitting a sigmoidal curve to percent inhibition of control versus compound concentration.

### Example 27: Cellular In Vitro Assay

### Cellular XBP1 Splicing Assay

ARPE-19 cells stably expressing XBP1 (a.a. 1-376) with nano-luciferase gene sequence linked so it is in frame when XBP1 is spliced, were cultured in F12 media, 10% FBS, 0.044% sodium bicarbonate, 150 μg/ml hygromycin B and seeded for assays at 5,000 cells in 384 well plates in culture media without hygromycin B and incubated at 37° C./5% CO₂. After overnight incubation test compounds were added to the cell plate in a 10-point ½ log dilution series in duplicate (final DMSO concentration 0.117%). After further incubation of 30 minutes thapsigargin was added (final concentration 150 nM) and then another 4 hour incubation. A NanoLuc luciferase assay (Promega) was used according to the manufacturer's instructions to detect the luciferase and luminescence measured on a luminometer (EnVision, PerkinElmer). IC₅₀ values calculated by fitting a sigmoidal curve to percent inhibition of control of compound concentration.

TABLE 2

| | In vitro assay | Cellular assay |
|---|---|---|
| Example | IC₅₀ (μM) | IC₅₀ (μM) |
| 1 | 0.29 | 0.51 |
| 2 | 0.015 | 0.015 |
| 3 | 0.28 | 0.30 |
| 4 | 0.0064 | 0.005 |
| 5 | 0.13 | >10 |
| 6 | 0.15 | 0.098 |
| 7 | 0.15 | 0.54 |
| 8 | 0.11 | 0.17 |
| 9 | 0.069 | 0.76 |
| 10 | 0.061 | 7.7 |
| 11 | 0.42 | 3.1 |
| 12 | 0.14 | 6.6 |
| 13 | 0.36 | 6.4 |
| 14 | not tested | 5.4 |
| 15 | 0.48 | 6.5 |
| 16 | 0.011 | 0.11 |
| 17 | 0.040 | 7.45 |
| 18 | 0.082 | 0.085 |
| 19 | 0.044 | 0.041 |
| 20 | >1 | >10 |
| 21 | >1 | 6.1 |
| 22 | 0.0015 | 0.0017 |

In vitro assay data are for the inhibition of RNase activity of IRE1α. Cellular assay (IC₅₀ data) are for the XBP1 splicing assay.

### Enumerated Embodiments

The following exemplary embodiments are provided, the numbering of which is not to be construed as designating levels of importance:

Embodiment 1 provides a compound of formula (I), or a salt, solvate, enantiomer, diastereoisomer, isotopologue, or tautomer thereof:

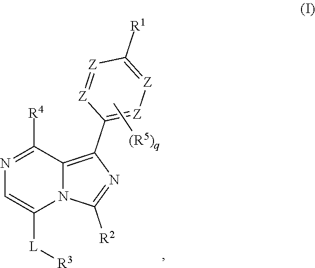

wherein:

R¹ is

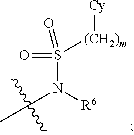

R² is selected from the group consisting of H, methyl, ethyl, propyl, CF₃, CHF₂, cyclopropyl, 1-methylcyclopropyl, isopropyl, tert-butyl, and C₃-C₈ cycloalkyl;

L is selected from the group consisting of a bond, —C(═O)NH, and —C(═O)N(C₁-C₆ alkyl);

R³ is selected from the group consisting of optionally substituted C₁-C₈ alkyl, optionally substituted C₃-C₈ cycloalkyl, optionally substituted C₂-C₈ alkenyl, optionally substituted C₃-C₈ cycloalkenyl, and optionally substituted C₂-C₈ alkynyl;

R⁴ is selected from the group consisting of —NH₂ and —NHR⁸;

R⁵, Z, and q are selected such that the compound of formula (I) is selected from the group consisting of:

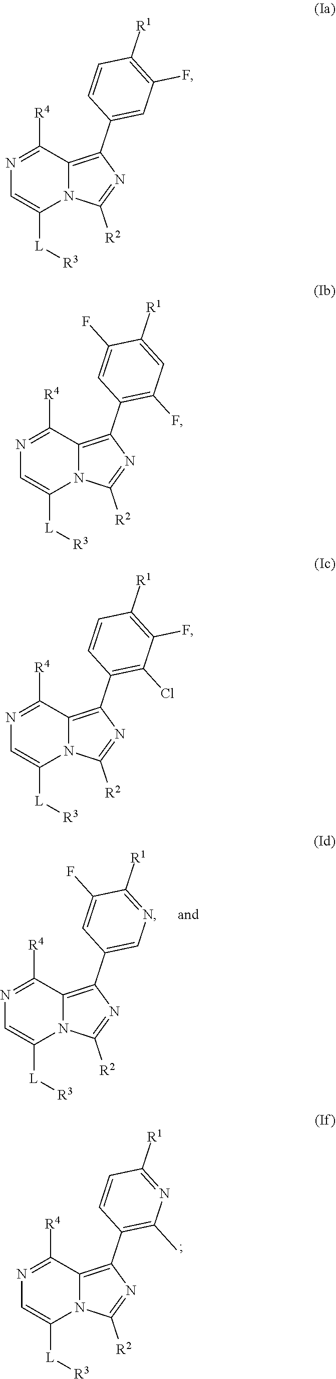

R⁶ is selected from the group consisting of H and optionally substituted C₁-C₆ alkyl;

R⁸ is optionally substituted C₁-C₃ alkyl;

Cy is selected from the group consisting of phenyl, naphthyl, and heteroaryl;
wherein Cy is substituted with 0 to ‘n’ instances of X, each instance of X being independently selected from the group consisting of H, OH, halide, nitrile, optionally substituted C₁-C₆ alkyl, C₁-C₆ haloalkyl, optionally substituted C₁-C₆ alkoxy, optionally substituted phenyl, optionally substituted heteroaryl, and

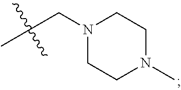 [SMILES: *CN1CCN(C)CC1]

m is an integer selected from the group consisting of 0, 1, 2, 3, and 4; and

n is an integer selected from the group consisting of 0, 1, 2, 3, 4, and 5.

Embodiment 2 provides the compound of Embodiment 1, wherein each occurrence of optionally substituted alkyl, alkenyl, alkynyl, cycloalkyl, or cycloalkenyl is independently optionally substituted with at least one substituent selected from the group consisting of C₁-C₆ alkyl, halo, —OR^{a}, optionally substituted phenyl, optionally substituted heteroaryl, optionally substituted heterocyclyl, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

Embodiment 3 provides the compound of any of Embodiments 1-2, wherein each occurrence of optionally substituted phenyl, naphthyl, or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R^{b})(R^{b}), —NO₂, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl.

Embodiment 4 provides the compound of any of Embodiments 1-3, wherein each occurrence of optionally substituted phenyl, naphthyl, or heteroaryl is independently optionally substituted with at least one substituent selected from the group consisting of C₁-C₆ alkyl, C₁-C₆ haloalkyl, C₁-C₆ haloalkoxy, halo, —CN, —OR^{c}, —N(R^{c})(R^{c}), and C₁-C₆ alkoxycarbonyl, wherein each occurrence of R^{c} is independently H, C₁-C₆ alkyl, or C₃-C₈ cycloalkyl.

Embodiment 5 provides the compound of any of Embodiments 1-4, wherein R¹ is selected from the group consisting of:

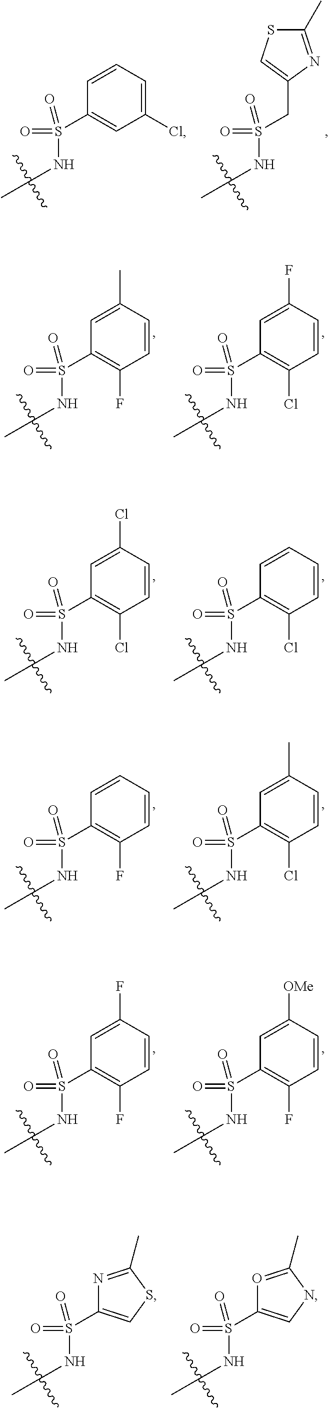

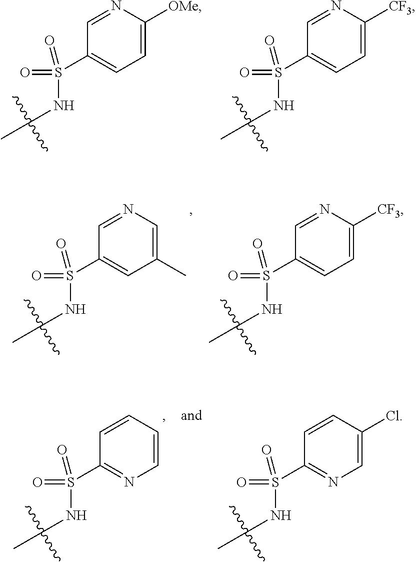 [SMILES: *NS(=O)(=O)c1ccc(OC)nc1 ; *NS(=O)(=O)c1ccc(C(F)(F)F)nc1 ; *NS(=O)(=O)c1cncc(C)c1 ; *NS(=O)(=O)c1ccc(C(F)(F)F)nc1 ; *NS(=O)(=O)c1ccccn1 ; *NS(=O)(=O)c1ccc(Cl)cn1]

Embodiment 6 provides the compound of any of Embodiments 1-5, wherein R² is isopropyl.

Embodiment 7 provides the compound of any of Embodiments 1-6, wherein L-R³ is selected from the group consisting of:

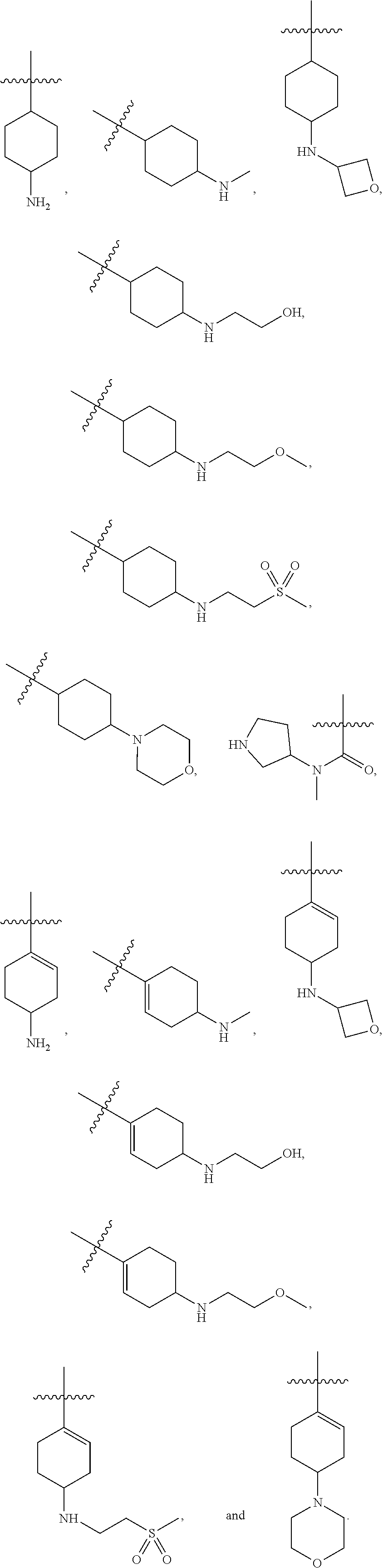

Embodiment 8 provides the compound of any of Embodiments 1-7, wherein L-R³ is selected from the group consisting of:

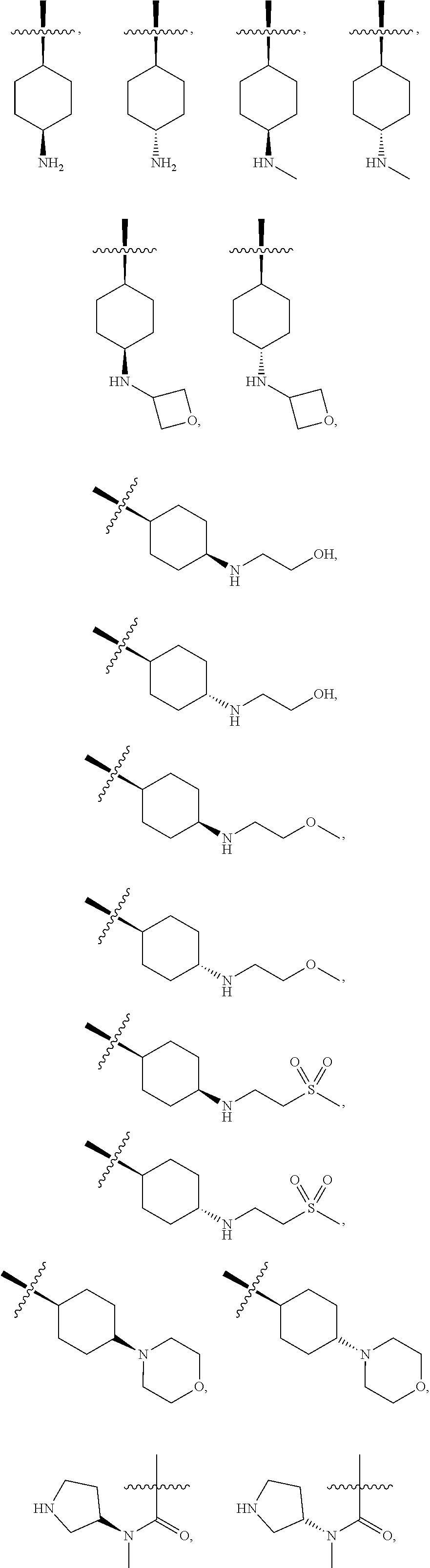

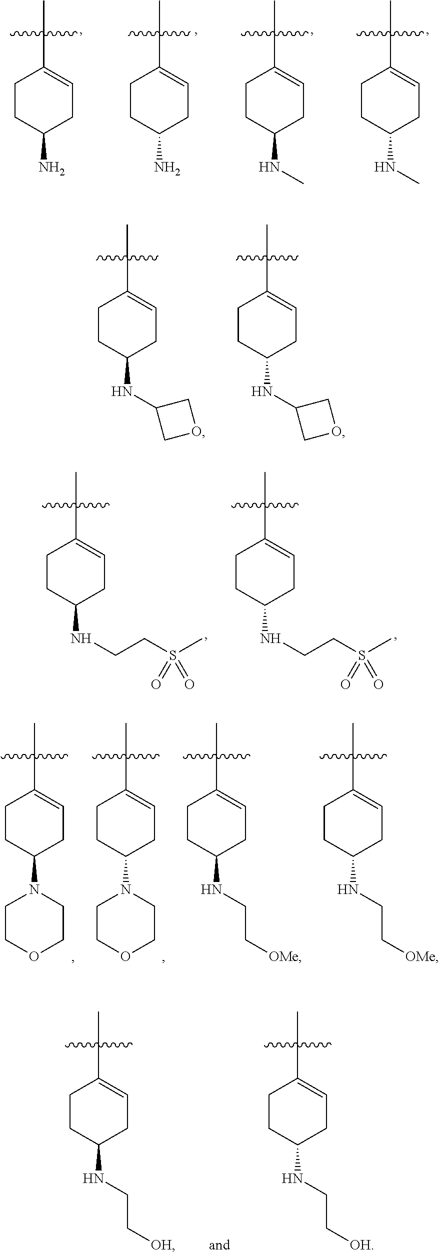

Embodiment 9 provides the compound of any of Embodiments 1-8, wherein R⁴ is —NH₂.

Embodiment 10 provides the compound of any of Embodiments 1-9, which is selected from the group consisting of:
N-(4-(8-Amino-5-((1s,4s)-4-aminocyclohexyl)-3-isopropylimidazo[1,5-a]pyrazin-1-yl)-2-fluorophenyl)-2-chlorobenzenesulfonamide (cis isomer);
N-(4-(8-Amino-5-((1r,4r)-4-aminocyclohexyl)-3-isopropylimidazo[1,5-a]pyrazin-1-yl)-2-fluorophenyl)-2-chlorobenzenesulfonamide (trans isomer);
N-(4-(8-Amino-3-isopropyl-5-((1s,4s)-4-(oxetan-3-ylamino) cyclohexyl)imidazo[1,5-a]pyrazin-1-yl)-2-fluorophenyl)-2-chlorobenzenesulfonamide (cis isomer);
N-(4-(8-Amino-3-isopropyl-5-((1r,4r)-4-(oxetan-3-ylamino) cyclohexyl)imidazo[1,5-a]pyrazin-1-yl)-2-fluorophenyl)-2-chloro benzene sulfonamide (trans isomer);
8-Amino-1-(4-(((2-chlorophenyl)methyl)sulfonamido)-3-fluorophenyl)-3-isopropyl-N-(pyrrolidin-3-yl)imidazo[1,5-a]pyrazine-5-carboxamide;
N-(4-(8-Amino-3-isopropyl-5-(4-(oxetan-3-ylamino)cyclohex-1-en-1-yl)imidazo[1,5-a]pyrazin-1-yl)-2-fluorophenyl)-2-chlorobenzenesulfonamide;
N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-fluorobenzenesulfonamide;
N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-chlorobenzenesulfonamide;
N-(4-(8-Amino-3-isopropyl-5-(4-(oxetan-3-ylamino)cyclohex-1-en-1-yl) imidazo[1,5-a]pyrazin-1-yl)-3-chloro-2-fluorophenyl)-2-fluorobenzenesulfonamide;
N-(5-(8-Amino-3-isopropyl-5-(4-(oxetan-3-ylamino)cyclohex-1-en-1-yl) imidazo[1,5-a]pyrazin-1-yl)-3-fluoropyridin-2-yl)-2-chlorobenzenesulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)-6-methoxypyridine-3-sulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)-6-(trifluoromethyl)pyridine-3-sulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)-4-methylpyridine-2-sulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)pyridine-2-sulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)-2-methylthiazole-4-sulfonamide;
N-(4-(8-Amino-3-isopropyl-5-(4-((2-(methylsulfonyl)ethyl)amino)cyclohex-1-en-1-yl)imidazo[1,5-a]pyrazin-1-yl)-2-fluorophenyl)-2-chlorobenzenesulfonamide;
N-(4-(8-Amino-5-(4-aminocyclohex-1-en-1-yl)-3-isopropylimidazo[1,5-a]pyrazin-1-yl)-2-fluorophenyl)-1-(2-methylthiazol-4-yl)methanesulfonamide;
N-(4-(8-Amino-3-isopropyl-5-(4-morpholinocyclohex-1-en-1-yl)imidazo[1,5-a]pyrazin-1-yl)-2-fluorophenyl)-2-chlorobenzenesulfonamide;
N-(4-(8-amino-3-isopropyl-5-(4-((2-methoxyethyl)amino)cyclohex-1-en-1-yl)imidazo[1,5-a]pyrazin-1-yl)-2-fluorophenyl)-2-chlorobenzenesulfonamide;
N-(5-(8-amino-3-isopropyl-5-(4-(oxetan-3-ylamino)cyclohex-1-en-1-yl)imidazo[1,5-a]pyrazin-1-yl)-6-methoxypyridin-2-yl)-2-chlorobenzenesulfonamide;
N-(5-(8-amino-3-isopropyl-5-(4-(oxetan-3-ylamino)cyclohex-1-en-1-yl)imidazo[1,5-a]pyrazin-1-yl)-6-methylpyridin-2-yl)-2-chlorobenzenesulfonamide;
N-(4-(8-amino-5-(4-dimethylamino)cyclohex-1-en-1-yl)-3-isopropylimidazo[1,5-a]pyrazin-1-yl)-2-fluorophenyl)-1-(2-chlorophenyl)methanesulfonamide;
N-(5-(8-amino-3-isopropyl-5-(4-(oxetan-3-ylamino)cyclohex-1-en-1-yl)imidazo[1,5-a]pyrazin-1-yl)-3-fluoropyridin-2-yl)-2-fluorobenzenesulfonamide;
N-(4-(8-amino-3-isopropyl-5-(4-(oxetan-3-ylamino)cyclohex-1-en-1-yl)imidazo[1,5-a]pyrazin-1-yl)-2-fluorophenyl)-5-chloropyridine-2-sulfonamide;
N-(4-(8-amino-3-isopropyl-5-(4-(oxetan-3-ylamino)cyclohex-1-en-1-yl)imidazo[1,5-a]pyrazin-1-yl)-2-fluorophenyl)-2-methyloxazole-5-sulfonamide;
   or a salt, solvate, enantiomer, diastereoisomer, isotopologue or tautomer thereof.

Embodiment 11 provides a pharmaceutical composition comprising at least one compound of any of Embodiments 1-10 and at least one pharmaceutically acceptable carrier.

Embodiment 12 provides a method of treating a IRE1α-related disease in a subject, the method comprising administering to the subject a therapeutically effective amount of a compound, or a pharmaceutically acceptable salt, solvate, enantiomer, diastereoisomer, or tautomer thereof, of any of Embodiments 1-10 and/or the pharmaceutical composition of Embodiment 11.

Embodiment 13 provides the method of Embodiment 12, wherein the disease is selected from the group consisting of a neurodegenerative disease, a demyelinating disease, cancer, an eye disease, a fibrotic disease, and diabetes.

Embodiment 14 provides the method of any of Embodiments 12-13, wherein the neurodegenerative disease is selected from the group consisting of retinitis pigmentosa, amyotrophic lateral sclerosis, retinal degeneration, macular degeneration, Parkinson's Disease, Alzheimer's Disease, Huntington's Disease, Prion Disease, Creutzfeldt-Jakob Disease, and Kuru.

Embodiment 15 provides the method of any of Embodiments 12-13, wherein the demyelinating disease is selected from the group consisting of Wolfram Syndrome, Pelizaeus-Merzbacher Disease, Transverse Myelitis, Charcot-Marie-Tooth Disease, and Multiple Sclerosis.

Embodiment 16 provides the method of any of Embodiments 12-13, wherein the cancer is multiple myeloma.

Embodiment 17 provides the method of any of Embodiments 12-13, wherein the diabetes is selected from the group consisting of type I diabetes and type II diabetes.

Embodiment 18 provides the method of any of Embodiments 12-13, wherein the eye disease is selected from the group consisting of retinitis pigmentosa, retinal degeneration, macular degeneration, and Wolfram Syndrome.

Embodiment 19 provides the method of any of Embodiments 12-13, wherein the fibrotic disease is selected from the group consisting of idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis.

Embodiment 20 provides a method of inhibiting the activity of an IRE1 protein, the method comprising contacting the IRE1 protein with an effective amount of a compound, or a pharmaceutically acceptable salt thereof, of any of Embodiments 1-10 and/or the pharmaceutical composition of Embodiment 11.

Embodiment 21 provides the method of Embodiment 20, wherein the activity is selected from the group consisting of kinase activity, oligomerization activity, and RNase activity.

Embodiment 22 provides the method of any of Embodiments 20-21, wherein the IRE1 protein is within a cell.

Embodiment 23 provides the method of Embodiment 22, wherein apoptosis of the cell is prevented or minimized.

Embodiment 24 provides the method of any of Embodiments 22-23, wherein the cell is in an organism that has an IRE1α-related disease or disorder.

Embodiment 25 provides the method of any of Embodiments 20-24, wherein the disease or disorder is a neurodegenerative disease, demyelinating disease, cancer, eye disease, fibrotic disease, or diabetes.

Embodiment 26 provides the method of any of Embodiments 12-25, wherein the subject is need of the treatment.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (I), or a salt, solvate, enantiomer, diastereoisomer, isotopologue, or tautomer thereof: 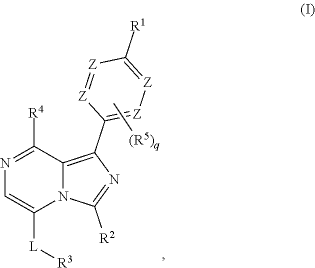
wherein:
R¹ is 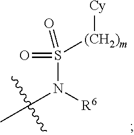
R² is selected from the group consisting of H, methyl, ethyl, propyl, CF₃, CHF₂, cyclopropyl, 1-methylcyclopropyl, isopropyl, tert-butyl, and C₃-C₈ cycloalkyl;
L is selected from the group consisting of a bond, —C(═O)NH, and —C(═O)N(C₁-C₆ alkyl);
R³ is selected from the group consisting of optionally substituted C₁-C₈ alkyl, optionally substituted C₃-C₈ cycloalkyl, optionally substituted C₂-C₈ alkenyl, optionally substituted C₃-C₈ cycloalkenyl, and optionally substituted C₂-C₈ alkynyl;
R⁴ is selected from the group consisting of —NH₂ and —NHR⁸;
R⁵, Z, and q are selected such that (I) is selected from the group consisting of: 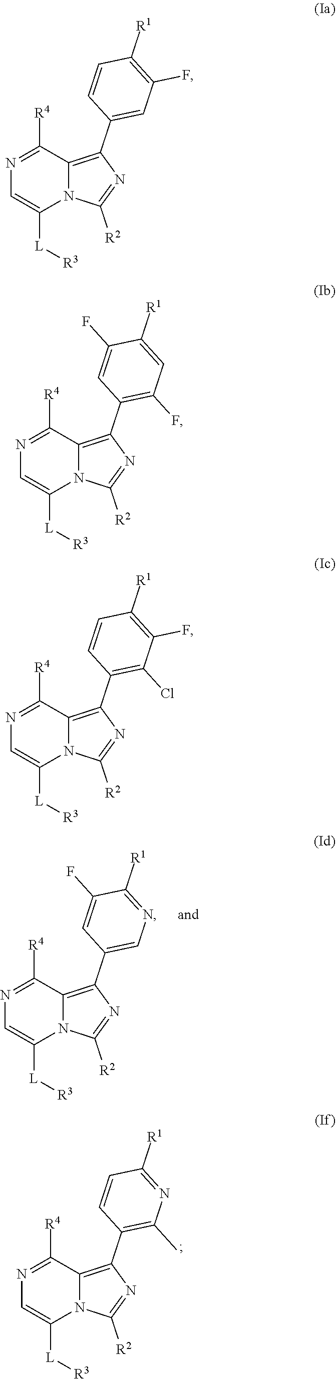
R⁶ is selected from the group consisting of H and optionally substituted C₁-C₆ alkyl;
R⁸ is optionally substituted C₁-C₃ alkyl;
Cy is selected from the group consisting of phenyl, naphthyl, and heteroaryl;
wherein Cy is substituted with 0 to ‘n’ instances of X, each instance of X being independently selected from the group consisting of H, OH, halide, nitrile, optionally substituted C₁-C₆ alkyl, C₁-C₆ haloalkyl, optionally substituted C₁-C₆ alkoxy, optionally substituted phenyl, optionally substituted heteroaryl, and 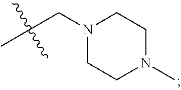 [SMILES: *CN1CCN(C)CC1]
m is an integer selected from the group consisting of 0, 1, 2, 3, and 4; and
n is an integer selected from the group consisting of 0, 1, 2, 3, 4, and 5.

2. The compound of claim 1, wherein each occurrence of optionally substituted alkyl, alkenyl, alkynyl, cycloalkyl, or cycloalkenyl is independently optionally substituted with at least one substituent selected from the group consisting of C₁-C₆ alkyl, halo, —OR^{a}, optionally substituted phenyl, optionally substituted heteroaryl, optionally substituted heterocyclyl, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

3. The compound of claim 1, wherein each occurrence of optionally substituted phenyl, naphthyl, or heteroaryl is independently optionally substituted with at least one substituent selected from the group consisting of C₁-C₆ alkyl, C₁-C₆ haloalkyl, C₁-C₆ haloalkoxy, halo, —CN, —OR^{b}, —N(R^{b})(R^{b}), —NO₂, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl.

4. The compound of claim 1, wherein each occurrence of optionally substituted phenyl, naphthyl, or heteroaryl is independently optionally substituted with at least one substituent selected from the group consisting of C₁-C₆ alkyl, C₁-C₆ haloalkyl, C₁-C₆ haloalkoxy, halo, —CN, —OR^{c}, —N(R^{c})(R^{c}), and C₁-C₆ alkoxycarbonyl, wherein each occurrence of R^{c} is independently H, C₁-C₆ alkyl, or C₃-C₈ cycloalkyl.

5. The compound of claim 1, wherein R¹ is selected from the group consisting of:

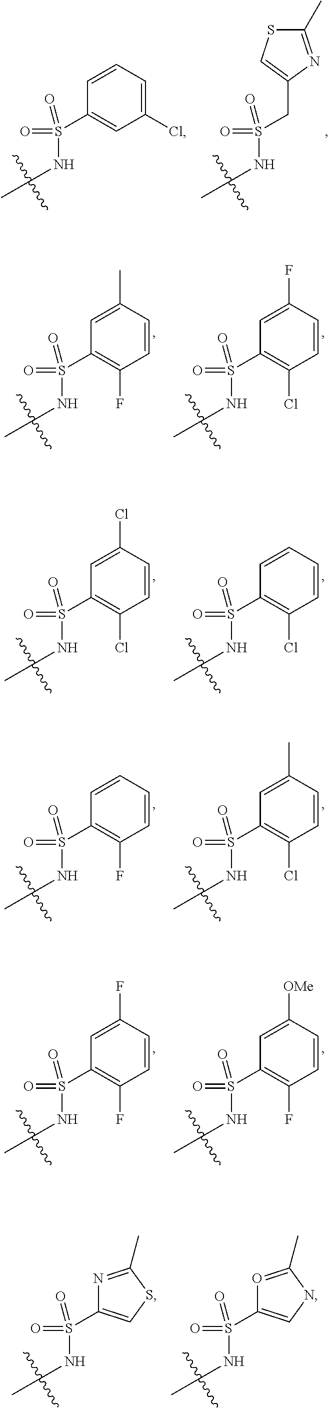

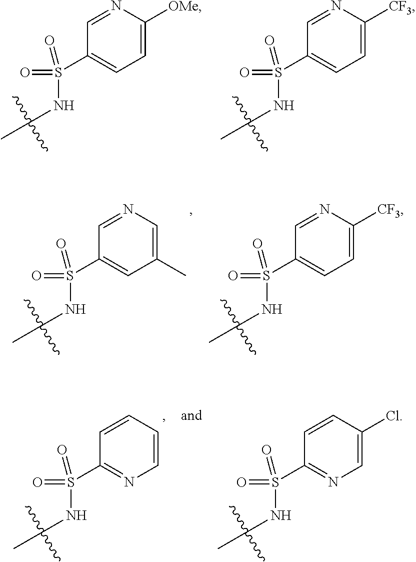 [SMILES: *NS(=O)(=O)c1ccc(OC)nc1 ; *NS(=O)(=O)c1ccc(C(F)(F)F)nc1 ; *NS(=O)(=O)c1cncc(C)c1 ; *NS(=O)(=O)c1ccc(C(F)(F)F)nc1 ; *NS(=O)(=O)c1ccccn1 ; *NS(=O)(=O)c1ccc(Cl)cn1]

6. The compound of claim 1, wherein R² is isopropyl.

7. The compound of claim 1, wherein L-R³ is selected from the group consisting of:

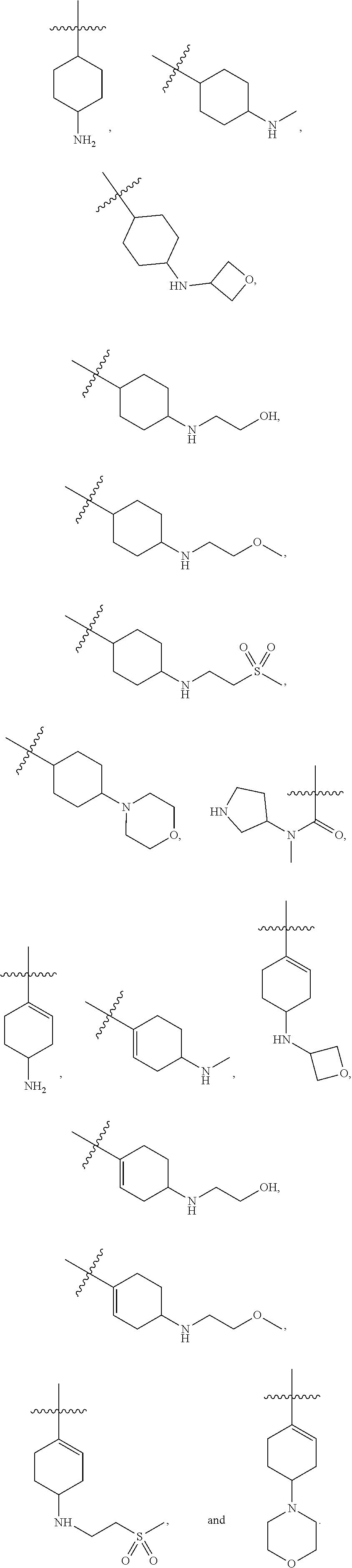

8. The compound of claim 1, wherein L-R³ is selected from the group consisting of:

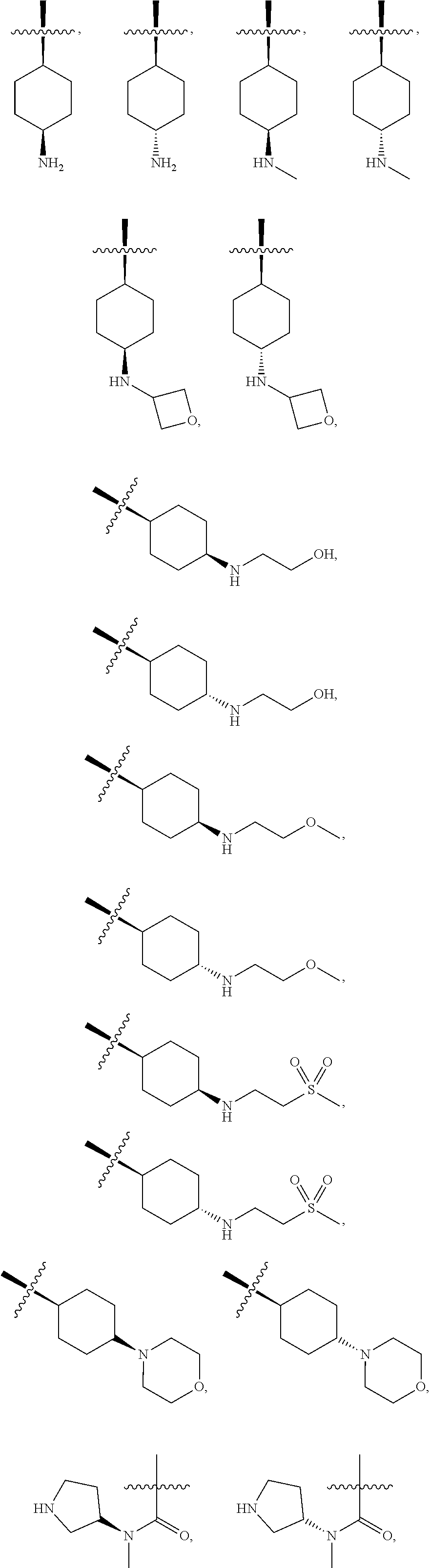

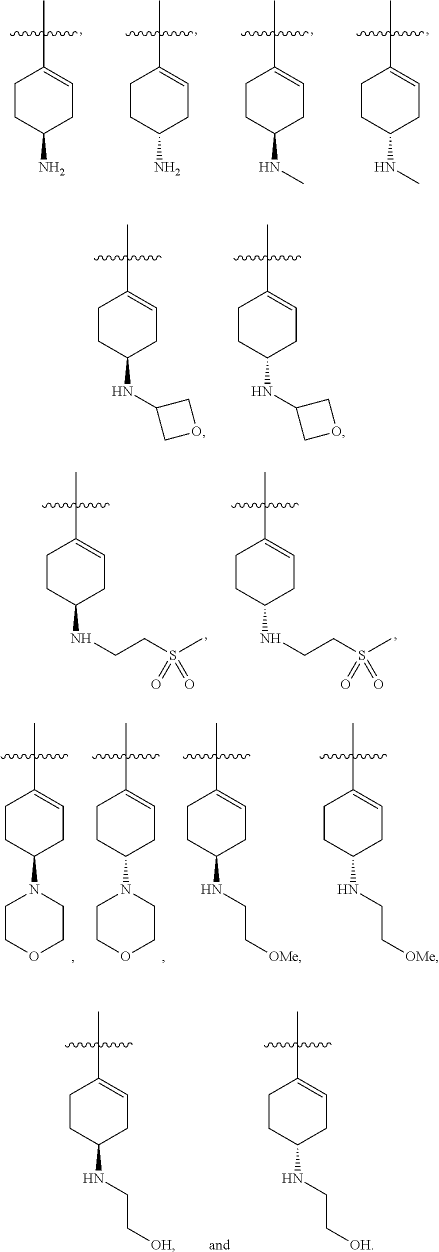

9. The compound of claim 1, wherein R⁴ is —NH₂.

10. The compound of claim 1, which is selected from the group consisting of:
N-(4-(8-Amino-5-((1s,4s)-4-aminocyclohexyl)-3-isopropylimidazo[1,5-a]pyrazin-1-yl)-2-fluorophenyl)-2-chlorobenzenesulfonamide (cis isomer);
N-(4-(8-Amino-5-((1r,4r)-4-aminocyclohexyl)-3-isopropylimidazo[1,5-a]pyrazin-1-yl)-2-fluorophenyl)-2-chlorobenzenesulfonamide (trans isomer);
N-(4-(8-Amino-3-isopropyl-5-((1s,4s)-4-(oxetan-3-ylamino) cyclohexyl)imidazo[1,5-a]pyrazin-1-yl)-2-fluorophenyl)-2-chlorobenzenesulfonamide (cis isomer);
N-(4-(8-Amino-3-isopropyl-5-((1r,4r)-4-(oxetan-3-ylamino) cyclohexyl)imidazo[1,5-a]pyrazin-1-yl)-2-fluorophenyl)-2-chloro benzene sulfonamide (trans isomer);
8-Amino-1-(4-(((2-chlorophenyl)methyl)sulfonamido)-3-fluorophenyl)-3-isopropyl-N-(pyrrolidin-3-yl)imidazo[1,5-a]pyrazine-5-carboxamide;
N-(4-(8-Amino-3-isopropyl-5-(4-(oxetan-3-ylamino)cyclohex-1-en-1-yl)imidazo[1,5-a]pyrazin-1-yl)-2-fluorophenyl)-2-chlorobenzenesulfonamide;
N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-fluorobenzenesulfonamide;
N-(4-(8-Amino-3-isopropyl-5-(4-(oxetan-3-ylamino)cyclohex-1-en-1-yl)imidazo[1,5-a]pyrazin-1-yl)-2,5-difluorophenyl)-2-chlorobenzenesulfonamide;
N-(4-(8-Amino-3-isopropyl-5-(4-(oxetan-3-ylamino)cyclohex-1-en-1-yl) imidazo[1,5-a]pyrazin-1-yl)-3-chloro-2-fluorophenyl)-2-fluorobenzenesulfonamide;
N-(5-(8-Amino-3-isopropyl-5-(4-(oxetan-3-ylamino)cyclohex-1-en-1-yl) imidazo[1,5-a]pyrazin-1-yl)-3-fluoropyridin-2-yl)-2-chlorobenzenesulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)-6-methoxypyridine-3-sulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)-6-(trifluoromethyl)pyridine-3-sulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)-4-methylpyridine-2-sulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)pyridine-2-sulfonamide;
N-(4-(8-Amino-3-isopropyl-5-(4-(oxetan-3-ylamino)cyclohex-1-en-1-yl) imidazo[1,5-a]pyrazin-1-yl)-2-fluorophenyl)-2-methylthiazole-4-sulfonamide;
N-(4-(8-Amino-3-isopropyl-5-(4-((2-(methylsulfonyl)ethyl)amino)cyclohex-1-en-1-yl)imidazo[1,5-a]pyrazin-1-yl)-2-fluorophenyl)-2-chlorobenzenesulfonamide;
N-(4-(8-Amino-5-(4-aminocyclohex-1-en-1-yl)-3-isopropylimidazo[1,5-a]pyrazin-1-yl)-2-fluorophenyl)-1-(2-methylthiazol-4-yl)methanesulfonamide;
N-(4-(8-Amino-3-isopropyl-5-(4-morpholinocyclohex-1-en-1-yl)imidazo[1,5-a]pyrazin-1-yl)-2-fluorophenyl)-2-chlorobenzenesulfonamide;
N-(4-(8-amino-3-isopropyl-5-(4-((2-methoxyethyl)amino)cyclohex-1-en-1-yl)imidazo[1,5-a]pyrazin-1-yl)-2-fluorophenyl)-2-chlorobenzenesulfonamide;
N-(5-(8-amino-3-isopropyl-5-(4-(oxetan-3-ylamino)cyclohex-1-en-1-yl)imidazo[1,5-a]pyrazin-1-yl)-6-methoxypyridin-2-yl)-2-chlorobenzenesulfonamide;
N-(5-(8-amino-3-isopropyl-5-(4-(oxetan-3-ylamino)cyclohex-1-en-1-yl)imidazo[1,5-a]pyrazin-1-yl)-6-methylpyridin-2-yl)-2-chlorobenzenesulfonamide;
N-(4-(8-amino-5-(4-dimethylamino)cyclohex-1-en-1-yl)-3-isopropylimidazo[1,5-a]pyrazin-1-yl)-2-fluorophenyl)-1-(2-chlorophenyl)methanesulfonamide;
N-(5-(8-amino-3-isopropyl-5-(4-(oxetan-3-ylamino)cyclohex-1-en-1-yl)imidazo[1,5-a]pyrazin-1-yl)-3-fluoropyridin-2-yl)-2-fluorobenzenesulfonamide;
N-(4-(8-amino-3-isopropyl-5-(4-(oxetan-3-ylamino)cyclohex-1-en-1-yl)imidazo[1,5-a]pyrazin-1-yl)-2-fluorophenyl)-5-chloropyridine-2-sulfonamide;
N-(4-(8-amino-3-isopropyl-5-(4-(oxetan-3-ylamino)cyclohex-1-en-1-yl)imidazo[1,5-a]pyrazin-1-yl)-2-fluorophenyl)-2-methyloxazole-5-sulfonamide;
or a salt, solvate, enantiomer, diastereoisomer, isotopologue or tautomer thereof.

11. A pharmaceutical composition comprising at least one compound of claim 1 and at least one pharmaceutically acceptable carrier.

12. A method of treating a IRE1α-related disease in a subject, the method comprising administering to the subject a therapeutically effective amount of a compound, or a pharmaceutically acceptable salt, solvate, enantiomer, diastereoisomer, or tautomer thereof, of claim 1.

13. The method of claim 12, wherein the disease is selected from the group consisting of a neurodegenerative disease, a demyelinating disease, cancer, an eye disease, a fibrotic disease, and diabetes.

14. The method of claim 13, wherein at least one of the following applies:
(a) the neurodegenerative disease is selected from the group consisting of retinitis pigmentosa, amyotrophic lateral sclerosis, retinal degeneration, macular degeneration, Parkinson's Disease, Alzheimer's Disease, Huntington's Disease, Prion Disease, Creutzfeldt-Jakob Disease, and Kuru;
(b) the demyelinating disease is selected from the group consisting of Wolfram Syndrome, Pelizaeus-Merzbacher Disease, Transverse Myelitis, Charcot-Marie-Tooth Disease, and Multiple Sclerosis;
(c) the cancer is multiple myeloma;
(d) the diabetes is selected from the group consisting of type I diabetes and type II diabetes;
(e) the eye disease is selected from the group consisting of retinitis pigmentosa, retinal degeneration, macular degeneration, and Wolfram Syndrome; and
(f) the fibrotic disease is selected from the group consisting of idiopathic pulmonary fibrosis (IPF), myocardial infarction, cardiac hypertrophy, heart failure, cirrhosis, acetominophen liver toxicity, hepatitis C liver disease, hepatosteatosis (fatty liver disease), or hepatic fibrosis.

15-19. (canceled)

20. A method of inhibiting the activity of an IRE1 protein, the method comprising contacting the IRE1 protein with an effective amount of a compound, or a pharmaceutically acceptable salt thereof, of claim 1.

21. The method of claim 20, wherein the activity is selected from the group consisting of kinase activity, oligomerization activity, and RNase activity.

22. The method of claim 20, wherein one of the following applies:
(a) the IRE1 protein is within a cell;
(b) the IRE1 protein is within a cell and apoptosis of the cell is prevented or minimized;
(c) the IRE1 protein is within a cell and the cell is in an organism that has an IRE1α-related disease or disorder.

23-24. (canceled)

25. The method of claim 24, wherein the disease or disorder is a neurodegenerative disease, demyelinating disease, cancer, eye disease, fibrotic disease, or diabetes.

26. The method of claim 12, wherein the subject is need of the treatment.
